SEQUENCE LISTING (96 sequences)

SEQ ID NO: 1 - RNA, 19 nt, Artificial Sequence
  siRNA strand - modified_nucleotides, modified as per summary sequence table at the end of the description
aaccagaagaagcagguga

SEQ ID NO: 2 - RNA, 19 nt, Artificial Sequence
  siRNA strand - modified_nucleotides, modified as per summary sequence table at the end of the description
ucaccugcuucuucugguu

SEQ ID NO: 3 - DNA, 22 bp, Artificial Sequence
  primer
tggacaccaaatcgtactggaa

SEQ ID NO: 4 - DNA, 22 bp, Artificial Sequence
  primer
tggacaccaaatcgtactggaa

SEQ ID NO: 5 - DNA, 27 bp, Artificial Sequence
  probe - modified as per summary sequence table at the end of the description
acttggcatttccccgttccatgaatt

SEQ ID NO: 6 - DNA, 22 bp, Artificial Sequence
  primer
caccgccaaatttaactgcaga

SEQ ID NO: 7 - DNA, 24 bp, Artificial Sequence
  primer
aagggtttgataagttctagctgt

SEQ ID NO: 8 - DNA, 31 bp, Artificial Sequence
  probe - modified as per summary sequence table at the end of the description
tgcacagtatccttttgaagaccataaccca

SEQ ID NO: 9 - DNA, 22 bp, Artificial Sequence
  primer
ggcaagccttatgtcatctcgt

SEQ ID NO: 10 - DNA, 23 bp, Artificial Sequence
  primer
ggaatggttttcccatggtactt

SEQ ID NO: 11 - DNA, 28 bp, Artificial Sequence
  probe - modified as per summary sequence table at the end of the description
tgaaatgtctccgctattacgctggctg

SEQ ID NO: 12 - DNA, 21 bp, Artificial Sequence
  primer
cggcacctaccttccactctt

SEQ ID NO: 13 - DNA, 17 bp, Artificial Sequence
  primer
tcggtggtgggcatcct

SEQ ID NO: 14 - DNA, 28 bp, Artificial Sequence
  probe - modified as per summary sequence table at the end of the description
ccgagatgtttccagctcccctgttcta

SEQ ID NO: 15 - DNA, 18 bp, Artificial Sequence
  primer
gtgtcctcgcaacgtcca

SEQ ID NO: 16 - DNA, 15 bp, Artificial Sequence
  primer
gaccccggggctttg

SEQ ID NO: 17 - DNA, 27 bp, Artificial Sequence
  probe - modified as per summary sequence table at the end of the description
tggctgtttctgaacaagcaccaatgg

SEQ ID NO: 18 - DNA, 23 bp, Artificial Sequence
  primer
gcatgggtcagaaggattcctat

SEQ ID NO: 19 - DNA, 23 bp, Artificial Sequence
  primer
tgtagaaggtgtggtgccagatt

SEQ ID NO: 20 - DNA, 22 bp, Artificial Sequence
  probe - modified as per summary sequence table at the end of the description
tcgagcacggcatcgtcaccaa

SEQ ID NO: 21 - RNA, 21 nt, Artificial Sequence
  siRNA strand - modified_nucleotides, modified as per summary sequence table at the end of the description
uuauagagcaagaacacuguu

SEQ ID NO: 22 - RNA, 21 nt, Artificial Sequence
  siRNA strand - modified_nucleotides, modified as per summary sequence table at the end of the description
aacaguguucuugcucuauaa

SEQ ID NO: 23 - RNA, 21 nt, Artificial Sequence
  siRNA strand - modified_nucleotides, modified as per summary sequence table at the end of the description
uuauagagcaagaacacuguu

SEQ ID NO: 24 - RNA, 21 nt, Artificial Sequence
  siRNA strand - modified_nucleotides, modified as per summary sequence table at the end of the description
aacaguguucuugcucuauaa

SEQ ID NO: 25 - RNA, 21 nt, Artificial Sequence
  siRNA strand - modified_nucleotides, modified as per summary sequence table at the end of the description
uuauagagcaagaacacuguu

SEQ ID NO: 26 - RNA, 21 nt, Artificial Sequence
  siRNA strand - modified_nucleotides, modified as per summary sequence table at the end of the description
aacaguguucuugcucuauaa

SEQ ID NO: 27 - RNA, 21 nt, Artificial Sequence
  siRNA strand - modified_nucleotides, modified as per summary sequence table at the end of the description
uuauagagcaagaacacuguu

SEQ ID NO: 28 - RNA, 21 nt, Artificial Sequence
  siRNA strand - modified_nucleotides, modified as per summary sequence table at the end of the description
aacaguguucuugcucuauaa

SEQ ID NO: 29 - RNA, 21 nt, Artificial Sequence
  siRNA strand - modified_nucleotides, modified as per summary sequence table at the end of the description
uuauagagcaagaacacuguu

SEQ ID NO: 30 - RNA, 21 nt, Artificial Sequence
  siRNA strand - modified_nucleotides, modified as per summary sequence table at the end of the description
aacaguguucuugcucuauaa

SEQ ID NO: 31 - RNA, 21 nt, Artificial Sequence
  siRNA strand - modified_nucleotides, modified as per summary sequence table at the end of the description
uuauagagcaagaacacuguu

SEQ ID NO: 32 - RNA, 21 nt, Artificial Sequence
  siRNA strand - modified_nucleotides, modified as per summary sequence table at the end of the description
aacaguguucuugcucuauaa

SEQ ID NO: 33 - RNA, 21 nt, Artificial Sequence
  siRNA strand - modified_nucleotides, modified as per summary sequence table at the end of the description
uuauagagcaagaacacuguu

SEQ ID NO: 34 - RNA, 21 nt, Artificial Sequence
  siRNA strand - modified_nucleotides, modified as per summary sequence table at the end of the description
aacaguguucuugcucuauaa

SEQ ID NO: 35 - RNA, 21 nt, Artificial Sequence
  siRNA strand - modified_nucleotides, modified as per summary sequence table at the end of the description
uuauagagcaagaacacuguu

SEQ ID NO: 36 - RNA, 21 nt, Artificial Sequence
  siRNA strand - modified_nucleotides, modified as per summary sequence table at the end of the description
aacaguguucuugcucuauaa

SEQ ID NO: 37 - RNA, 21 nt, Artificial Sequence
  siRNA strand - modified_nucleotides, modified as per summary sequence table at the end of the description
uuauagagcaagaacacuguu

SEQ ID NO: 38 - RNA, 21 nt, Artificial Sequence
  siRNA strand - modified_nucleotides, modified as per summary sequence table at the end of the description
aacaguguucuugcucuauaa

SEQ ID NO: 39 - RNA, 21 nt, Artificial Sequence
  siRNA strand - modified_nucleotides, modified as per summary sequence table at the end of the description
uuauagagcaagaacacuguu

SEQ ID NO: 40 - RNA, 21 nt, Artificial Sequence
  siRNA strand - modified_nucleotides, modified as per summary sequence table at the end of the description
aacaguguucuugcucuauaa

SEQ ID NO: 41 - RNA, 21 nt, Artificial Sequence
  siRNA strand - modified_nucleotides, modified as per summary sequence table at the end of the description
uuauagagcaagaacacuguu

SEQ ID NO: 42 - RNA, 21 nt, Artificial Sequence
  siRNA strand - modified_nucleotides, modified as per summary sequence table at the end of the description
aacaguguucuugcucuauaa

SEQ ID NO: 43 - RNA, 21 nt, Artificial Sequence
  siRNA strand - modified_nucleotides, modified as per summary sequence table at the end of the description
uuauagagcaagaacacuguu

SEQ ID NO: 44 - RNA, 21 nt, Artificial Sequence
  siRNA strand - modified_nucleotides, modified as per summary sequence table at the end of the description
aacaguguucuugcucuauaa

SEQ ID NO: 45 - RNA, 19 nt, Artificial Sequence
  siRNA strand - modified_nucleotides, modified as per summary sequence table at the end of the description
ucuucuuaaacugaguuuc

SEQ ID NO: 46 - RNA, 19 nt, Artificial Sequence
  siRNA strand - modified_nucleotides, modified as per summary sequence table at the end of the description
gaaacucaguuuaagaaga

SEQ ID NO: 47 - RNA, 19 nt, Artificial Sequence
  siRNA strand - modified_nucleotides, modified as per summary sequence table at the end of the description
auguagccgaggaucuucu

SEQ ID NO: 48 - RNA, 19 nt, Artificial Sequence
  siRNA strand - modified_nucleotides, modified as per summary sequence table at the end of the description
agaagauccucggcuacau

SEQ ID NO: 49 - RNA, 19 nt, Artificial Sequence
  siRNA strand - modified_nucleotides, modified as per summary sequence table at the end of the description
aaccagaagaagcagguga

SEQ ID NO: 50 - RNA, 19 nt, Artificial Sequence
  siRNA strand - modified_nucleotides, modified as per summary sequence table at the end of the description
ucaccugcuucuucugguu

SEQ ID NO: 51 - RNA, 19 nt, Artificial Sequence
  siRNA strand - modified_nucleotides, modified as per summary sequence table at the end of the description
auaacucuguccauuaccg

SEQ ID NO: 52 - RNA, 19 nt, Artificial Sequence
  siRNA strand - modified_nucleotides, modified as per summary sequence table at the end of the description
cgguaauggacagaguuau

SEQ ID NO: 53 - RNA, 21 nt, Artificial Sequence
  siRNA strand - modified_nucleotides, modified as per summary sequence table at the end of the description
uuauagagcaagaacacuguu

SEQ ID NO: 54 - RNA, 21 nt, Artificial Sequence
  siRNA strand - modified_nucleotides, modified as per summary sequence table at the end of the description
aacaguguucuugcucuauaa

SEQ ID NO: 55 - RNA, 21 nt, Artificial Sequence
  siRNA strand - modified_nucleotides, modified as per summary sequence table at the end of the description
uuauagagcaagaacacuguu

SEQ ID NO: 56 - RNA, 21 nt, Artificial Sequence
  siRNA strand - modified_nucleotides, modified as per summary sequence table at the end of the description
aacaguguucuugcucuauaa

SEQ ID NO: 57 - RNA, 21 nt, Artificial Sequence
  siRNA strand - modified_nucleotides, modified as per summary sequence table at the end of the description
uuauagagcaagaacacuguu

SEQ ID NO: 58 - RNA, 21 nt, Artificial Sequence
  siRNA strand - modified_nucleotides, modified as per summary sequence table at the end of the description
aacaguguucuugcucuauaa

SEQ ID NO: 59 - RNA, 19 nt, Artificial Sequence
  siRNA strand - modified_nucleotides, modified as per summary sequence table at the end of the description
ucuucuuaaacugaguuuc

SEQ ID NO: 60 - RNA, 19 nt, Artificial Sequence
  siRNA strand - modified_nucleotides, modified as per summary sequence table at the end of the description
gaaacucaguuuaagaaga

SEQ ID NO: 61 - RNA, 19 nt, Artificial Sequence
  siRNA strand - modified_nucleotides, modified as per summary sequence table at the end of the description
aaccagaagaagcagguga

SEQ ID NO: 62 - RNA, 19 nt, Artificial Sequence
  siRNA strand - modified_nucleotides, modified as per summary sequence table at the end of the description
ucaccugcuucuucugguu

SEQ ID NO: 63 - RNA, 21 nt, Artificial Sequence
  siRNA strand - modified_nucleotides, modified as per summary sequence table at the end of the description
uuauagagcaagaacacuguu

SEQ ID NO: 64 - RNA, 21 nt, Artificial Sequence
  siRNA strand - modified_nucleotides, modified as per summary sequence table at the end of the description
aacaguguucuugcucuauaa

SEQ ID NO: 65 - RNA, 21 nt, Artificial Sequence
  siRNA strand - modified_nucleotides, modified as per summary sequence table at the end of the description
uuauagagcaagaacacuguu

SEQ ID NO: 66 - RNA, 21 nt, Artificial Sequence
  siRNA strand - modified_nucleotides, modified as per summary sequence table at the end of the description
aacaguguucuugcucuauaa

SEQ ID NO: 67 - RNA, 19 nt, Artificial Sequence
  siRNA strand - modified_nucleotides, modified as per summary sequence table at the end of the description
ucgaaguauuccgcguacg

SEQ ID NO: 68 - RNA, 19 nt, Artificial Sequence
  siRNA strand - modified_nucleotides, modified as per summary sequence table at the end of the description
cguacgcggaauacuucga

SEQ ID NO: 69 - RNA, 21 nt, Artificial Sequence
  siRNA strand - modified_nucleotides, modified as per summary sequence table at the end of the description
uuauagagcaagaacacuguu

SEQ ID NO: 70 - RNA, 21 nt, Artificial Sequence
  siRNA strand - modified_nucleotides, modified as per summary sequence table at the end of the description
aacaguguucuugcucuauaa

SEQ ID NO: 71 - RNA, 19 nt, Artificial Sequence
  siRNA strand - modified_nucleotides, modified as per summary sequence table at the end of the description
cuuacucucgcccaagcga

SEQ ID NO: 72 - RNA, 19 nt, Artificial Sequence
  siRNA strand - modified_nucleotides, modified as per summary sequence table at the end of the description
ucgcuugggcgagaguaag

SEQ ID NO: 73 - RNA, 19 nt, Artificial Sequence
  siRNA strand - modified_nucleotides, modified as per summary sequence table at the end of the description
ucuucuuaaacugaguuuc

SEQ ID NO: 74 - RNA, 19 nt, Artificial Sequence
  siRNA strand - modified_nucleotides, modified as per summary sequence table at the end of the description
gaaacucaguuuaagaaga

SEQ ID NO: 75 - RNA, 19 nt, Artificial Sequence
  siRNA strand - modified_nucleotides, modified as per summary sequence table at the end of the description
auguagccgaggaucuucu

SEQ ID NO: 76 - RNA, 19 nt, Artificial Sequence
  siRNA strand - modified_nucleotides, modified as per summary sequence table at the end of the description
agaagauccucggcuacau

SEQ ID NO: 77 - RNA, 19 nt, Artificial Sequence
  siRNA strand - modified_nucleotides, modified as per summary sequence table at the end of the description
uaccagaagaagcagguga

SEQ ID NO: 78 - RNA, 19 nt, Artificial Sequence
  siRNA strand - modified_nucleotides, modified as per summary sequence table at the end of the description
ucaccugcuucuucuggua

SEQ ID NO: 79 - RNA, 19 nt, Artificial Sequence
  siRNA strand - modified_nucleotides, modified as per summary sequence table at the end of the description
aaccagaagaagcagguga

SEQ ID NO: 80 - RNA, 19 nt, Artificial Sequence
  siRNA strand - modified_nucleotides, modified as per summary sequence table at the end of the description
ucaccugcuucuucugguu

SEQ ID NO: 81 - RNA, 19 nt, Artificial Sequence
  siRNA strand - modified_nucleotides, modified as per summary sequence table at the end of the description
ucgaaguauuccgcguacg

SEQ ID NO: 82 - RNA, 19 nt, Artificial Sequence
  siRNA strand - modified_nucleotides, modified as per summary sequence table at the end of the description
cguacgcggaauacuucga

SEQ ID NO: 83 - RNA, 19 nt, Artificial Sequence
  siRNA strand - modified_nucleotides, modified as per summary sequence table at the end of the description
uuauagagcaagaacacug

SEQ ID NO: 84 - RNA, 19 nt, Artificial Sequence
  siRNA strand - modified_nucleotides, modified as per summary sequence table at the end of the description
caguguucuugcucuauaa

SEQ ID NO: 85 - RNA, 19 nt, Artificial Sequence
  siRNA strand - modified_nucleotides, modified as per summary sequence table at the end of the description
uuauagagcaagaacacug

SEQ ID NO: 86 - RNA, 19 nt, Artificial Sequence
  siRNA strand - modified_nucleotides, modified as per summary sequence table at the end of the description
caguguucuugcucuauaa

SEQ ID NO: 87 - RNA, 19 nt, Artificial Sequence
  siRNA strand - modified_nucleotides, modified as per summary sequence table at the end of the description
uuauagagcaagaacacug

SEQ ID NO: 88 - RNA, 19 nt, Artificial Sequence
  siRNA strand - modified_nucleotides, modified as per summary sequence table at the end of the description
caguguucuugcucuauaa

SEQ ID NO: 89 - RNA, 19 nt, Artificial Sequence
  siRNA strand - modified_nucleotides, modified as per summary sequence table at the end of the description
aaccagaagaagcagguga

SEQ ID NO: 90 - RNA, 19 nt, Artificial Sequence
  siRNA strand - modified_nucleotides, modified as per summary sequence table at the end of the description
ucaccugcuucuucugguu

SEQ ID NO: 91 - RNA, 19 nt, Artificial Sequence
  siRNA strand - modified_nucleotides, modified as per summary sequence table at the end of the description
aaccagaagaagcagguga

SEQ ID NO: 92 - RNA, 19 nt, Artificial Sequence
  siRNA strand - modified_nucleotides, modified as per summary sequence table at the end of the description
ucaccugcuucuucugguu

SEQ ID NO: 93 - RNA, 19 nt, Artificial Sequence
  siRNA strand - modified_nucleotides, modified as per summary sequence table at the end of the description
ucuucuuaaacugaguuuc

SEQ ID NO: 94 - RNA, 19 nt, Artificial Sequence
  siRNA strand - modified_nucleotides, modified as per summary sequence table at the end of the description
gaaacucaguuuaagaaga

SEQ ID NO: 95 - RNA, 19 nt, Artificial Sequence
  siRNA strand - modified_nucleotides, modified as per summary sequence table at the end of the description
ucuucuuaaacugaguuuc

SEQ ID NO: 96 - RNA, 19 nt, Artificial Sequence
  siRNA strand - modified_nucleotides, modified as per summary sequence table at the end of the description
gaaacucaguuuaagaaga

(19) United States
(12) Patent Application Publication
(10) Pub. No.: US 2021/0155926 A1
(43) Pub. Date: May 27, 2021

(54) siRNAs WITH AT LEAST TWO LIGANDS AT DIFFERENT ENDS

(71) Applicant: SILENCE THERAPEUTICS GMBH, Berlin (DE)

(72) Inventors: Lucas Bethge, Potsdam (DE); Adrien Weingärtner, Berlin (DE); Judith Hauptmann, Berlin (DE)

(21) Appl. No.: 17/045,125

(22) PCT Filed: Apr. 5, 2019
(86) PCT No.: PCT/EP2019/058721

§ 371 Date: Oct. 2, 2020

(30) Foreign Application Priority Data
Apr. 5, 2018 (EP) 18165917.8
Apr. 5, 2018 (EP) 18165986.3
Jul. 5, 2018 (GB) 1811074.2
Jul. 5, 2018 (GB) 1811084.1
Sep. 28, 2018 (GB) 1815836.0
Sep. 28, 2018 (GB) 1815925.1

(51) Int. Cl. C12N 15/113; A61K 31/7125

(52) U.S. Cl. CPC C12N 15/113; A61K 31/7125; C12N 2310/3519; C12N 2310/315; C12N 2310/322; C12N 2310/321

(57) ABSTRACT

There is provided inter alia a conjugate for inhibiting expression of a target gene in a cell, said conjugate comprising a nucleic acid portion and ligand portions, said nucleic acid portion comprising at least one duplex region that comprises at least a portion of a first RNA strand and at least a portion of a second RNA strand that is at least partially complementary to the first strand, wherein said first strand is at least partially complementary to at least a portion of RNA transcribed from said target gene, said ligand portions comprising a linker moiety and a targeting ligand for in vivo targeting of cells and being conjugated exclusively to the 3′ and/or 5′ ends of one or both RNA strands, wherein the 5′ end of the first RNA strand is not conjugated, wherein: (i) the second RNA strand is conjugated at the 5′ end to the targeting ligand, and wherein (a) the second RNA strand is also conjugated at the 3′ end to the targeting ligand and the 3′ end of the first RNA strand is not conjugated; or (b) the first RNA strand is conjugated at the 3′ end to the targeting ligand and the 3′ end of the second RNA strand is not conjugated; or (c) both the second RNA strand and the first RNA strand are also conjugated at the 3′ ends to the targeting ligand; or (ii) both the second RNA strand and the first RNA strand are conjugated at the 3′ ends to the targeting ligand and the 5′ end of the second RNA strand is not conjugated.

### TECHNICAL FIELD

The present invention relates to novel nucleic acid conjugate compounds. The invention further relates to compositions comprising said conjugates and their use in medicine, research and diagnostics. The novel conjugate compounds may be used in the treatment of many diseases including central-nervous-system diseases, inflammatory diseases, metabolic disorders, genetic and inherited diseases, oncology, infectious diseases, and ocular disease.

### BACKGROUND

Double-stranded RNA (dsRNA) has been shown to block gene expression (Fire et al., 1998 and Elbashir et al., 2001) and this has been termed RNA interference or “RNAi”, mediated by interfering RNA molecules (RNAi). Short dsRNA directs gene-specific, post-transcriptional silencing in many organisms, including vertebrates, and has provided a new tool for studying gene function. RNAi is mediated by RNA-induced silencing complex (RISC), a sequence-specific, multi-component nuclease that destroys messenger RNAs homologous to the silencing trigger. iRNAs (interfering RNA) such as siRNA (short interfering RNA), antisense RNA, and micro-RNA are oligonucleotides that prevent the formation of proteins by gene-silencing i.e. inhibiting translation of the protein. Gene-silencing agents are becoming increasingly important for therapeutic applications in medicine. Thus, means for efficient delivery of oligonucleotides, in particular double stranded siRNAs, to cells in vivo are becoming increasingly important and require specific targeting and substantial protection from the extracellular environment, particularly serum proteins. One method of achieving specific targeting is to conjugate a targeting moiety to the RNAi duplex agent. The targeting moiety helps in targeting the RNAi duplex agent to the required target site and there is a need to design appropriate targeting moieties for the desired receptor sites for the conjugated molecules to be taken up by the cells such as by endocytosis. For example, the asialoglycoprotein receptor (ASGPR) is a high capacity receptor, which is highly abundant on hepatocytes and it has been known for more than ten years that GalNAc-siRNA conjugates are sufficient to target and deliver siRNA into hepatocytes in vivo. While in the past, trimeric clusters were preferred due to higher binding affinity to the ASGPR, some more recent publications report on two single GalNAc moieties to be sufficient.

Matsuda et al. (2015) describes conjugates which have conjugation of the RNA strands to GalNAc within the RNA strands. Schmidt et al. (2017) describes one, two or three-GalNAc conjugated single stranded and double stranded DNA antisense oligonucleotides (ASOs). Kamiya et al. (2014) discloses a study which investigates terminal substitution of siRNAs. However, targeting ligands developed so far do not always translate to in vivo setting and there is a clear need for more efficacious receptor specific ligand conjugated RNAi duplex agents and methods for their preparation for the in vivo delivery of oligonucleotide therapeutics, nucleic acids and double stranded siRNAs. The present invention attempts to address these needs.

The present invention relates to the finding that nucleic acid conjugates of particular structures are potent with long duration of action and have surprising improved in vivo activity over other nucleic acid conjugates.

### SUMMARY OF THE INVENTION

The present invention relates to a conjugate for inhibiting expression of a target gene in a cell, said conjugate comprising a nucleic acid portion and ligand portions, said nucleic acid portion comprising at least one duplex region that comprises at least a portion of a first RNA strand and at least a portion of a second RNA strand that is at least partially complementary to the first strand, wherein said first strand is at least partially complementary to at least a portion of RNA transcribed from said target gene, said ligand portions comprising a linker moiety and a targeting ligand for in vivo targeting of cells and being conjugated exclusively to the 3′ and/or 5′ ends of one or both RNA strands, wherein the 5′ end of the first RNA strand is not conjugated, wherein:
(i) the second RNA strand is conjugated at the 5′ end to the targeting ligand, and wherein (a) the second RNA strand is also conjugated at the 3′ end to the targeting ligand and the 3′ end of the first RNA strand is not conjugated; or (b) the first RNA strand is conjugated at the 3′ end to the targeting ligand and the 3′ end of the second RNA strand is not conjugated; or (c) both the second RNA strand and the first RNA strand are also conjugated at the 3′ ends to the targeting ligand; or
(ii) both the second RNA strand and the first RNA strand are conjugated at the 3′ ends to the targeting ligand and the 5′ end of the second RNA strand is not conjugated.

The linker moiety may for example be a serinol-derived linker moiety or one of the other linker types described herein.

As used herein, the first strand may be referred to as the antisense strand or the A strand and the second strand may be referred to as the sense strand or the B strand. The terms first strand and antisense strand or second strand and sense strand should be treated as interchangeable.

In the various aspects of the invention (unless the stated otherwise) the targeting ligand may be any targeting ligand appropriate for the cell to be targeted. In one preferred embodiment, the targeting ligand targets ASGP receptors, especially such receptors on liver cells. For example, the targeting ligand is or comprises a saccharide moiety such as galactose, mannose, glucose, glucosamine, fucose and fructose or derivatives thereof such as N-acetyl derivatives thereof e.g. GalNAc. The preferred targeting ligand is GalNAc.

### BRIEF DESCRIPTION OF FIGURES

FIG. 1—depicts Conjugate 1. The last three nucleotides at the 5′ and 3′ ends of the antisense and sense strands are connected by a phosphorothioate linkage between each nucleotide. The serinol-GalNAc-linkers are conjugated via a phosphorothioate bond to the 3′ end of the antisense strand as well as to the 5′ end of the sense strand.

FIG. 2—depicts Conjugate 2. The last three nucleotides at the 5′ and 3′ ends of the antisense and sense strands are connected by a phosphorothioate linkage between each nucleotide. The serinol-GalNAc-linkers are conjugated via a phosphorothioate bond to the 3′ end and the 5′ end of the sense strand.

FIG. 3—depicts Conjugate 3. The last three nucleotides at the 5′ and 3′ ends of the antisense and sense strands are connected by a phosphorothioate linkage between each nucleotide. The serinol-GalNAc-linkers are conjugated via a phosphorothioate bond to the 3′ end and the 5′ end of the sense strand as well as to the 3′ end of the antisense strand.

FIG. 4—depicts Conjugate 4. The last three nucleotides at the 5′ and 3′ ends of the antisense and sense strands are connected by a phosphorothioate linkage between each nucleotide. The serinol-GalNAc-linkers are conjugated via a phosphodiester bond to the 3′ end and the 5′ end of the sense strand.

FIG. 5—depicts Conjugate 5. The last three nucleotides at the 5′ and 3′ ends of the antisense strand are connected by a phosphorothioate linkage between each nucleotide. All remaining nucleotides of the sense strand are connected via phosphodiester bonds. The serinol-GalNAc-linkers are conjugated via a phosphorothioate bond to the 3′ end and the 5′ end of the sense strand.

FIG. 6—depicts Conjugate 6. The last three nucleotides at the 5′ and 3′ ends of the antisense strand are connected by a phosphorothioate linkage between each nucleotide. All remaining nucleotides of the sense strand are connected via phosphodiester bonds. The serinol-GalNAc-linkers are conjugated via a phosphodiester bond to the 3′ end and the 5′ end of the sense strand.

FIG. 7—depicts Conjugate 7. The last three nucleotides at the 5′ and 3′ ends of the antisense and sense strands are connected by a phosphorothioate linkage between each nucleotide. The two serinol-GalNAc-linker units are conjugated via a phosphorothioate bond to the 3′ end and the 5′ end of the sense strand. The serinol-GalNAc-linkers are connected to each other via a phosphorothioate linkage.

FIG. 8—depicts Conjugate 8. The last three nucleotides at the 5′ and 3′ ends of the antisense and sense strands are connected by a phosphorothioate linkage between each nucleotide. A GalNAc-C6-amino-modifier linker is conjugated at the 5′ end of the sense strand and a GalNAc-C7-amino-modifier linker is conjugated at the 3′ end of the sense strand via a phosphorothioate linkage.

FIG. 9—depicts Conjugate 9. The last three nucleotides at the 5′ and 3′ ends of the antisense and sense strands are connected by a phosphorothioate linkage between each nucleotide. A GalNAc-GlyC3-amino-modifier linker is conjugated at the 5′ and 3′ ends of the sense strand via a phosphorothioate linkage.

FIG. 10—depicts Conjugate 10. The last three nucleotides at the 5′ and 3′ ends of the antisense and sense strands are connected by a phosphorothioate linkage between each nucleotide. A GalNAc-piperidyl-amino-modifier linker is conjugated at the 5′ and 3′ ends of the sense strand via a phosphorothioate linkage.

FIG. 11—depicts Conjugate 11. The last three nucleotides at the 5′ and 3′ ends of the antisense and sense strands are connected by a phosphorothioate linkage between each nucleotide. A GalNAc-C3-amino-modifier linker is conjugated at the 5′ and 3′ ends of the sense strand via a phosphorothioate linkage.

FIG. 12—depicts Conjugate 12. The last three nucleotides at the 5′ and 3′ ends of the antisense and sense strands are connected by a phosphorothioate linkage between each nucleotide. A GalNAc-C6-amino-modifier linker is conjugated at the 5′ end of the sense strand via a phosphorothioate linkage and a GalNAc-GlyC3-amino-modifier linker is conjugated at the 3′ end of the sense strand via a phosphorothioate linkage.

FIG. 13—depicts Conjugates 15, 16, 18 and 19 which differ only by their RNA sequences. The last three nucleotides at the 5′ and 3′ ends of the antisense and sense strands are connected by a phosphorothioate linkage between each nucleotide in each conjugate. The serinol-GalNAc-linkers are conjugated via a phosphorothioate linkage to the 3′ end and the 5′ end of the sense strand.

FIG. 14—depicts Reference Conjugate 1. The last three nucleotides at the 5′ and 3′ ends of the antisense and sense strands are connected by a phosphorothioate linkage between each nucleotide. The serinol-GalNAc-linker is conjugated via a phosphorothioate bond to the 5′ end of the sense strand.

FIG. 15—depicts Reference Conjugate 2. The last three nucleotides at the 5′ and 3′ ends of the antisense and sense strands are connected by a phosphorothioate linkage between each nucleotide. The serinol-GalNAc-linker is conjugated via a phosphorothioate bond to the 3′ end of the antisense strand.

FIG. 16—depicts Reference Conjugate 3. The last three nucleotides at the 5′ and 3′ ends of the antisense and the 3′ end of the sense strands are connected by a phosphorothioate linkage between each nucleotide. The trimeric GalNAc-linker is conjugated via a phosphorothioate bond to the 5′ end of the sense strand.

FIG. 17—depicts Reference Conjugate 4. The last three nucleotides at the 5′ and 3′ ends of the antisense and the 3′ end of the sense strands are connected by a phosphorothioate linkage between each nucleotide. The trimeric GalNAc-linker is conjugated via a phosphorothioate bond to the 5′ end of the sense strand.

FIG. 18—depicts Reference Conjugate 5. The last three nucleotides at the 5′ and 3′ ends of the antisense strand and 3′ end of the sense strand are connected by a phosphorothioate linkage between each nucleotide. The trimeric GalNAc-linker is conjugated via a phosphorothioate bond to the 5′ end of the sense strand.

FIG. 19—depicts Reference Conjugate 6 and Reference Conjugate 7 which differ only by their RNA sequences. The last three nucleotides at the 5′ and 3′ ends of the antisense strand and 3′ end of the sense strand are connected by a phosphorothioate linkage between each nucleotide in both conjugates. The trimeric GalNAc-linker is conjugated via a phosphorothioate bond to the 5′ end of the sense strand in both conjugates.

FIG. 20—depicts Reference Conjugate 8. The last three nucleotides at the 5′ and 3′ ends of the antisense strand and 3′ end of the sense strand are connected by a phosphorothioate linkage between each nucleotide. The trimeric GalNAc-linker is conjugated via a phosphorothioate bond to the 5′ end of the sense strand.

In each of FIGS. 1-20, 36-38, and 41-43 the top strand is the antisense strand and the bottom strand is the sense strand i.e.

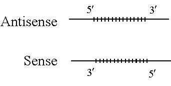

In addition, to show more clearly the connection between the nucleic acid and ligand portions, the nucleotide at the end of the respective conjugated strands is drawn in full.

FIG. 21—shows the synthesis of A0268 which is a 3′ mono-GalNAc conjugated single stranded oligonucleotide and is the starting material in the synthesis of Conjugate 1 and Conjugate 3. (ps) denotes phosphorothioate linkage.

FIG. 22—shows the synthesis of A0006 which is a 5′ tri-antennary GalNAc conjugated single stranded oligonucleotide used for the synthesis of Reference Conjugate 4. (ps) denotes phosphorothioate linkage.

FIG. 23—illustrates the in vitro determination of TTR knockdown. In particular, FIG. 23A shows the in vitro determination of TTR knockdown by Reference Conjugates (RC) 1 and 3 as well as the untreated control “UT”; FIG. 23B shows the in vitro determination of TTR knockdown by Reference Conjugates (RC) 2 and 3, as well as by the untreated control “UT”; and FIG. 23C shows the in vitro determination of TTR knockdown by Conjugates 1, 2 and 3, as well as by RC3 and untreated control “UT”. Reference Conjugates 1 and 2 represent comparator conjugates. Reference Conjugate 3 represents a non-targeting GalNAc siRNA and “untreated” (“UT”) represents untreated cells. Both RC3 and UT are negative controls. mRNA level were normalised against PtenII.

FIG. 24—illustrates the in vitro determination of TTR knockdown. In particular, FIG. 24A shows the in vitro determination of TTR knockdown by Conjugates 4, 5, 6 and 2 compared to “Luc” (Reference Conjugate 3) as well as the untreated control “UT”; FIG. 24B shows the in vitro determination of TTR knockdown by Conjugates 7 and 2, compared to “Luc” (Reference Conjugate 3) as well as the untreated control “UT”. Luc or Reference Conjugate 3 (RC3) represents a non-targeting GalNAc siRNA and “untreated” (“UT”) represents untreated cells. Both RC3 and UT are negative controls. mRNA level were normalised against Pten.

FIG. 25—illustrates the in vitro determination of TTR knockdown. In particular, FIG. 25A shows the in vitro determination of TTR knockdown by Conjugates 8, 9, 10, 11 and 2 compared to “Luc” (Reference Conjugate 3) as well as the untreated control “UT”; FIG. 25B shows the in vitro determination of TTR knockdown by Conjugates 12 and 2, compared to “Luc” (Reference Conjugate 3) as well as the untreated control “UT”. Luc or Reference Conjugate 3 represents a non-targeting GalNAc siRNA and “untreated” (“UT”) represents untreated cells. Both RC3 and UT are negative controls. mRNA level were normalised against Pten.

FIG. 26—illustrates the in vitro determination of LPA knockdown of Conjugate 19 compared to controls. Ctr represents a non-targeting GalNAc siRNA and “untreated” (“UT”) represents untreated cells. Both Ctr and UT are negative controls. mRNA level were normalised against ACTB.

FIG. 27—shows a time course of serum TTR in c57BL/6 mice cohorts of n=4 at 7, 14, and 27 days post s.c. treatment with 1 mg/kg—Conjugates 1-3, Reference Conjugates (RC) 1, 2 and 4 and mock treated (PBS) individuals.

FIG. 28—shows a time course of ALDH2 transcript level in liver of c57BL/6 mice cohorts of n=6 at 14, 28 and 42 days post s.c. treatment with 1 mg/kg—Conjugate 15, Reference Conjugate (RC) 6 and mock treated (PBS) individuals. mRNA level were normalised against Pten.

FIG. 29—shows a time course of ALDH2 transcript level in liver of c57BL/6 mice cohorts of n=6 at 14, 28 and 42 days post s.c. treatment with 1 mg/kg—Conjugate 16, Reference Conjugate (RC) 7 and mock treated (PBS) individuals. mRNA level were normalised against Pten.

FIG. 30—shows a time course of TMPRSS6 transcript level in liver of c57BL/6 mice cohorts of n=6 at 14, 28 and 42 days post s.c. treatment with 1 mg/kg—Conjugate 18, Reference Conjugate (RC) 8 and mock treated (PBS) individuals. mRNA level were normalised against Pten.

FIG. 31—shows RNA stability in tritosome lysates for indicated times at 37° C. at pH 5 of Conjugates 4, 5, 6, 7 and 2, and untreated sample (UT).

FIG. 32—shows RNA stability in tritosome lysates for indicated times at 37° C. at pH 5 of Conjugates 8, 9, 10, 11, 12 and 2, and untreated sample (UT).

FIG. 33—shows a time course of serum TTR in c57BL/6 mice cohorts of n=4 at −6, 8, 22, and 43 days post s.c. treatment on day 1 with 0.3 mg/kg—Conjugates 2, 7 or mock treated (PBS) individuals.

FIG. 34—shows RNA stability in tritosome lysates for indicated times at 37° C. at pH 5 of Conjugates 20, 21, 22 and 2, and untreated sample (UT).

FIG. 35—shows the in vitro determination of TTR knockdown of conjugates 20, 21, 22 in comparison to conjugate 2 as well as by the untreated control “ut”; and the non-targeting GalNAc siRNA (Ctr). mRNA level were normalised against Pten.

FIG. 36—depicts Conjugate 20. The last three nucleotides at the 5′ and 3′ ends of the antisense and sense strands are connected by a phosphorothioate linkage between each nucleotide. The Aspartol-GalNAc-linkers are conjugated via a phosphorothioate bond to the 5′ end of the sense strand.

FIG. 37—depicts Conjugate 21. The last three nucleotides at the 5′ and 3′ ends of the antisense and sense strands are connected by a phosphorothioate linkage between each nucleotide. The Hydroxyprolinol-GalNAc-linkers are conjugated via a phosphorothioate bond to the 5′ end of the sense strand.

FIG. 38—depicts Conjugate 22. The last three nucleotides at the 5′ and 3′ ends of the antisense and sense strands are connected by a phosphorothioate linkage between each nucleotide. The serinol-C6-GalNAc-linkers are conjugated via a phosphorothioate bond to the 5′ end of the sense strand.

FIG. 39—shows in vivo activity of GalNAc-siRNA conjugates with two different sense strand modification patterns. In both conjugates, serinol-linked GalNAc moieties are positioned at the 5′ and 3′ ends of the second strand. A conjugate with 2′-F at positions 7 8 and 9 of the second strand has increased activity as compared to one with alternating 2′-F/2′-OMe in the second strand.

FIG. 40—shows in vitro activity of GalNAc-siRNA conjugates with two different sense strand modification patterns. A conjugate with monomeric GalNAc at both termini of the second strand and with 2′-F at positions 7, 8 and 9 of the second strand has increased activity as compared to a conjugate with triantennary GalNAc at the 5′ end and alternating 2′-F/2′-OMe in the second strand.

FIG. 41—depicts Conjugate 23. The last three nucleotides at the 5′ and 3′ ends of the antisense and sense strands are connected by a phosphorothioate linkage between each nucleotide. The serinol-GalNAc-linkers are conjugated via a phosphorothioate bond to the 3′ end and the 5′ end of the sense strand. The antisense strand is alternating 2′-OMe and 2′-F starting with 2′-Ome ant the 5′-end. The sense strand is 2′-OMe modified except nucleotides 7-9 from the 5′-end which are 2′-F modified.

FIG. 42—depicts Conjugate 24. The last three nucleotides at the 5′ and 3′ ends of the antisense and sense strands are connected by a phosphorothioate linkage between each nucleotide. The serinol-GalNAc-linkers are conjugated via a phosphorothioate bond to the 3′ end and the 5′ end of the sense strand. The antisense strand is alternating 2′-OMe and 2′-F starting with 2′-OMe at the 5′-end. The sense strand is 2′-OMe modified except nucleotides 7-9 from the 5′-end which are 2′-F modified.

FIG. 43—depicts Reference Conjugate 9. The last three nucleotides at the 5′ and 3′ ends of the antisense strand and 3′ end of the sense strand are connected by a phosphorothioate linkage between each nucleotide. The trimeric GalNAc-linker is conjugated via a phosphorothioate bond to the 5′ end of the sense strand.

FIG. 44—discloses GalNac conjugates with each one serinol-linked GalNAc moiety at both termini of the second strand and with 2′-F at positions 7-9 of the second strand reducing TTR mRNA levels in vitro.

FIG. 45—discloses GalNac conjugates with each one serinol-linked GalNAc moiety at both termini of the second strand and with 2′-F at positions 7-9 of the second strand reducing TMPRSS6 mRNA levels in vitro.

FIG. 46—discloses GalNac conjugates with each one serinol-linked GalNAc moiety at both termini of the second strand and with 2′-F at positions 7-9 of the second strand reducing ALDH2 mRNA levels in vitro.

FIG. 47—shows down regulation of serum TTR levels of c57BL/6 mice at day 7 post s.c. treatment with 0.3 mg/kg of siRNA-conjugates 2, 9, 10, and 20 or with vehicle control (PBS).

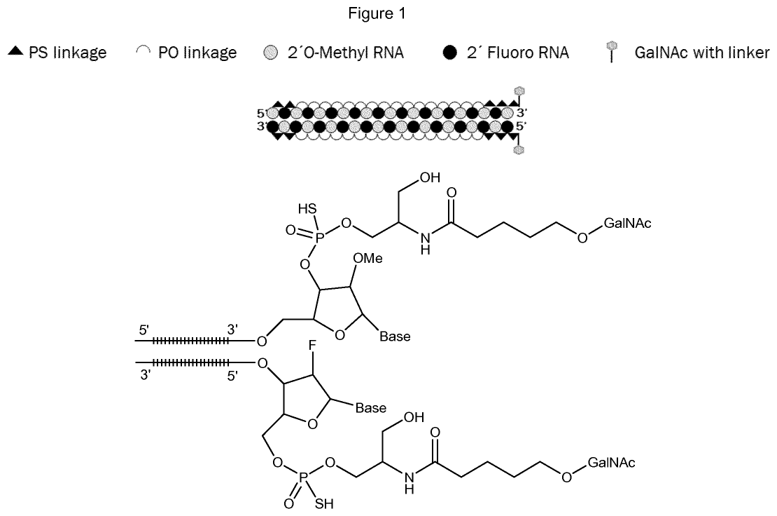

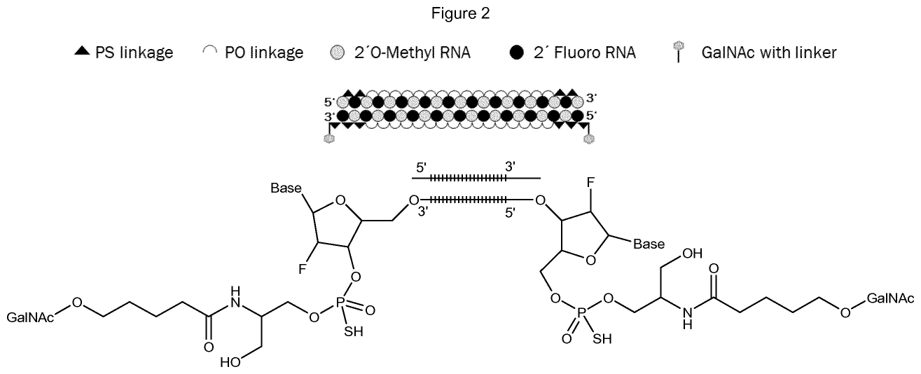

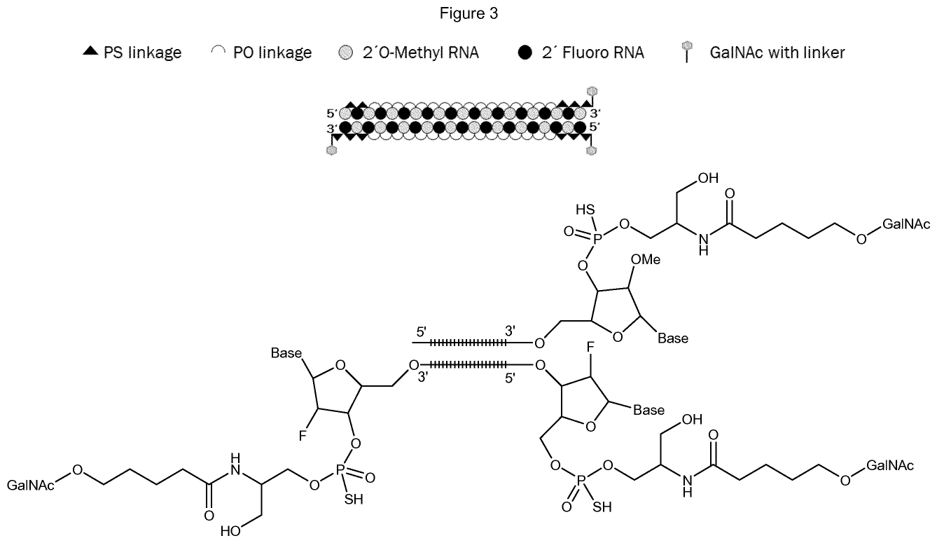

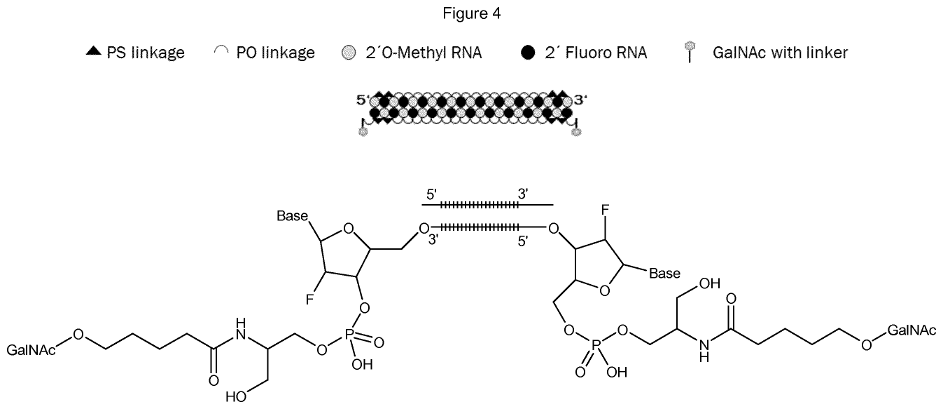

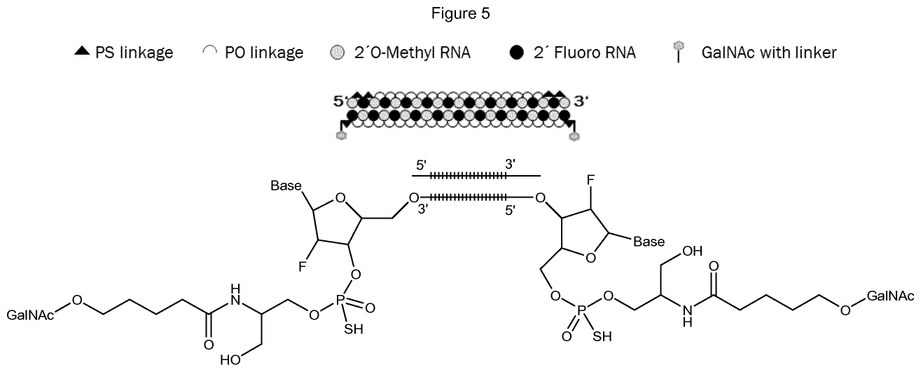

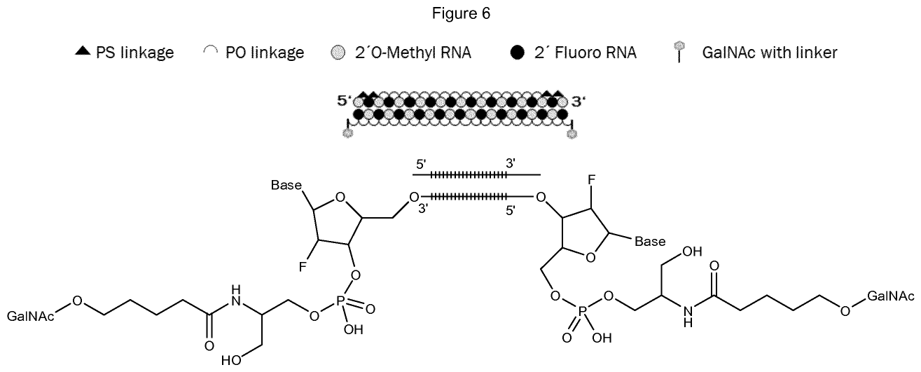

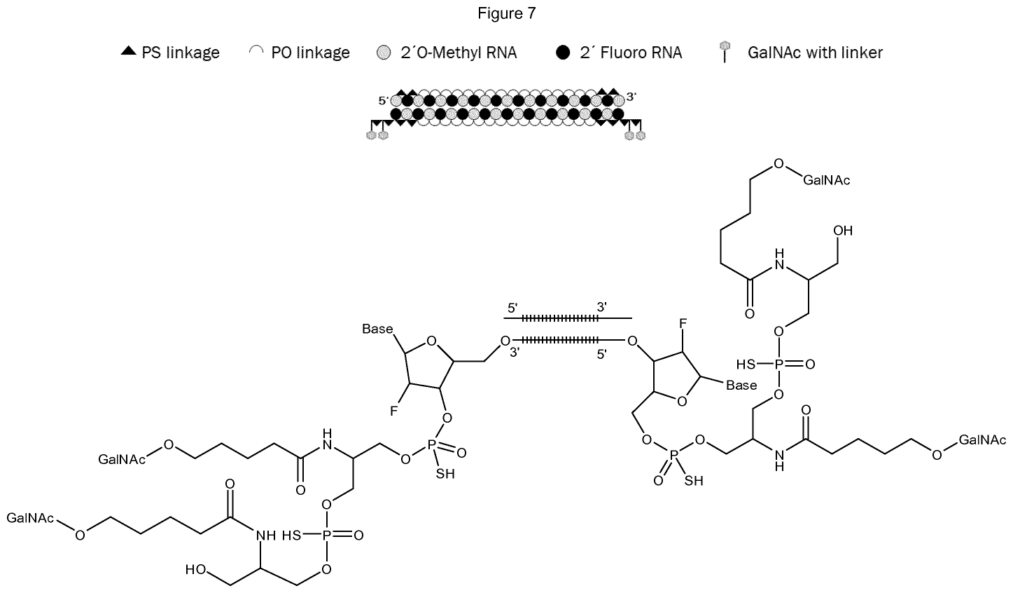

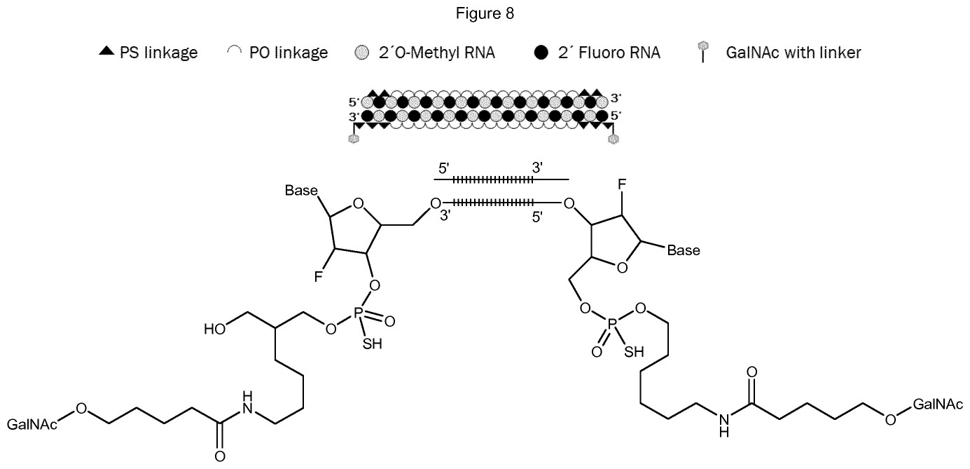

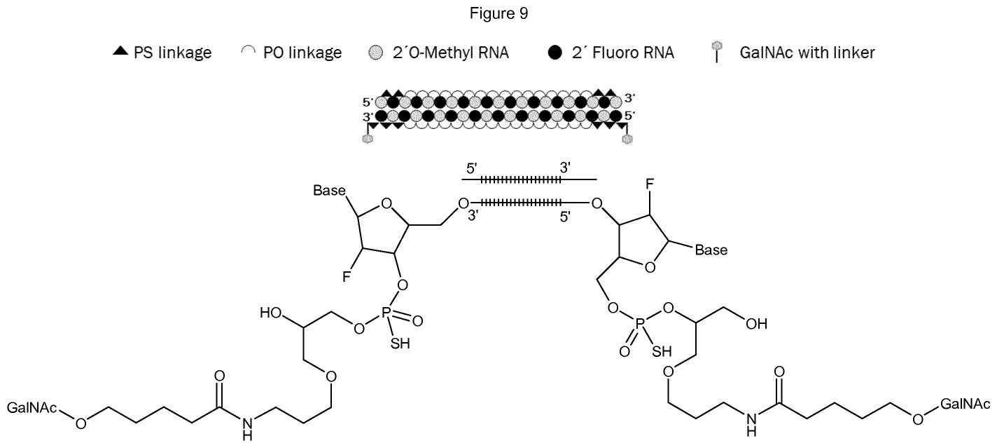

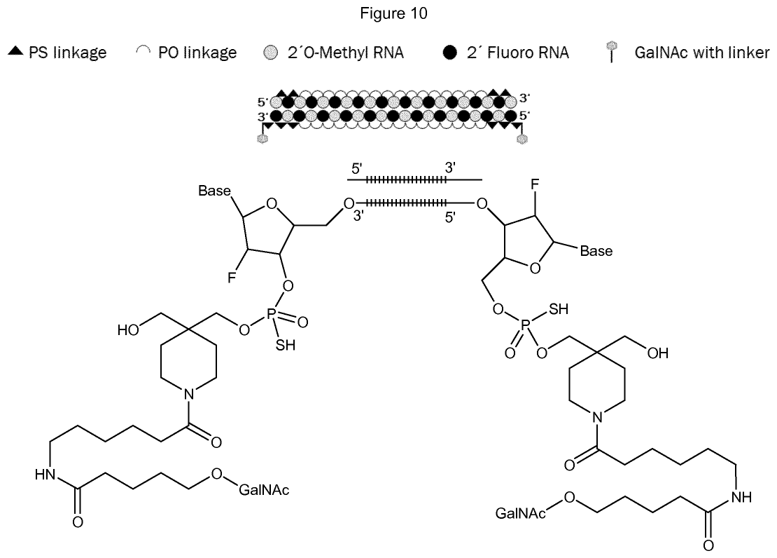

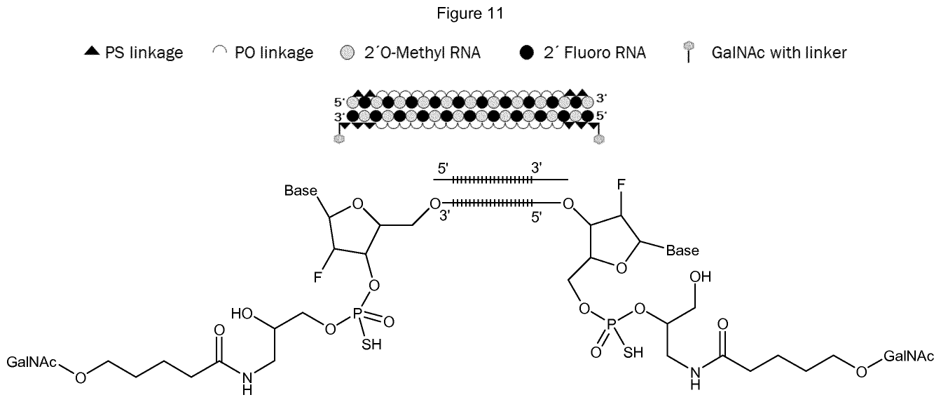

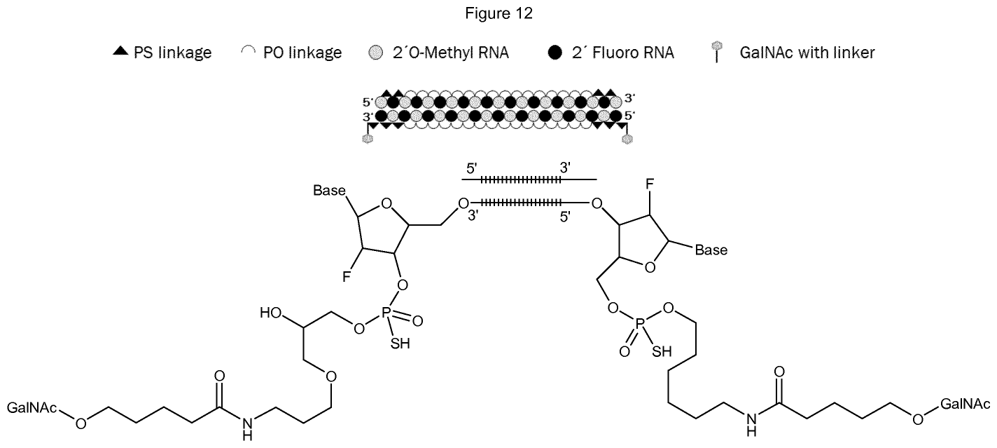

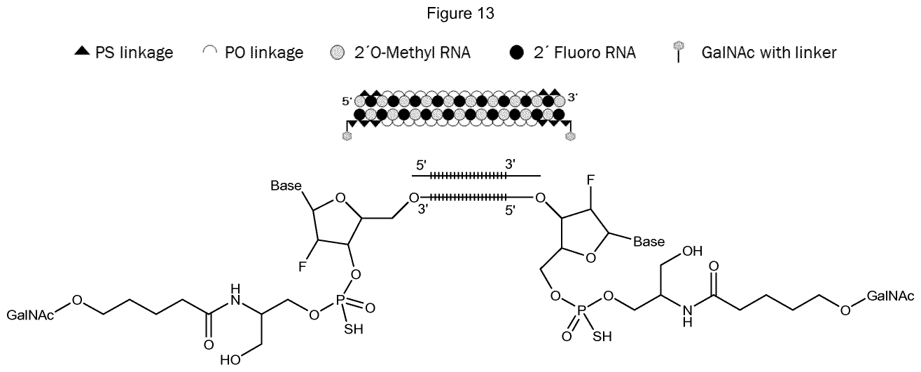

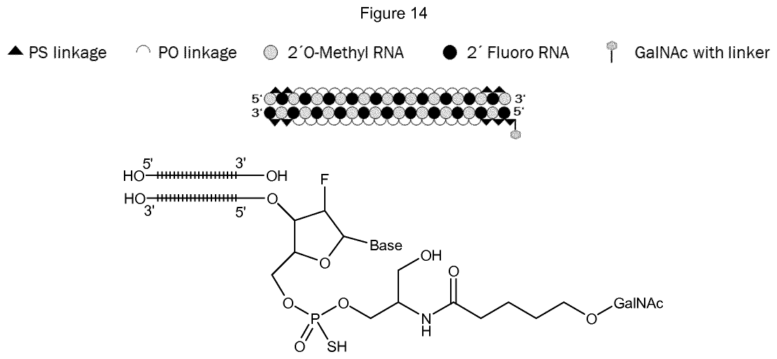

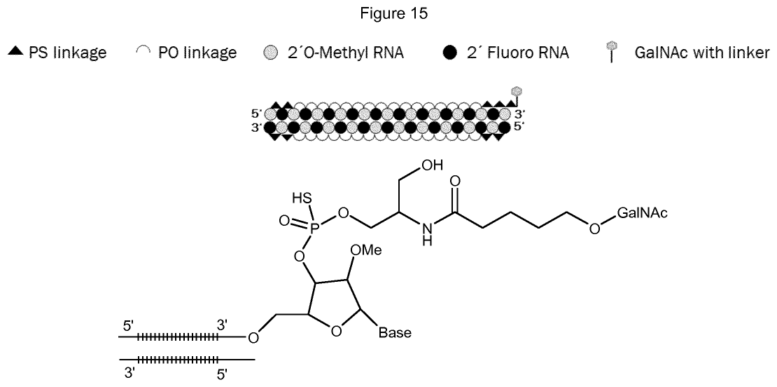

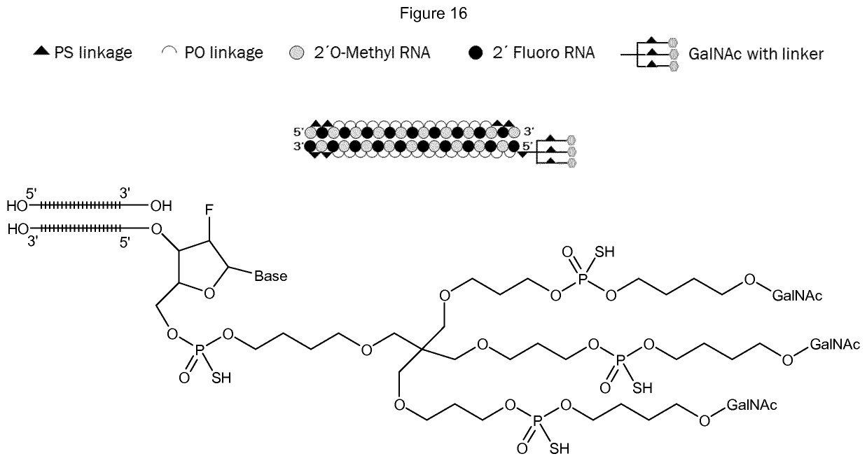

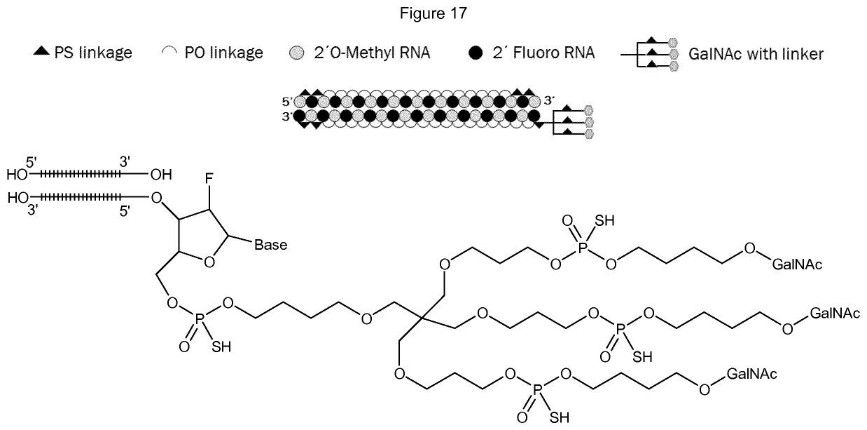

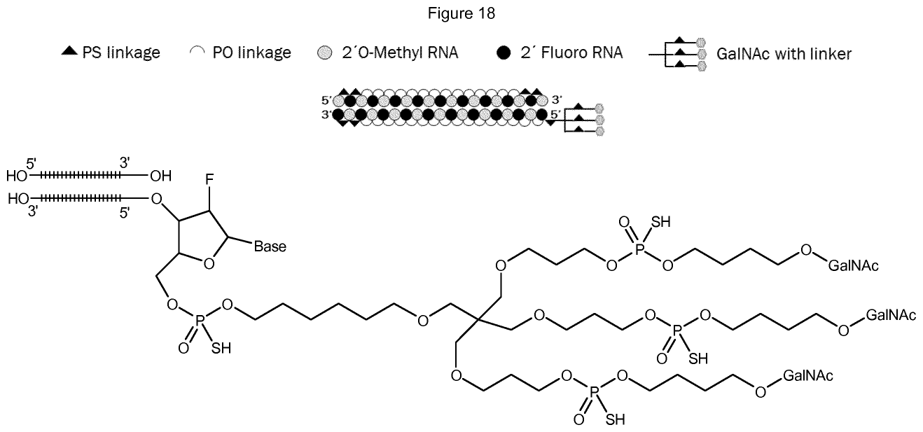

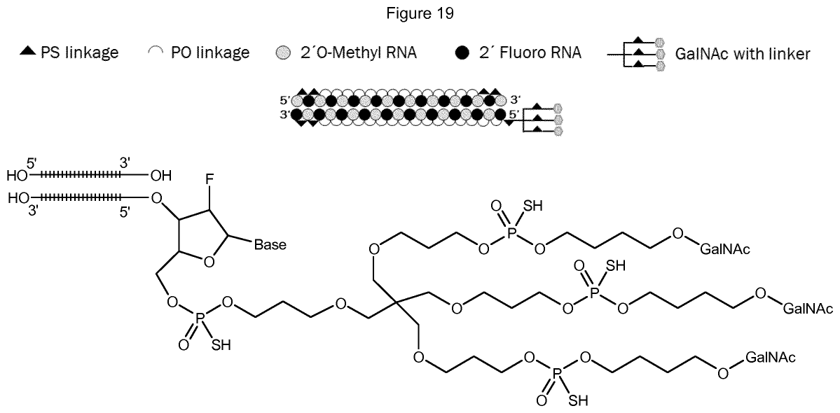

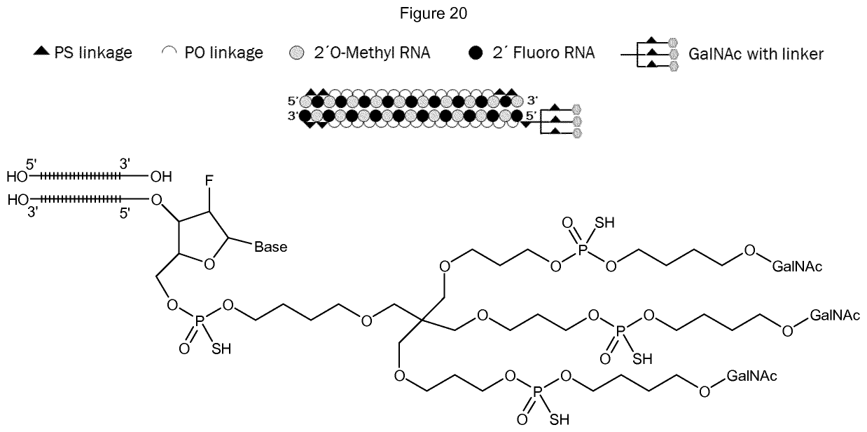

### DETAILED DESCRIPTION OF THE INVENTION

The definitions and explanations below are for the terms as used throughout this entire document including both the specification and the claims.

Unless specified otherwise, the following terms have the following meanings:

“GalNAc” means N-acetyl galactosamine which is also known as 2-(Acetylamino)-2-deoxy-D-galactopyranose. Reference to “GalNAc” or “N-acetyl galactosamine” includes both the beta-form: 2-(Acetylamino)-2-deoxy-beta-D-galactopyranose and the alpha-form: 2-(Acetylamino)-2-deoxy-alpha-D-galactopyranose. In certain embodiments, both the beta-form: 2-(Acetylamino)-2-deoxy-beta-D-galactopyranose and alpha-form: 2-(Acetylamino)-2-deoxy-alpha-D-galactopyranose may be used interchangeably. Preferably, the compounds of the invention comprise the beta-form, 2-(Acetylamino)-2-deoxy-beta-D-galactopyranose.

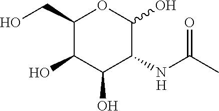

### 2-(Acetylamino)-2-deoxy-D-galactopyranose

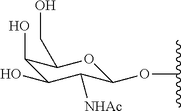 [SMILES: *OC1OC(CO)C(O)C(O)C1NC(C)=O]

### 2-(Acetylamino)-2-deoxy-beta-D-galactopyranose

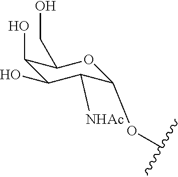 [SMILES: *OC1OC(CO)C(O)C(O)C1NC(C)=O]

### 2-(Acetylamino)-2-deoxy-alpha-D-galactopyranose

The term “C₁-C₁₅ alkyl” refers to a saturated aliphatic hydrocarbon group having 1-15 carbon atoms which may be linear or branched. For example C₁-C₆ alkyl and includes C₁ (CH₃), C₂ (CH₂CH₃), C₃ ((CH₂)₂CH₃), C₄ ((CH₂)₃CH₃), C₅ ((CH₂)₄CH₃) and C₆ ((CH₂)₅CH₃). “Branched” means that at least one carbon branch point is present in the group. For example, tert-butyl and isopropyl are both branched groups. Examples of C₁-C₆ alkyl groups include methyl, ethyl, propyl, butyl, 2-methyl-1-propyl, 2-methyl-2-propyl, 2-methyl-1-butyl, 3 methyl-1-butyl, 2-methyl-3-butyl, 2,2-dimethyl-1-propyl, 2-methyl-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, tert-butyl, n-pentyl, isopentyl, neopentyl and n-hexyl. This also applies for C₁-C₆ alkylene which has the meaning as follows: C₁ (CH₂), C₂ (CH₂CH₂), C₃ ((CH₂)₃), C₄ ((CH₂)₄), C₅ ((CH₂)₅) and C₆ ((CH₂)₆).

The term “conjugate” means a nucleic acid, preferably a double-stranded nucleic acid that preferably comprises at least two stretches of RNA and is conjugated to a ligand portion. In this disclosure, a reference to a nucleic acid should also be seen as a reference to a conjugate, since a conjugate comprises a nucleic acid. Therefore, when a disclosure is directed at a nucleic acid or more specifically to an RNA herein, it should be understood that this disclosure is also directed at a conjugate that comprises such a nucleic acid or RNA.

The term “conjugated exclusively to the 3′ and/or 5′ ends” means that the ligand may only be conjugated to the 3′ ends and/or the 5′ ends of one or both RNA strands, and excludes the possibility for the ligand to be conjugated to the oligonucleotide chain at any other location e.g. to a base.

The term “ligand” or “targeting ligand” refers to a moiety (or several moieties) such as a saccharide, such as a galactosamine derivative e.g. GalNAc which may be selected to have an affinity for at least one type of receptor on a target cell. In particular, the receptor is on the surface of a mammalian liver cell, for example, the hepatic asialoglycoprotein receptor (ASGPR).

The term “monomeric ligand” means a ligand comprising only a single moiety which has affinity for at least one type of receptor on a target cell e.g. a single monosaccharide e.g. a single galactosamine derivative (e.g. GalNAc) moiety.

The term “nucleic acid” refers to RNA which forms a double stranded RNA, in particular, siRNA. In the context of this disclosure, RNA should be interpreted as also encompassing nucleic acids that comprise or consist of nucleotides that are 2′ modified, such as 2′-OMe or 2′-F modified.

The term “linker” refers to any moiety which connects an RNA strand to a targeting ligand.

The term “serinol-derived linker moiety” means the linker moiety comprises the following structure:

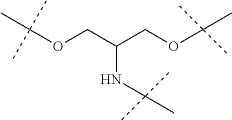

An O atom of said structure typically links to an RNA strand and the N atom typically links to the targeting ligand.

The moiety may comprise other groups such as methyl groups, such as a methyl group, for example a methyl group in the alpha-position:

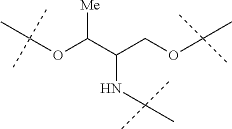

The moiety may comprise a further linker group such as group L defined below, interposed between the N atom of the serinol-derived linker moiety and the targeting ligand. A further linker may also be present interposed between an O atom of the serinol-derived linker moiety and the RNA strand.

The term “treat” or “treating” or “treatment” may include prophylaxis and means to ameliorate, alleviate symptoms, eliminate the causation of the symptoms either on a temporary or permanent basis, or to prevent or slow the appearance of symptoms of the named disorder or condition. The compounds of the invention are useful in the treatment of humans and non-human animals.

By “effective amount” or “therapeutically effective amount” or “effective dose” is meant that amount sufficient to elicit the desired pharmacological or therapeutic effects, thus resulting in effective prevention or treatment of the disorder. Prevention of the disorder is manifested by delaying the onset of the symptoms of the disorder to a medically significant extent. Treatment of the disorder is manifested by a decrease in the symptoms associated with the disorder or an amelioration of the reoccurrence of the symptoms of the disorder.

A “pharmaceutical composition” or “composition” means a mixture of substances suitable for administering to an individual. For example, a pharmaceutical composition can comprise one or more active agents and a pharmaceutical carrier e.g. a sterile aqueous solution.

The term “alternating” as described herein means to occur one after another in a regular way. In other words, alternating means to occur in turn repeatedly. For example if one nucleotide is modified, the next contiguous nucleotide is not modified and the following contiguous nucleotide is modified and so on. One nucleotide may be modified with a first modification, the next contiguous nucleotide may be modified with a second modification and the following contiguous nucleotide is modified with the first modification and so on, where the first and second modifications are different.

The term “inhibit”, “down-regulate”, or “reduce” with respect to gene expression means the expression of the gene, or level of RNA molecules or equivalent RNA molecules encoding one or more proteins or protein subunits (e.g., mRNA), or activity of one or more proteins or protein subunits, is reduced below that observed in the absence of a nucleic acid of the invention; for example the expression may be reduced to 90%, 80%, 70%, 60%, 50%, 40%, 30%, 20%, 10%, 5% or less than that observed in the absence of an inhibitor.

The term “first RNA strand” as used herein means the antisense strand or the A strand, and are used interchangeably throughout the application.

The term “second RNA strand” as used herein means the sense strand or B strand, and are used interchangeably throughout the application.

The present invention relates to a conjugate for inhibiting expression of a target gene in a cell, preferably a hepatic cell or a hepatocyte, said conjugate comprising a nucleic acid portion and ligand portions, said nucleic acid portion comprising at least one duplex region that comprises at least a portion of a first RNA strand and at least a portion of a second RNA strand that is at least partially complementary to the first strand, wherein said first strand is at least partially complementary to at least a portion of RNA transcribed from said target gene, said ligand portions comprising a linker moiety and a targeting ligand for in vivo targeting of cells and being conjugated exclusively to the 3′ and/or 5′ ends of one or both RNA strands, wherein the 5′ end of the first RNA strand is not conjugated, wherein:
(i) the second RNA strand is conjugated at the 5′ end to the targeting ligand, and wherein (a) the second RNA strand is also conjugated at the 3′ end to the targeting ligand and the 3′ end of the first RNA strand is not conjugated; or (b) the first RNA strand is conjugated at the 3′ end to the targeting ligand and the 3′ end of the second RNA strand is not conjugated; or (c) both the second RNA strand and the first RNA strand are also conjugated at the 3′ ends; or
(ii) both the second RNA strand and the first RNA strand are conjugated at the 3′ ends and the 5′ end of the second RNA strand is not conjugated.

The present invention also relates to a conjugate for inhibiting expression of a target gene in a cell, said conjugate comprising a nucleic acid portion and ligand portions, said nucleic acid portion comprising at least one duplex region that comprises at least a portion of a first RNA strand and at least a portion of a second RNA strand that is at least partially complementary to the first strand, wherein said first strand is at least partially complementary to at least a portion of RNA transcribed from said target gene, said ligand portions comprising a linker moiety and a targeting ligand for in vivo targeting of cells and being conjugated exclusively to the 3′ and/or 5′ ends of one or both RNA strands, wherein the 5′ end of the first RNA strand is not conjugated, wherein:
(i) the second RNA strand is conjugated at the 5′ end to the targeting ligand, and wherein (a) the second RNA strand is also conjugated at the 3′ end to the targeting ligand and the 3′ end of the first RNA strand is not conjugated; or (b) the first RNA strand is conjugated at the 3′ end to the targeting ligand and the 3′ end of the second RNA strand is not conjugated; or (c) both the second RNA strand and the first RNA strand are also conjugated at the 3′ ends to the targeting ligand; or
(ii) both the second RNA strand and the first RNA strand are conjugated at the 3′ ends to the targeting ligand and the 5′ end of the second RNA strand is not conjugated; and wherein the first strand of the nucleic acid includes modified nucleotides at a plurality of positions, and wherein the nucleotides at positions 2 and 14 from the 5′ end of the first strand are not modified with a 2′-OMe modification.

The present invention also includes a conjugate for inhibiting expression of a TMPRSS6 gene in a cell, said conjugate comprising a nucleic acid portion and ligand portions, said nucleic acid portion comprising at least one duplex region that comprises at least a portion of a first RNA strand and at least a portion of a second RNA strand that is at least partially complementary to the first strand, wherein said first strand is at least partially complementary to at least a portion of RNA transcribed from said TMPRSS6 gene, said ligand portions a linker moiety, preferably a serinol-derived linker moiety, and a targeting ligand for in vivo targeting of cells and being conjugated exclusively to the 3′ and/or 5′ ends of one or both RNA strands, wherein the 5′ end of the first RNA strand is not conjugated, wherein:
(i) the second RNA strand is conjugated at the 5′ end to the targeting ligand, and wherein (a) the second RNA strand is also conjugated at the 3′ end to the targeting ligand and the 3′ end of the first RNA strand is not conjugated; and
(ii) wherein said first strand includes modified nucleotides at a plurality of positions, and wherein the nucleotides at positions 2 and 14 from the 5′ end of the first strand are not modified with a 2′-OMe modification and the second strand positions opposite first strand positions 11, 12, and 13 (corresponding to second strand positions 7, 8, and 9 from the 5′ end in a 19-mer with two blunt ends) are not modified with 2′-OMe modification.

Optionally, the first RNA strand may comprise the nucleotide sequence of X0371A and/or the second RNA strand may comprise the nucleotide sequence of X0371B.

The target gene may be Factor VI, Eg5, PCSK9, TPX2, apoB, SAA, TTR, RSV, PDGF beta gene, Erb-B gene, Src gene, CRK gene, GRB2 gene, RAS gene, MEKK gene, JNK gene, RAF gene, Erkl/2 gene, PCNA(p21) gene, MYB gene, JU gene, FOS gene, BCL-2 gene, hepcidin, Activated Protein C, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase alpha gene, mutations in the p73 gene, mutations in the p21(WAF I/CIPI) gene, mutations in the p27(KIPI) gene, mutations in the PPM ID gene, mutations in the RAS gene, mutations in the caveolin I gene, mutations in the MIB I gene, mutations in the MTAI gene, mutations in the M68 gene, mutations in tumor suppressor genes, and mutations in the p53 tumor suppressor gene. In particular, the target gene may be TMPRSS6 or ALDH2.

In one embodiment, the TMPRSS6 gene is a human TMPRSS6 gene.

In one embodiment, the target gene is a gene other than: LPA and/or a complement component gene (genes that encode proteins of the immune system's complement system or pathway) and/or ALDH2 and/or TMPRSS6 and/or TTR.

The invention also relates to any first strand or any second strand of nucleic acid as disclosed herein, particularly to a conjugate with such a strand, which comprises no more than 2 base changes when compared to the specific sequence ID provided. For example, one base may be changed within any sequence.

The nucleic acids or conjugates described herein may be capable of inhibiting the expression of the target gene in a cell. The nucleic acid described herein may be capable of partially inhibiting the expression of the target gene in a cell. Inhibition may be complete, i.e. 0% of the expression level of target gene expression in the absence of the nucleic acid of the invention. Inhibition of target gene expression may be partial, i.e. it may be 15%, 20%, 30%, 40%, 50%, 60%, 70%, 75%, 80%, 85%, 90%, 95% of target gene expression in the absence of a nucleic acid of the invention. Inhibition may last 4 weeks, 5 weeks, 6 weeks, 7 weeks, 8 weeks, 10 weeks, 11 weeks, 12 weeks, 13 weeks, 14 weeks or up to 3 months, when used in a subject, such as a human subject. The nucleic acid or conjugated nucleic acid or composition comprising the same may be for use once, every week, every two weeks, every three weeks, every four weeks, every five weeks, every six weeks, every seven weeks, or every eight weeks. The nucleic acid or conjugated nucleic acid may be for use subcutaneously or intravenously.

In cells and/or subjects treated with or receiving a nucleic acid or conjugated nucleic acid of the present invention, the target gene expression may be inhibited compared to untreated cells and/or subjects by at least about 30%, 35%, 40%, 45%, 50%, 55%, 60%, 65%, 70%, 75%, 80%, 85%, 90%, 95%, 98%, or 100%. The level of inhibition may allow treatment of a disease associated 15 with target gene expression or overexpression, or may allow further investigation into the functions of the target gene product. The inhibition is preferably mediated through RNAi.

In one embodiment, the conjugates are for inhibiting expression of a target gene in a hepatic cell or more specifically a hepatocyte. In such a case, the targeting ligands are preferably GalNAc 20 ligands. The conjugates are preferably for use in the treatment of a disease, preferably for a disease that can be treated by targeting a gene target in the liver, and more specifically, by targeting a gene target in hepatocytes.

The linker may be a serinol-derived linker moiety.

In an embodiment of the present invention, the second RNA strand (i.e. the sense strand) is conjugated at the 5′ end to the targeting ligand, the first RNA strand (i.e. the antisense strand) is conjugated at the 3′ end to the targeting ligand and the 3′ end of the second RNA strand (i.e. the sense strand) is not conjugated, such that a conjugate with the following schematic structure is formed:

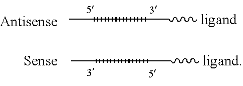

In an embodiment of the present invention, the second RNA strand (i.e. the sense strand) is conjugated at the 5′ end to the targeting ligand, the second RNA strand (i.e. the sense strand) is 35 also conjugated at the 3′ end to the targeting ligand and the 3′ end of the first RNA strand (i.e. the antisense strand) is not conjugated, such that a conjugate with the following schematic structure is formed:

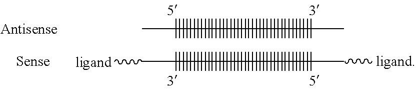

In an embodiment of the present invention, both the second RNA strand (i.e. the sense strand) and the first RNA strand (i.e. the antisense strand) are conjugated at the 3′ ends to the targeting ligand and the 5′ end of the second RNA strand (i.e. the sense strand) is not conjugated, such that a conjugate with the following schematic structure is formed:

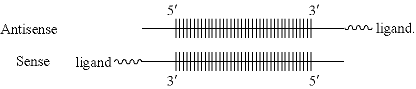

In an embodiment of the present invention, the second RNA strand (i.e. the sense strand) is conjugated at the 5′ end to the targeting ligand and both the second RNA strand (i.e. the sense strand) and the first RNA strand (i.e. the antisense strand) are also conjugated at the 3′ ends to the targeting ligand, such that a conjugate with the following schematic structure is formed:

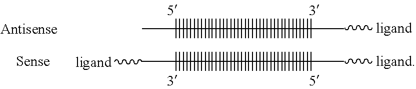

In any one of the above embodiments, v indicates the linker which conjugates the ligand to the ends of the nucleic acid portion; the ligand may be a GalNAc moiety such as GalNAc; and wherein

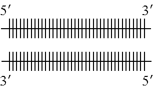

represents the nucleic acid portion.

These schematic diagrams are not intended to limit the number of nucleotides in the first or second strand, nor do the diagrams represent any kind of limitation on complementarity of the bases or any other limitation.

The ligands may be monomeric or multimeric (e.g. dimeric, trimeric, etc.).

Suitably, the ligands are monomeric, thus containing a single targeting ligand moiety, e.g. a single GalNAc moiety.

Alternatively, the ligands may be dimeric ligands wherein the ligand portions comprise two linker moieties, such as serinol-derived linker moieties or non-serinol linker moieties, each linked to a single targeting ligand moiety.

The ligands may be trimeric ligands wherein the ligand portions comprise three linker moieties, such as serinol-derived linker moieties or non-serinol linker moieties, each linked to a single targeting ligand moiety.

The two or three serinol-derived linker moieties may be linked in series e.g. as shown below:

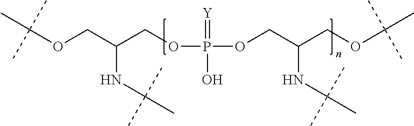

wherein n is 1 or 2 and Y is S or 0.

Preferably, the ligands are monomeric.

Suitably, the conjugated RNA strands are conjugated to a targeting ligand via a linker moiety including a further linker wherein the further linker is or comprises a saturated, unbranched or branched C₁₋₁₅ alkyl chain, wherein optionally one or more carbons (for example 1, 2 or 3 carbons, suitably 1 or 2, in particular 1) is/are replaced by a heteroatom selected from O, N, S(O)ₚ, wherein p is 0, 1 or 2 (for example a CH₂ group is replaced with 0, or with NH, or with S, or with SO₂ or a —CH₃ group at the terminus of the chain or on a branch is replaced with OH or with NH₂) wherein said chain is optionally substituted by one or more oxo groups (for example 1 to 3, such as 1 group).

Suitably, the linker moiety is a serinol-derived linker moiety.

More suitably, the further linker comprises a saturated, unbranched C₁₋₁₅ alkyl chain wherein one or more carbons (for example 1, 2 or 3 carbons, suitably 1 or 2, in particular 1) is/are replaced by an oxygen atom. 30 More suitably, the further linker comprises a PEG-chain.

More suitably, the further linker comprises a saturated, unbranched C₁₋₁₅ alkyl chain.

More suitably, the further linker comprises a saturated, unbranched C₁₋₆ alkyl chain.

More suitably, the further linker comprises a saturated, unbranched C₄ or C₆ alkyl chain, e.g. a C₄ alkyl chain.

In an embodiment, [symbol] is a linking moiety of formula (I):

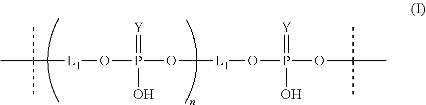

wherein n, Y and L₁ are defined below and the O of the phosphoro-group is attached to the terminal oligonucleoside of the RNA strands.

Thus in an embodiment, the targeting ligand portion is a linking moiety of formula (II):

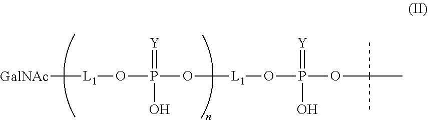

wherein n, Y and L₁ are defined below and the O of the phosphoro-group is attached to the terminal oligonucleoside of the RNA strands.

Suitably, [symbol] is a linking moiety of formula (III):

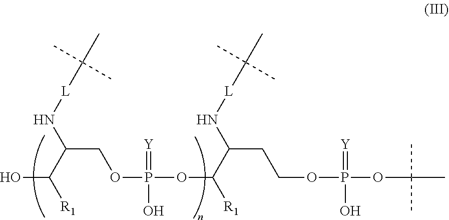

wherein n, Y, R₁ and L are defined below, L is connected to the targeting ligand e.g. GalNAc and the O of the phosphoro-group is attached to the terminal oligonucleoside of the RNA strands.

Suitably, the targeting ligand portion is a linking moiety of formula (IV):

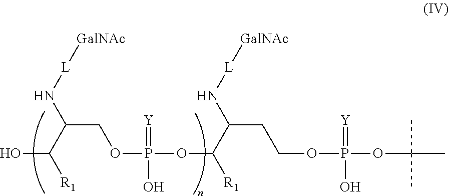

wherein n, Y, R₁ and L are defined below and the O of the phosphoro-group is attached to the terminal oligonucleoside of the RNA strands.

Suitably, [symbol] is a linking moiety of formula (V):

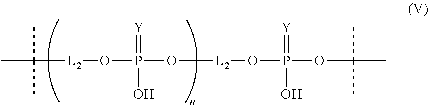

wherein n, Y and L₂ are defined below and the O of the phosphoro-group is attached to the terminal oligonucleoside of the RNA strands.

Suitably, the targeting ligand portion is a linking moiety of formula (VI):

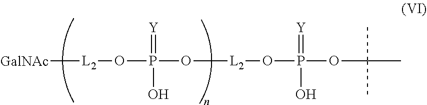

wherein n, Y and L₂ are defined below and the O of the phosphoro-group is attached to the terminal oligonucleoside of the RNA strands.

Suitably, [symbol] is a linking moiety of formula (VII):

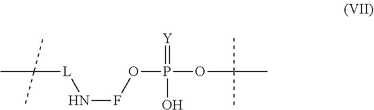

wherein F, Y and L are defined below and the O of the phosphoro-group is attached to the terminal oligonucleoside of the RNA strands.

Suitably, the targeting ligand portion is a linking moiety of formula (VIII):

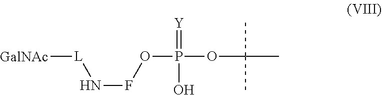

wherein F, Y and L are defined below and the O of the phosphoro-group is attached to the terminal oligonucleoside of the RNA strands.

Suitably, L is

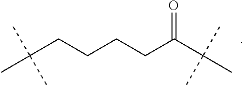

In any of the above structures, suitably the ligands are selected from GalNAc and galactose moieties, especially GalNAc moieties. Alternatively, GalNac may be replaced by another targeting ligand, e.g. a saccharide.

In an embodiment of the invention, the first RNA strand is a compound of formula (IX):

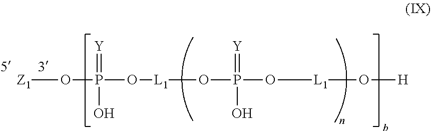

wherein b is preferably 0 or 1; and

the second RNA strand is a compound of formula (X):

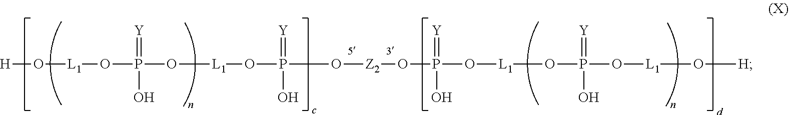

wherein:
c and d are independently preferably 0 or 1;
Z₁ and Z₂ are the RNA portions of the first and second RNA strands respectively;
Y is independently O or S;
n is independently 0, 1, 2 or 3; and
L₁ is a linker to which a ligand is attached, wherein L₁ is preferably the same or different in formulae (IX) and (X), and is the same or different within formulae (IX) and (X) when L₁ is present more than once within the same formula, wherein L₁ is preferably of formula (XI);

and wherein b+c+d is 2 or 3.

Preferably, L₁ in formulae (IX) and (X) or in any other formula, is of formula (XI):

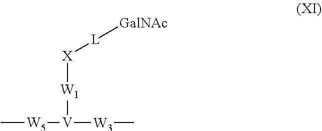

wherein:
L is selected from the group comprising, or preferably consisting of:
   —(CH₂)ᵣC(O)—, wherein r=2-12;
   —(CH₂—CH₂—O)ₛ—CH₂—C(O)—, wherein s=1-5;
   —(CH₂)ₜ—CO—NH—(CH₂)ₜ—NH—C(O)—, wherein t is independently 1-5;
   —(CH₂)ᵤ—CO—NH—(CH₂)ᵤ—C(O)—, wherein u is independently 1-5; and
   —(CH₂)ᵥ—NH—C(O)—, wherein v is 2-12; and
wherein the terminal C(O), if present, is attached to X of formula (XI), or if X is absent, to W₁ of formula (XI), or if W₁ is also absent, to V of formula (XI);
W₁, W₃ and W₅ are individually absent or selected from the group comprising, or preferably consisting of:
   —(CH₂)ᵣ, wherein r=1-7;
   —(CH₂)ₛ—O—(CH₂)ₛ—, wherein s is independently 0-5;
   —(CH₂)ₜ—S—(CH₂)ₜ—, wherein t is independently 0-5;
X is absent or is selected from the group comprising, or preferably consisting of: NH, NCH₃ or NC₂H₅;
V is selected from the group comprising, or preferably consisting of: CH, N,

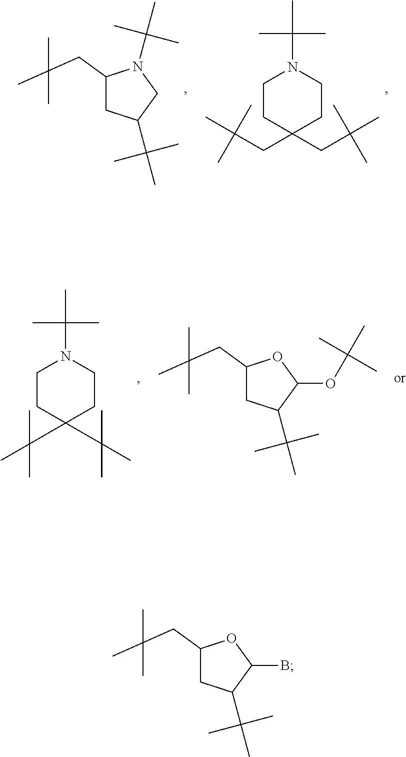

wherein B, if present, is a modified or natural nucleobase.

In one aspect, the first strand is a compound of formula (XII)

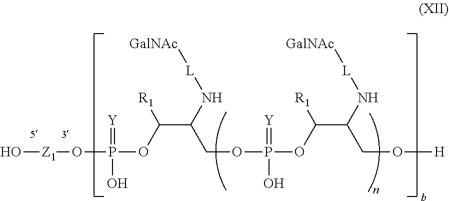

wherein b is preferably 0 or 1; and

the second strand is a compound of formula (XII):

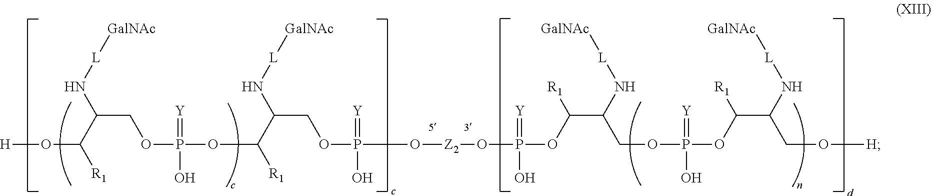

wherein c and d are independently preferably 0 or 1;

wherein:
Z₁ and Z₂ are respectively the first and second strand of the nucleic acid;
Y is independently O or S;
R₁ is H or methyl;
n is independently preferably 0, 1, 2 or 3; and
L is the same or different in formulae (XII) and (XIII), and is the same or different within formulae (XII) and (XIII) when L is present more than once within the same formula, and is selected from the group comprising, or preferably consisting of:
   —(CH₂)ᵣC(O)—, wherein r=2-12;
   —(CH₂—CH₂—O)ₛ—CH₂—C(O)—, wherein s=1-5;
   —(CH₂)ₜ—CO—NH—(CH₂)ₜ—NH—C(O)—, wherein t is independently 1-5;
   —(CH₂)ᵤ—CO—NH—(CH₂)ᵤ—C(O)—, wherein u is independently 1-5; and
   —(CH₂)ᵥ—NH—C(O)—, wherein v is 2-12; and wherein the terminal C(O), if present, is attached to the NH group (of the linker, not of the targeting ligand);

and wherein b+c+d is preferably 2 or 3.

Suitably, the first RNA strand is a compound of formula (XIV):

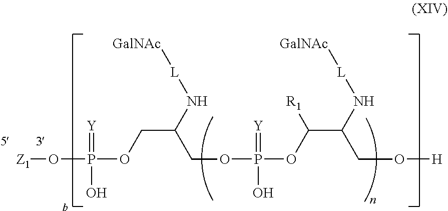

wherein b is 0 or 1; and

the second RNA strand is a compound of formula (XV):

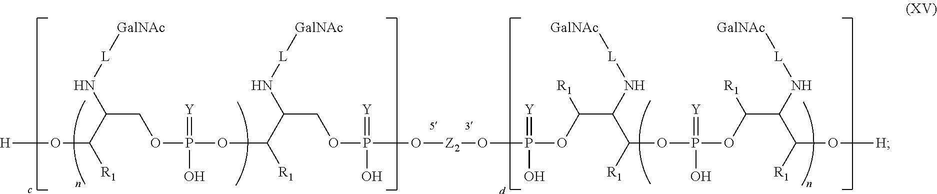

wherein c and d are independently 0 or 1;

wherein:
Z₁ and Z₂ are the RNA portions of the first and second RNA strands respectively;
Y is O or S;
R₁ is H or methyl;
n is 0, 1, 2 or 3; and
L is the same or different in formulae (XIV) and (XV) and is selected from the group consisting of:
   —(CH₂)ᵣC(O)—, wherein r=2-12;
   —(CH₂—CH₂—O)ₛ—CH₂—C(O)—, wherein s=1-5;
   —(CH₂)ₜ—CO—NH—(CH₂)ₜ—NH—C(O)—, wherein t is independently is 1-5;
   —(CH₂)ᵤ—CO—NH—(CH₂)ᵤ—C(O)—, wherein u is independently is 1-5; and
   —(CH₂)ᵥ—NH—C(O)—, wherein v is 2-12; and
wherein the terminal C(O) (if present) is attached to the NH group;

and wherein b+c+d is 2 or 3.

Suitably, the first RNA strand is a compound of formula (XVI):

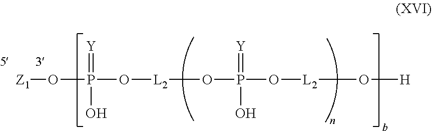

wherein b is 0 or 1; and

the second RNA strand is a compound of formula (XVII):

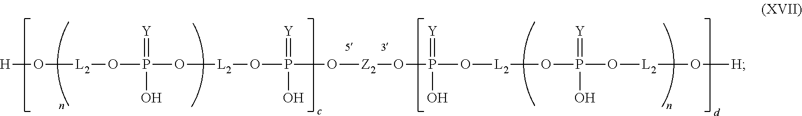

wherein:
c and d are independently 0 or 1;
Z₁ and Z₂ are the RNA portions of the first and second RNA strands respectively;
Y is O or S;
n is 0, 1, 2 or 3; and
L₂ is the same or different in formulae (XVI) and (XVII) and is the same or different in moieties bracketed by b, c and d, and is selected from the group consisting of:

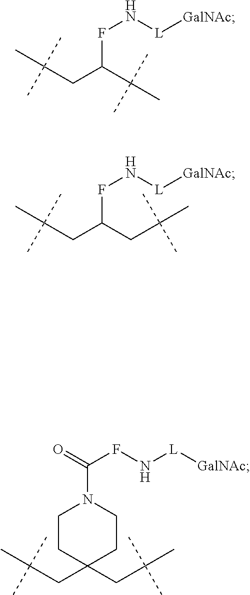

and optionally

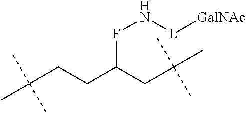

and optionally

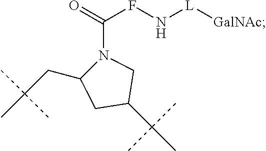

or
n is 0 and L₂ is:

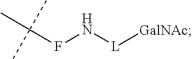

and the terminal OH group is absent such that the following moiety is formed:

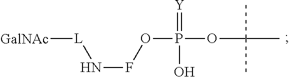

wherein
F is a saturated branched or unbranched (such as unbranched) C₁₋₈alkyl (e.g. C₁₋₆alkyl) chain wherein one of the carbon atoms is optionally replaced with an oxygen atom provided that said oxygen atom is separated from another heteroatom (e.g. an O or N atom) by at least 2 carbon atoms;
L is the same or different in formulae (XVI and (XVII) and is selected from the group consisting of:
   —(CH₂)ᵣC(O)—, wherein r=2-12;
   —(CH₂—CH₂—O)ₛ—CH₂—C(O)—, wherein s=1-5;
   —(CH₂)ₜ—CO—NH—(CH₂)ₜ—NH—C(O)—, wherein t is independently is 1-5;
   —(CH₂)ᵤ—CO—NH—(CH₂)ᵤ—C(O)—, wherein u is independently is 1-5; and
   —(CH₂)ᵥ—NH—C(O)—, wherein v is 2-12; and
wherein the terminal C(O) (if present) is attached to the NH group;

and wherein b+c+d is 2 or 3.

Suitably, b is 0, c is 1 and d is 1; b is 1, c is 0 and d is 1; b is 1, c is 1 and d is 0; or b is 1, c is 1 and d is 1. More suitably, b is 0, c is 1 and d is 1; b is 1, c is 0 and d is 1; or b is 1, c is 1 and d is 1. Most suitably, b is 0, c is 1 and d is 1.

In one embodiment, Y is O. In another embodiment, Y is S.

In one embodiment, R₁ is H or methyl. In one embodiment, R₁ is H. In another embodiment, R₁ is methyl.

In one embodiment, n is 0, 1, 2 or 3. Suitably, n is 0.

In one embodiment, L is selected from the group consisting of:
—(CH₂)ᵣC(O)—, wherein r=2-12;
—(CH₂—CH₂—O)ₛ—CH₂—C(O)—, wherein s=1-5;
—(CH₂)ₜ—CO—NH—(CH₂)ₜ—NH—C(O)—, wherein t is independently is 1-5;
—(CH₂)ᵤ—CO—NH—(CH₂)ᵤ—C(O)—, wherein u is independently is 1-5; and
—(CH₂)ᵥ—NH—C(O)—, wherein v is 2-12;
wherein the terminal C(O) is attached to the NH group.

Suitably, L is —(CH₂)ᵣC(O)—, wherein r=2-12. Suitably, r=2-6. More suitably, r=4 or 6 e.g. 4.

Suitably, L is:

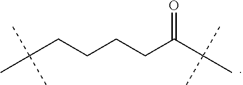

Example F moieties include (CH₂)₁₋₆ e.g. (CH₂)₁₋₄ e.g. CH₂, (CH₂)₄, (CH₂)₅ or (CH₂)₆, or CH₂O(CH₂)₂₋₃, e.g. CH₂O(CH₂)CH₃.

Suitably, L₂ is:

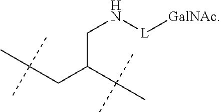

Suitably, L₂ is:

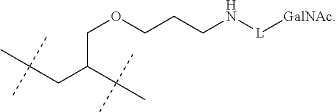

Suitably, L₂ is:

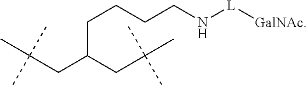

Suitably, L₂ is:

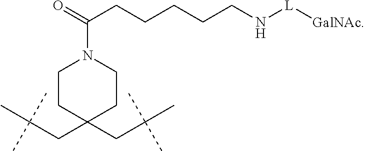

Suitably, n is 0 and L₂ is:

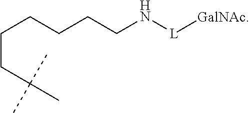

and the terminal OH group is absent such that the following moiety is formed:

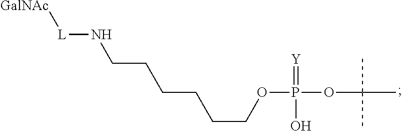

wherein Y is as defined elsewhere herein.

Within the moiety bracketed by b, c and d, L₂ is typically the same. Between moieties bracketed by b, c and d, L₂ may be the same or different. In an embodiment, L₂ in the moiety bracketed by c is the same as the L₂ in the moiety bracketed by d. In an embodiment, L₂ in the moiety bracketed by c is not the same as L₂ in the moiety bracketed by d. In an embodiment, the L₂ in the moieties bracketed by b, c and d is the same, for example when the linker moiety is a serinol-derived linker moiety.

Serinol derived linker moieties may be based on serinol in any stereochemistry i.e. derived from L-serine isomer, D-serine isomer, a racemic serine or other combination of isomers. In a preferred aspect of the invention, the serinol-GalNAc moiety (SerGN) has the following stereochemistry:

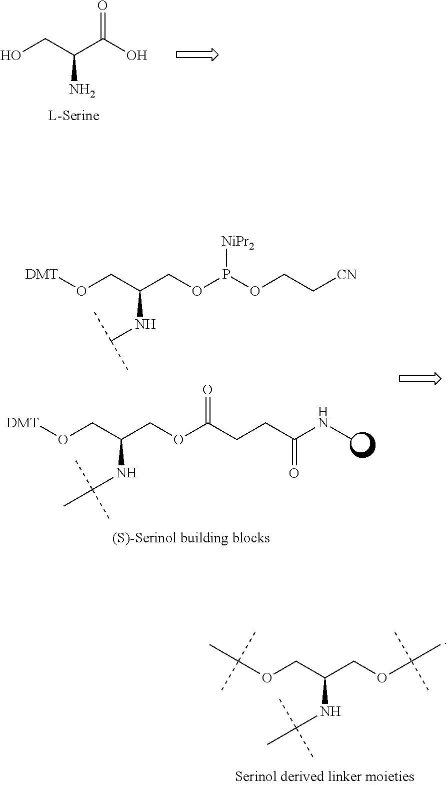

i.e. is based on an (S)-serinol-amidite or (S)-serinol succinate solid supported building block derived from L-serine isomer.

In one embodiment, the targeted cells are hepatocytes.

In one aspect, b is 0, c is 1 and d is 1; b is 1, c is 0 and d is 1; b is 1, c is 1 and d is 0; or b is 1, c is 1 and d is 1 in any of the nucleic acids of formulae (IX) and (X) or (XII) and (XIII) or (XIV) and (XV) or (XVI) and (XVII). Preferably, b is 0, c is 1 and d is 1; b is 1, c is 0 and d is 1; or b is 1, c is 1 and d is 1. Most preferably, b is 0, c is 1 and d is 1.

In one aspect, Y is O in any of the nucleic acids of formulae (IX) and (X) or (XII) and (XIII) or (XIV) and (XV) or (XVI) and (XVII). In another aspect, Y is S. In a preferred aspect, Y is independently selected from O or S in the different positions in the formulae.

In one aspect, R₁ is H or methyl in any of the nucleic acids of formulae (XII) and (XIII) or (XIV) and (XV). In one aspect, R₁ is H. In another aspect, R₁ is methyl.

In one aspect, n is 0, 1, 2 or 3 in any of the nucleic acids of formulae (IX) and (X) or (XII) and (XIII) or (XIV) and (XV) or (XVI) and (XVII). Preferably, n is 0.

Examples of F moieties in any of the nucleic acids of formulae (XVI) and (XVII) include (CH₂)₁₋₆ e.g. (CH₂)₁-4 e.g. CH₂, (CH₂)₄, (CH₂)₅ or (CH₂)₆, or CH₂O(CH₂)₂₋₃, e.g. CH₂O(CH₂)CH₃.

In a preferred aspect, the first strand of the nucleic acid is a compound of formula (XII) and the second strand of the nucleic acid is a compound of formula (XIII), wherein:
b is 0;
c and d are 1;
n is 0;
Z₁ and Z₂ are respectively the first and second strand of the nucleic acid;
Y is S;
R₁ is H; and
L is —(CH₂)₄—C(O)—, wherein the terminal C(O) of L is attached to the N atom of the linker (ie not a possible N atom of a targeting ligand).

In another preferred aspect, the first strand of the nucleic acid is a compound of formula (IX) and the second strand of the nucleic acid is a compound of formula (X), wherein:
b is 0;
c and d are 1;
n is 0;
Z₁ and Z₂ are respectively the first and second strand of the nucleic acid;
Y is S; and
L₁ is of formula (XI), wherein:
   W₁ is —CH₂—O—(CH₂)₃—;
   W₃ is —CH₂—;
   W₅ is absent;
   V is CH;
   X is NH; and
   L is —(CH₂)₄—C(O)— wherein the terminal C(O) of L is attached to the N atom of X in formula (XI).

In another preferred aspect, the first strand of the nucleic acid is a compound of formula (IX) and the second strand of the nucleic acid is a compound of formula (X), wherein:
b is 0;
c and d are 1;
n is 0;
Z₁ and Z₂ are respectively the first and second strand of the nucleic acid;
Y is S;
L₁ is of formula (XI), wherein:
   W₁, W₃ and W₅ are absent;
   V is

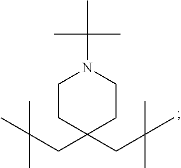 [SMILES: CC(C)(C)CC1(CC(C)(C)C)CCN(C(C)(C)C)CC1]

X is absent; and
L is —(CH₂)₄—C(O)—NH—(CH₂)₅—C(O)—, wherein the terminal C(O) of L is attached to the
N atom of V in formula (XI).

In any one of the above formulae where GalNAc is present, the GalNAc may be substituted for any other targeting ligand, such as those mentioned herein.

Nucleic Acid

In all cases described herein, the nucleic acid is RNA which forms a double stranded RNA. The nucleic acid may be a functional nucleic acid, and in particular, the nucleic acid is an siRNA. The siRNA is able to interfere with or inhibit gene expression, preferably through the iRNA pathway. Inhibition may be complete or partial and results in down regulation of gene expression in a targeted manner. The nucleic acid comprises two separate polynucleotide strands; the first strand, which may also be a guide strand; and a second strand, which may also be a passenger strand. The first strand may also be referred to as an antisense strand or strand A. The second strand may also be referred to as a sense strand or strand B.

The term nucleic acid or RNA can refer in the context of this disclosure only to a nucleic acid or RNA or to a nucleic acid or RNA that is conjugated to one or several ligands. A reference to a nucleic acid or RNA herein usually refers to the nucleic acid or RNA portion of a conjugate of the invention.

The nucleic acid comprises a double stranded nucleic acid portion or duplex region formed by all or a portion of the first strand (also known in the art as a guide strand) and all or a portion of the second strand (also known in the art as a passenger strand). The duplex region is defined as beginning with the first base pair formed between the first strand and the second strand and ending with the last base pair formed between the first strand and the second strand, inclusive.

By duplex region it is meant the region in two complementary or substantially complementary oligonucleotides that form base pairs with one another, either by Watson-Crick base pairing or any other manner that allows for a duplex between oligonucleotide strands that are complementary or substantially complementary. The duplex region of a double stranded RNA may range from 15-30 nucleotide base pairs using the Watson-crick base pairing. The duplex region may have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 base pairs.

Thus, in an embodiment, the nucleic acid portion comprises or consists of two RNA strands of 15-30 based-paired ribonucleotides, suitably 19-25 or 20-25 e.g. 19-23 based-paired ribonucleotides. Preferably, the nucleic acid has 19-25 e.g. 19 to 23 base pairs. For example, the nucleic acid may be 19, 20, 21, 22 or 23 base pairs in length. For example, an oligonucleotide strand having 21 nucleotide units can base pair with another oligonucleotide of 21 nucleotide units, yet only 19 nucleotides on each strand are complementary or substantially complementary, such that the “duplex region” consists of 19 base pairs. The remaining base pairs may exist as 5′ and 3′ overhangs, or as single stranded regions. Overhangs are discussed in more detail below. Further, within the duplex region, 100% complementarity is not required; substantial complementarity is allowable within a duplex region. Substantial complementarity refers to complementarity between the strands such that they are capable of annealing under biological conditions. Techniques to empirically determine if two strands are capable of annealing under biological conditions are well known in the art. Alternatively, two strands can be synthesised and added together under biological conditions to determine if they anneal to one another.

The portion of the first strand and second strand that form at least one duplex region may be fully complementary and are at least partially complementary to each other. Depending on the length of a nucleic acid, a perfect match in terms of base complementarity between the first strand and second strand is not necessarily required. However, the first and second strands must be able to hybridise under physiological conditions. The complementarity may be at least 70%, 75%, 80%, 85%, 90% or 95%.

The first strand and the second strand may each comprise a region of complementarity which comprises at least 15 contiguous nucleotides differing by no more than 3 nucleotides. The first strand and the target may be fully complementary and are at least partially complementary to each other, preferably fully complementary. The complementarity may be at least 70%, 75%, 80%, 85%, 90%, 95% or 100%, preferably 90%, 95% or 100% (e.g. 100%). The first strand and the target may each comprise a region of complementarity which comprises at least 15 contiguous nucleotides differing by no more than 3 nucleotides.

The nucleic acid of the present invention can be produced using routine methods in the art including chemically synthesis or expressing the nucleic acid either in vitro (e.g., run off transcription) or in vivo. For example, using solid phase chemical synthesis or using an expression vector. In one embodiment, the expression vector can produce the nucleic acid of the invention in a target cell. Methods for the synthesis of the nucleic acid molecule described herein are known to persons skilled in the art.

The ribonucleic acid constructs may be incorporated into suitable vector systems. Preferably the vector comprises a promoter for the expression of RNA. The promoter may be selected from any known in the art such as pol Ill, U6, H1 or 7SK.

The portion of the first strand and second strand that form at least one duplex region may be fully complementary and are at least partially complementary to each other.

Depending on the length of a nucleic acid, a perfect match in terms of base complementarity between the first strand and second strand is not necessarily required. However, the first and second strands must be able to hybridise under physiological conditions.

The complementarity between the first strand and second strand in the at least one duplex region may be perfect in that there are no nucleotide mismatches or additional/deleted nucleotides in either strand. Alternatively, the complementarity may not be perfect. The complementarity may be at least 70%, 75%, 80%, 85%, 90% or 95%.

The first strand and the second strand may each comprise a region of complementarity which comprises at least 15 contiguous nucleotides.

The nucleic acid involves the formation of a duplex region between all or a portion of the first strand and a portion of the target nucleic acid. The portion of the target nucleic acid that forms a duplex region with the first strand, defined as beginning with the first base pair formed between the first strand and the target sequence and ending with the last base pair formed between the first strand and the target sequence, inclusive, is the target nucleic acid sequence or simply, target sequence. The duplex region formed between the first strand and the second strand need not be the same as the duplex region formed between the first strand and the target sequence. That is, the second strand may have a sequence different from the target sequence however, the first strand must be able to form a duplex structure with both the second strand and the target sequence.

The complementarity between the first strand and the target sequence may be perfect (no nucleotide mismatches or additional/deleted nucleotides in either nucleic acid).

The complementarity between the first strand and the target sequence may not be perfect. The complementarity may be at least 70%, 80%, 85%, 90% or 95%.

The identity between the first strand and the complementary sequence of the target sequence may be at least 75%, 80%, 85%, 90% or 95%, provided a nucleic acid is capable of reducing or inhibiting the expression of the target gene.

The nucleic acid may be able to reduce expression of the target gene by at least 25%, 50% or 75% of a comparative nucleic acid with perfect identity to the first strand and target sequence.

The nucleic acid may comprise a first strand and a second strand that are each from 17-35 or 19-25 nucleotides in length. The first strand and the second strand may be of different lengths.

The nucleic acid may be 15-25 nucleotide pairs in length. The nucleic acid may be 17-23 nucleotide pairs in length. The nucleic acid may be 17-25 nucleotide pairs in length. The nucleic acid may be 23-24 nucleotide pairs in length. The nucleic acid may be 19-21 nucleotide pairs in length. The nucleic acid may be 21-23 nucleotide pairs in length.

The nucleic acid may comprise a duplex region that consists of 19-25 nucleotide base pairs. The duplex region may consist of 17, 18, 19, 20, 21, 22, 23, 24 or 25 base pairs which may be contiguous.

The nucleic acid may be blunt ended at both ends; have an overhang at one end and a blunt end at the other end; or have an overhang at both ends.

RNA Modifications

Modifications of the RNA molecules of the present invention generally provide a powerful tool in overcoming potential limitations including, but not limited to, in vitro and in vivo stability and bioavailability inherent to native RNA molecules. Modification can further enhance the functional delivery of an siRNA to a target cell. Modified siRNA can also minimize the possibility of activating interferon activity in humans.

The term “modification” indicates a difference from a naturally occurring molecule. The term “modification” does not refer to the conjugation of the present invention, but instead refers to additional modifications which may or may not exist and are described below.

The nucleic acid may be a modified nucleic acid. The modification may be selected from substitutions or insertions with analogues of nucleic acids or bases and chemical modification of the base, sugar or phosphate moieties compared to essentially that which occurs in nature. The modifications that occur in a nucleic acid will be repeated within a polynucleotide molecule such as a modification of a base, or a phosphate moiety, or the non-linking O of a phosphate moiety. A modification may only occur at a 3′ or 5′ terminal position, may only occur in terminal regions, such as at a position on a terminal nucleotide or in the last 2, 3, 4, 5, or 10 nucleotides of a strand. A modification may occur in a double strand region, a single strand region, or in both. A modification may occur only in the double strand region of a nucleic acid of the invention or may only occur in a single strand region of a nucleic acid of the invention. A phosphorothioate modification at a non-linking O position may only occur at one or both termini, may only occur in a terminal region, e.g., at a position on a terminal nucleotide or in the last 2, 3, 4 or 5 nucleotides of a strand, or may occur in duplex and/or in single strand regions, particularly at termini.

Details of possible modifications of the nucleic acid of the conjugates according to the invention are described below.

Modification of Nucleotides

Unmodified polynucleotides, particularly ribonucleotides, may be prone to degradation by cellular nucleases, and, as such, modified nucleotides may be included in the nucleic acid of the invention.

Thus, one or more nucleotides of a nucleic acid of the present invention (i.e. a nucleic acid of a conjugate of the present invention) may be modified. One or more nucleotides on the first and/or second strand may be modified, to form modified nucleotides. One or more of the odd numbered nucleotides of the first strand may be modified. One or more of the even numbered nucleotides of the first strand may be modified. One or more of the even numbered nucleotides of the first strand may be modified by at least a second modification, wherein the at least second modification is different from the modification on the one or more odd nucleotides. At least one of the one or more modified even numbered nucleotides may be adjacent to at least one of the one or more modified odd numbered nucleotides.

A plurality of odd numbered nucleotides in the first strand may be modified in the nucleic acid of the invention. A plurality of even numbered nucleotides in the first strand may be modified in the nucleic acid of the invention. A plurality of even numbered nucleotides in the first strand may be modified by a second modification. The first strand may comprise adjacent nucleotides that are modified by a common modification. The first strand may also comprise adjacent nucleotides that are modified by a second different modification.

One or more of the odd numbered nucleotides of the second strand may be modified by a modification that is different to the modification of the odd numbered nucleotides on the first strand and/or one or more of the even numbered nucleotides of the second strand. One or more of the even numbered nucleotides of the second strand may be modified by a modification that is different to the modification of the odd numbered nucleotides on the first strand and/or one or more of the even numbered nucleotides of the second strand. At least one of the one or more modified even numbered nucleotides of the second strand may be adjacent to the one or more modified odd numbered nucleotides. A plurality of odd numbered nucleotides of the second strand may be modified by a common modification and/or a plurality of even numbered nucleotides may be modified by the same modification that is present on the first strand odd numbered nucleotides. A plurality of odd numbered nucleotides on the second strand may be modified by a second modification, wherein the second modification is different from the modification of the first strand odd numbered nucleotides.

One or more or each of the odd numbered nucleotides may be modified in the first strand and one or more or each of the even numbered nucleotides may be modified in the second strand. One or more or each of the even numbered nucleotides may be modified in the first strand and one or more or each of the even numbered nucleotides may be modified in the second strand. One or more or each of the odd numbered nucleotides may be modified in the first strand and one or more of the odd numbered nucleotides may be modified in the second strand by a common modification. One or more or each of the even numbered nucleotides may be modified in the first strand and one or more or each of the odd numbered nucleotides may be modified in the second strand by a common modification. In any of the above embodiments, one or more or each of the alternating nucleotides on either or both strands may be modified by a second modification.

The second strand comprises adjacent nucleotides that are modified by a common modification, which may be a second modification that is different from the modification of the odd numbered nucleotides of the first strand.

In the nucleic acid of the invention, each of the odd numbered nucleotides in the first strand and each of the even numbered nucleotides in the second strand may be modified with a common modification and, each of the even numbered nucleotides may be modified in the first strand with a second modification and each of the odd numbered nucleotides may be modified in the second strand with a second different modification.

The nucleic acid of the invention may have the modified nucleotides of the first strand shifted by at least one nucleotide relative to the unmodified or differently modified nucleotides of the second strand. Modifications to the nucleotides are discussed under “Modifications to sugar moiety” below.

The modified nucleotide may be in the duplex region. The modified nucleotide may be outside the duplex region, i.e., in a single stranded region. The modified nucleotide may be on the first strand and may be outside the duplex region. The modified nucleotide may be on the second strand and may be outside the duplex region. The 3′-terminal nucleotide of the first strand may be a modified nucleotide. The 3′-terminal nucleotide of the second strand may be a modified nucleotide. The 5′-terminal nucleotide of the first strand may be a modified nucleotide. The 5′-terminal nucleotide of the second strand may be a modified nucleotide.

An nucleic acid of the invention may have 1 modified nucleotide or a nucleic acid of the invention may have about 2-4 modified nucleotides, or a nucleic acid may have about 4-6 modified nucleotides, about 6-8 modified nucleotides, about 8-10 modified nucleotides, about 10-12 modified nucleotides, about 12-14 modified nucleotides, about 14-16 modified nucleotides about 16-18 modified nucleotides, about 18-20 modified nucleotides, about 20-22 modified nucleotides, about 22-24 modified nucleotides, 24-26 modified nucleotides or about 26-28 modified nucleotides. In each case the nucleic acid comprising said modified nucleotides retains at least 50% of its activity as compared to the same nucleic acid but without said modified nucleotides. The nucleic acid may retain 55%, 60%, 65%, 70%, 75%, 80%, 85%, 90%, 95% or 100% or above of its activity as compared to the same nucleic acid but without said modified nucleotides.

The modified nucleotide may comprise a purine or a pyrimidine base. At least half of the purines may be modified. At least half of the pyrimidines may be modified. All of the purines may be modified. All of the pyrimidines may be modified.

The nucleic acid may comprise a nucleotide comprising a modified nucleotide selected from 2-aminoadenosine, 2,6-diaminopurine riboside, inosine, pyridin-4-one riboside, pyridin-2-one riboside, phenyl riboside, pseudouridine, 2,4,6-trimethoxy benzene riboside, 3-methyl uridine, dihydrouridine, naphthyl, aminophenyl riboside, 5-alkylcytidine (e.g., 5-methylcytidine), 5-alkyluridine (e.g., ribothymidine), 5-halouridine (e.g., 5-bromouridine), 6-azapyrimidine riboside, 6-alkylpyrimidine riboside (e.g. 6-methyluridine), propyne riboside (e.g. 5-(1-propynyl)-2′-deoxy-Uridine (pdU) or 5-(1-propynyl)-2′-deoxyCytidine (pdC)), queuosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueuosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueuosine, uridine-5-oxyacetic acid and 2-thiocytidine.

One or more of the odd numbered nucleotides of the first strand of the nucleic acid of the invention may be modified wherein the first strand is numbered 5′ to 3′. The term “odd numbered” as described herein means a number not divisible by two. Examples of odd numbers are 1, 3, 5, 7, 9, 11 and so on. One or more of the even numbered nucleotides of the first strand of the nucleic acid of the invention may be modified, wherein the first strand is numbered 5′ to 3′. The term “even numbered” as described herein means a number which is evenly divisible by two. Examples of even numbers are 2, 4, 6, 8, 10, 12, 14 and so on. One or more of the odd numbered nucleotides of the second strand of the nucleic acid of the invention may be modified wherein the second strand is numbered 3′ to 5′. One or more of the even numbered nucleotides of the second strand of the nucleic acid of the invention may be modified, wherein the second strand is numbered 3′ to 5′.

One or more nucleotides on the first and/or second strand may be modified, to form modified nucleotides. One or more of the odd numbered nucleotides of the first strand may be modified. One or more of the even numbered nucleotides of the first strand may be modified by at least a second modification, wherein the at least second modification is different from the modification on the one or more add nucleotides. At least one of the one or more modified even numbered nucleotides may be adjacent to at least one of the one or more modified odd numbered nucleotides.

A plurality of odd numbered nucleotides in the first strand may be modified in the nucleic acid of the invention. A plurality of even numbered nucleotides in the first strand may be modified by a second modification. The first strand may comprise adjacent nucleotides that are modified by a common modification. The first strand may also comprise adjacent nucleotides that are modified by a second different modification.

One or more of the odd numbered nucleotides of the second strand may be modified by a modification that is different to the modification of the odd numbered nucleotides on the first strand and/or one or more of the even numbered nucleotides of the second strand may be by the same modification of the odd numbered nucleotides of the first strand. At least one of the one or more modified even numbered nucleotides of the second strand may be adjacent to the one or more modified odd numbered nucleotides. A plurality of odd numbered nucleotides of the second strand may be modified by a common modification and/or a plurality of even numbered nucleotides may be modified by the same modification that is present on the first stand odd numbered nucleotides. A plurality of odd numbered nucleotides on the second strand may be modified by a second modification, wherein the second modification is different from the modification of the first strand odd numbered nucleotides.

The second strand may comprise adjacent nucleotides that are modified by a common modification, which may be a second modification that is different from the modification of the odd numbered nucleotides of the first strand.

In the nucleic acid of the invention, each of the odd numbered nucleotides in the first strand and each of the even numbered nucleotides in the second strand may be modified with a common modification and, each of the even numbered nucleotides may be modified in the first strand with a second modification and each of the odd numbered nucleotides may be modified in the second strand with the second modification.

The nucleic acid of the invention may have the modified nucleotides of the first strand shifted by at least one nucleotide relative to the unmodified or differently modified nucleotides of the second strand.

One or more or each of the odd numbered nucleotides may be modified in the first strand and one or more or each of the even numbered nucleotides may be modified in the second strand. One or more or each of the alternating nucleotides on either or both strands may be modified by a second modification. One or more or each of the even numbered nucleotides may be modified in the first strand and one or more or each of the even numbered nucleotides may be modified in the second strand. One or more or each of the alternating nucleotides on either or both strands may be modified by a second modification. One or more or each of the odd numbered nucleotides may be modified in the first strand and one or more of the odd numbered nucleotides may be modified in the second strand by a common modification. One or more or each of the alternating nucleotides on either or both strands may be modified by a second modification. One or more or each of the even numbered nucleotides may be modified in the first strand and one or more or each of the odd numbered nucleotides may be modified in the second strand by a common modification. One or more or each of the alternating nucleotides on either or both strands may be modified by a second modification.

The nucleic acid of the invention may comprise single or double stranded constructs that comprise at least two regions of alternating modifications in one or both of the strands. These alternating regions can comprise up to about 12 nucleotides but preferably comprise from about 3 to about 10 nucleotides. The regions of alternating nucleotides may be located at the termini of one or both strands of the nucleic acid of the invention. The nucleic acid may comprise from 4 to about 10 nucleotides of alternating nucleotides at each termini (3′ and 5′) and these regions may be separated by from about 5 to about 12 contiguous unmodified or differently or commonly modified nucleotides.

The odd numbered nucleotides of the first strand may be modified and the even numbered nucleotides may be modified with a second modification. The second strand may comprise adjacent nucleotides that are modified with a common modification, which may be the same as the modification of the odd numbered nucleotides of the first strand. One or more nucleotides of second strand may also be modified with the second modification. One or more nucleotides with the second modification may be adjacent to each other and to nucleotides having a modification that is the same as the modification of the odd numbered nucleotides of the first strand. The first strand may also comprise phosphorothioate linkages between the two nucleotides at the 3′ end and at the 5′ end. The second strand may comprise a phosphorothioate linkage between the two nucleotides at 5′ end. The second strand may also be conjugated to a ligand at the 5′ end.

The nucleic acid of the invention may comprise a first strand comprising adjacent nucleotides that are modified with a common modification. One or more of such nucleotides may be adjacent to one or more nucleotides which may be modified with a second modification. One or more nucleotides with the second modification may be adjacent. The second strand may comprise adjacent nucleotides that are modified with a common modification, which may be the same as one of the modifications of one or more nucleotides of the first strand. One or more nucleotides of second strand may also be modified with the second modification. One or more nucleotides with the second modification may be adjacent. The first strand may also comprise phosphorothioate linkages between the two nucleotides at the 5′ end and at the 3′ end. The second strand may comprise a phosphorothioate linkage between the two nucleotides at 3′ end. The second strand may also be conjugated to a ligand at the 5′ end.

The nucleotides for the purposes of modification as described herein (unless otherwise indicated) are numbered from 5′ to 3′ on the first strand and 3′ and 5′ on the second strand. Nucleotides numbered 1, 3, 5, 7, 9, 11, 13, 15, 17, 19, 21, 23 and 25 may be modified by a modification on the first strand. The nucleotides numbered 2, 4, 6, 8, 10, 12, 14, 16, 18, 20, 22 and 24 may be modified by a second modification on the first strand. The nucleotides numbered 1, 3, 5, 7, 9, 11, 13, 15, 17, 19, 21, 23 may be modified by a modification on the second strand. The nucleotides numbered 2, 4, 6, 8, 10, 12, 14, 16, 18, 20, 22 and 24 may be modified by a second modification on the second strand. Nucleotides are numbered for the sake of the nucleic acid of the present invention from 5′ to 3′ on the first strand and 3′ and 5′ on the second strand, unless otherwise indicated.

The nucleotides numbered 2, 4, 6, 8, 10, 12, 14, 16, 18, 20, 22 and 24 may be modified on the first strand. The nucleotides numbered 1, 3, 5, 7, 9, 11, 13, 15, 17, 19, 21, 23 may be modified by a second modification on the first strand. The nucleotides numbered 1, 3, 5, 7, 9, 11, 13, 15, 17, 19, 21, 23 may be modified by a modification on the second strand. The nucleotides numbered 2, 4, 6, 8, 10, 12, 14, 16, 18, 20, 22 and 24 may be modified by a second modification on the second strand.

Clearly, if the first and/or the second strand are shorter or longer than 25 nucleotides in length, such as 19 nucleotides in length, there are no nucleotides numbered 20, 21, 22, 23, 24 and 25 to be modified. The skilled person understands the description above to apply to shorter or longer strands, accordingly.

One or more modified nucleotides on the first strand may be paired with modified nucleotides on the second strand having a common modification. One or more modified nucleotides on the first strand may be paired with modified nucleotides on the second strand having a different modification. One or more modified nucleotides on the first strand may be paired with unmodified nucleotides on the second strand. One or more modified nucleotides on the second strand may be paired with unmodified nucleotides on the first strand. In other words, the alternating nucleotides can be aligned on the two strands such as, for example, all the modifications in the alternating regions of the second strand are paired with identical modifications in the first strand or alternatively the modifications can be offset by one nucleotide with the common modifications in the alternating regions of one strand pairing with dissimilar modifications (i.e. a second or further modification) in the other strand. Another option is to have dissimilar modifications in each of the strands.

The modifications on the first strand may be shifted by one nucleotide relative to the modified nucleotides on the second strand, such that common modified nucleotides are not paired with each other.

Modification of the 3′ and/or 5′ Ends

As explained above, the ends of the RNA strands may be conjugated. Additionally or alternatively to the conjugation described above, the ends of the RNA strands may be modified as described below.

The RNA of the present invention may include nucleic acid molecules comprising one or more modified nucleotides, abasic nucleotides, acyclic or deoxyribonucleotide (2′ deoxy) at the terminal 5′- or 3′-end on either or both of the sense or antisense strands.

When the modification is a 2′ deoxy modification, only a small portion of the nucleotides may have this modification, for example less than 15%, less than 10% or less than 5%.

Terminal modifications can be added for a number of reasons, including to modulate activity or to modulate resistance to degradation.

In one embodiment, the 5′- and 3′-end nucleotides of both the sense and antisense strands are unmodified. In another embodiment, the 5′-end nucleotide of the antisense strand is modified as described above. In another embodiment, the 5′-end nucleotide of the sense strand is modified as described above. In another embodiment, the 3′-end nucleotide of the antisense strand is modified as described above. In another embodiment, the 3′-end nucleotide of the sense strand is modified as described above. In another embodiment, the 5′-end nucleotide of the antisense strand and the 5′-end nucleotide of the sense strand are modified as described above. In another embodiment, the 3′-end nucleotide of the antisense strand and the 3′-end nucleotide of the sense strand are modified as described above. In another embodiment, the 5′-end nucleotide of the antisense strand and the 3′-end nucleotide of the sense strand are modified as described above. In another embodiment, the 3′-end nucleotide of the antisense strand and the 5-end nucleotide of the sense strand are modified as described above. In another embodiment, the 3′-end nucleotide of the sense strand and the 5-end nucleotide of the sense strand are modified as described above. In another embodiment, the 3′-end nucleotide of the antisense strand and both the 5′- and 3′-end nucleotides of the sense strand are modified. Both the 5′- and 3′-end nucleotides of the antisense strand may be modified as described above. In another embodiment, both the 5′- and 3′-end nucleotides of the sense strand are modified as described above. Examples of different kinds of end modification(s) are presented in Table 1.

TABLE 1
Examples of end modifications

| | Antisense strand | Sense strand |
|---|---|---|
| 1.5′-end | free OH | free OH |
| 3′-end | free OH | free OH |
| 2.5′-end | free OH | free OH |
| 3′-end | end modification | end modification |
| 3.5′-end | free OH | free OH |
| 3′-end | free OH | end modification |
| 4.5′-end | free OH | free OH |
| 3′-end | end modification | free OH |
| 5.5′-end | free OH | end modification |
| 3′-end | free OH | free OH |
| 6.5′-end | free OH | end modification |
| 3′-end | end modification | free OH |
| 7.5′-end | free OH | end modification |
| 3′-end | free OH | end modification |
| 8.5′-end | free OH | end modification |
| 3′-end | end modification | end modification |
| 9.5′ end | end modification | free OH |
| 3′ end | free OH | free OH |
| 10.5′ end | end modification | end modification |
| 3′ end | free OH | free OH |
| 11.5′ end | end modification | free OH |
| 3′ end | free OH | end modification |

1. Blunt Ends and Overhangs

The double stranded RNAs may be blunt ended at one end or on both ends e.g. blunt on both ends. Alternatively, the double stranded RNAs have an overhang at one end and a blunt end at the other. Alternatively, the double stranded RNAs have an overhang at both ends.

An “overhang” as used herein has its normal and customary meaning in the art, i.e. a single stranded portion of a nucleic acid that extends beyond the terminal nucleotide of a complementary strand in a double strand nucleic acid. Stability of a nucleic acid of the invention may be increased by including particular bases in overhangs, or to include modified nucleotides, in single strand overhangs, e.g., in a 5′ or 3′ overhang, or in both. Purine nucleotides may be included in overhangs. All or some of the bases in a 3′ or 5′ overhang may be modified. Modifications can include the use of modifications at the 2′ OH group of the ribose sugar and modifications in the phosphate group, such as phosphorothioate modifications. Overhangs need not be homologous with the target sequence.

The term “blunt end” includes double stranded nucleic acid whereby both strands terminate at the same position, regardless of whether the terminal nucleotide(s) are base paired. The terminal nucleotide of a first strand and a second strand at a blunt end may be base paired. The terminal nucleotide of a first strand and a second strand at a blunt end may not be paired. The terminal two nucleotides of a first strand and a second strand at a blunt end may be base paired. The terminal two nucleotides of a first strand and a second strand at a blunt end may not be paired.

When the double stranded RNAs have an overhang, the double stranded RNAs may have overhangs of 1 or more nucleotides one or both strands at one or both ends. The overhangs may be 1, 2, 3, 4, 5, 6, 7, 8, 9, or 10 nucleotides in length.

The overhang may comprise at least one deoxyribonucleotides and/or TT dinucleotide.

The nucleic acid may be blunt ended at the end with the 5′ end of the first strand and the 3′ end of the second strand or at the 3′-end of the first strand and the 5′ end of the second strand.

The nucleic acid may comprise an overhang at a 3′ or 5′ end. The nucleic acid may have a 3′ overhang on the first strand. The nucleic acid may have a 3′ overhang on the second strand. The nucleic acid may have a 5′ overhang on the first strand. The nucleic acid may have a 5′ overhang on the second strand. The nucleic acid may have an overhang at both the 5′ end and 3′ end of the first strand. The nucleic acid may have an overhang at both the 5′ end and 3′ end of the second strand. The nucleic acid may have a 5′ overhang on the first strand and a 3′ overhang on the second strand. The nucleic acid may have a 3′ overhang on the first strand and a 5′ overhang on the second strand. The nucleic acid may have a 3′ overhang on the first strand and a 3′ overhang on the second strand. The nucleic acid may have a 5′ overhang on the first strand and a 5′ overhang on the second strand.

An overhang at the 3′-end or 5′-end of the second strand or the first strand may be selected from consisting of 1, 2, 3, 4 and 5 nucleotides in length. Optionally, an overhang may consist of 1 or 2 nucleotides, which may or may not be modified.

The nucleic acid may, at the end of the nucleic acid that comprises the 5′ end of the first strand: a) be blunt ended or b) have a 3′ overhang of at least one nucleotide.

The nucleic acid may have an overhang at one end and a blunt end at the other. The nucleic acid may have an overhang at both ends. The nucleic acid may be blunt ended at both ends. The nucleic acid may be blunt ended at the end with the 5′-end of the first strand and the 3′-end of the second strand or at the 3′-end of the first strand and the 5′-end of the second strand.

The nucleic acid may comprise an overhang at a 3′- or 5′-end. The nucleic acid may have a 3′-overhang on the first strand. The nucleic acid may have a 3′-overhang on the second strand. The nucleic acid may have a 5′-overhang on the first strand. The nucleic acid may have a 5′-overhang on the second strand. The nucleic acid may have an overhang at both the 5′-end and 3′-end of the first strand. The nucleic acid may have an overhang at both the 5′-end and 3′-end of the second strand. The nucleic acid may have a 5′ overhang on the first strand and a 3′ overhang on the second strand. The nucleic acid may have a 3′ overhang on the first strand and a 5′ overhang on the second strand. The nucleic acid may have a 3′ overhang on the first strand and a 3′ overhang on the second strand. The nucleic acid may have a 5′ overhang on the first strand and a 5′ overhang on the second strand.

An overhang at the 3′-end or 5′ end of the second strand or the first strand may be selected from consisting of 1, 2, 3, 4 and 5 nucleotides in length. Optionally, an overhang may consist of 1 or 2 nucleotides, which may or may not be modified.

2. Phosphorylation

The 5′-end nucleotide of the antisense strand may be phosphorylated. In another embodiment, the 5′-end nucleotide of the sense strand may be phosphorylated. In another embodiment, the 5′-end nucleotides of both the antisense strand and the sense strand are phosphorylated. In another embodiment, the 5′-end nucleotide of the antisense strand is phosphorylated and the 5′-end nucleotide of the sense strand has a free hydroxyl group (5-OH). In another embodiment, the 5′-end nucleotide of the antisense strand is phosphorylated and the 5′-end nucleotide of the sense strand is modified. In another embodiment the 5′-end nucleotide of the antisense strand carries a 5′-(E)-vinylphosphonate.

Nucleic acids of the invention, on the first or second strand, may be 5′ phosphorylated or include a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g., 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g., RP(OH)(O)—O-5′-, (OH)₂(O)P-5′-CH₂—), 5′vinylphosphonate, 5′-alkyletherphosphonates (R=alkylether=methoxymethyl (MeOCH₂—), ethoxymethyl, etc., e.g., RP(OH)(O)—O-5′-).

3. Capping with Small Chemical Groups

The nucleic acids according to the present invention may be modified by capping of the 3′ and/or 5′ ends of either strand with small chemical groups. Examples of modifications to end nucleotides include, but are not limited to, biotin, abasics, amino, fluoro, chloro, bromo, CN, CF, methoxy, imidazole, carboxylate, thioate, C₁ to C₁₀ alkyl (e.g. C₁ to C alkyl, e.g. methyl, ethyl, propyl), substituted lower alkyl, alkaryl or arylalkyl, OCF₃, OCN, O—, S—, or N-alkyl; O—, S—, or N-alkenyl; SO—CH₃; SO₂CH₃; ONO₂; NO₂, N₃; heterocycloalkyl; heterocycloalkaryl; aminoalkylamino; polyalkylamino or substituted silyl, as, among others, described, e.g., in WO 99/54459, EP 0 586 520 B1 or EP 0 618 925 B1, incorporated by reference in their entireties.

In another aspect, the 5′-end of the antisense strand, the 5′-end of the sense strand, the 3′-end of the antisense strand or the 3′-end of the sense strand may be covalently connected to a prodrug moiety. In one embodiment, the moiety may be cleaved in an endosome. In another the moiety may be cleaved in the cytoplasm.

4. Modifications to Phosphate Backbone

Another aspect relates to modifications to a phosphate backbone. All or a portion of the nucleotides of the RNA of the invention may be linked through phosphodiester bonds, as found in unmodified nucleic acid. A RNA of the present invention however, may comprise a modified phosphodiester linkage. The phosphodiester linkages of either the antisense stand or the sense strand may be modified to independently include at least one heteroatom selected from nitrogen and sulfur. In one embodiment, a phosphodiester group connecting a ribonucleotide to an adjacent ribonucleotide is replaced by a modified group.

In one embodiment, all of the nucleotides of the antisense strand are linked through phosphodiester bonds. In another embodiment, all of the nucleotides of the antisense duplex region are linked through phosphodiester bonds. In another embodiment, all of the nucleotides of the sense strand are linked through phosphodiester bonds. In another embodiment, all of the nucleotides of the sense duplex region are linked through phosphodiester bonds. In another embodiment, the antisense strand comprises a number of modified phosphodiester groups selected from 1, 2, 3, 4, 5, 6, 7, 8, 9 or 10. In another embodiment, the antisense duplex region comprises a number of modified phosphodiester groups selected from 1, 2, 3, 4, 5, 6, 7, 8, 9 or 10. In another embodiment, the sense strand comprises a number of modified phosphodiester groups selected from 1, 2, 3, 4, 5, 6, 7, 8, 9 or 10. In another embodiment, the sense duplex region comprises a number of modified phosphodiester groups selected from 1, 2, 3, 4, 5, 6, 7, 8, 9 or 10.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulphur. One, each or both non-linking oxygens in the phosphate group can be independently any one of S, Se, B, C, H, N, or OR (R is alkyl or aryl).

The phosphate linker can also be modified by replacement of a linking oxygen with nitrogen (bridged phosphoroamidates), sulfur (bridged phosphorothioates) and carbon (bridged methylenephosphonates). The replacement can occur at a terminal oxygen. Replacement of the non-linking oxygens with nitrogen is possible.

The phosphate linker and ribose sugar may be replaced by nuclease resistant nucleotides.

Examples include the mophilino, cyclobutyl, pyrrolidine and peptide nucleic acid (PNA) nucleoside surrogates. In certain embodiments, PNA surrogates may be used.

5. Phosphorothioate Internucleotide Linkages

The nucleic acids according to the present invention may comprise one or more phosphorothioate internucleotide linkage on one or more of the terminal ends of the first and/or the second strand. By phosphorothioate internucleotide linkages it is meant that the linkage between the nucleotide and the adjacent nucleotide comprises a phosphorothioate group instead of a standard phosphate group. Preferably the phosphorothioate internucleotide linkages may be distributed across the entire nucleotide sequences and may occur in any number at any position. Preferably the nucleic acids can comprise between one to ten phosphorothioate internucleotide linkages. Preferably the first RNA strand (i.e. the antisense strand) has one to six phosphorothioate internucleotide linkages at each end. Preferably the second RNA strand (i.e. the sense strand) has one to six phosphorothioate internucleotide linkages at each end. Preferably the antisense strand has at least 1 phosphorothioate modification at each end. Preferably the antisense strand has 1-3 phosphorothioate modifications at each end. Most preferably the antisense strand has 2 phosphorothioate modifications at each end. Preferably the sense strand has at least 1 phosphorothioate modification at the 3′ end. Preferably the sense strand has 1-3 phosphorothioate modifications at the 3′ end. Most preferably the sense strand has 2 phosphorothioate modifications at the 3′ end.

6. Phosphorodithioate Internucleotide Linkages

The nucleic acids according to the present invention may comprise one or more phosphorodithioate internucleotide linkage on one or more of the terminal ends of the first and/or the second strand. By phosphorodithioate internucleotide linkages it is meant that the linkage between the nucleotide and the adjacent nucleotide comprises a phosphorodithioate group instead of a standard phosphate group. Preferably the phosphorodithioate internucleotide linkages may be distributed across the entire nucleotide sequences and may occur in any number at any position. Preferably the nucleic acids can comprise between one to ten phosphorodithioate internucleotide linkages. Preferably the first RNA strand (i.e. the antisense strand) has one to six phosphorodithioate internucleotide linkages at each end. Preferably the second RNA strand (i.e. the sense strand) has one to six phosphorodithioate internucleotide linkages at each end. Preferably the antisense strand has at least 1 phosphorodithioate modification at the 3′ end. Preferably the antisense strand has 1-3 phosphorodithioate modifications at the 3′ end. Most preferably the antisense strand has 1 phosphorodithioate modifications at the 3′end. Preferably the sense strand has 1-3 phosphorodithioate modification at both ends. Preferably the sense strand has at least 1 phosphorodithioate modification at both ends. Preferably the sense strand has at least 1 phosphorodithioate modification at the 3′ end. Preferably the sense strand has 1-3 phosphorodithioate modifications at the 3′ end. Most preferably the sense strand has 1 phosphorodithioate modification at the 3′ end.

In further embodiments of the invention, the invention relates to any nucleic acid, conjugated nucleic acid, nucleic acid for use, method, composition or use according to any disclosure herein, wherein the nucleic acid comprises a phosphorodithioate linkage, optionally wherein the linkage is between the 2 most 5′ nucleosides and/or the 2 most 3′ nucleosides of the second strand, and/or optionally wherein the nucleic acid additionally does not comprise any internal phosphorothioate linkages.

7. Labelling and Protecting Groups

The 3′ and 5′ ends of an oligonucleotide can be conjugated to other functional molecular entities such as labelling moieties, e.g., fluorophores (e.g., pyrene, TAMRA, fluorescein, an Alexa dye, Cy3 or Cy5 dyes) or protecting groups (based e.g., on sulfur, silicon, boron or ester).

The functional molecular entities can be attached to the sugar through a phosphate group and/or a linker. The terminal atom of the linker can connect to or replace the linking atom of the phosphate group or the C-3′ or C-5′O, N, S or C group of the sugar. Alternatively, the linker can connect to or replace the terminal atom of a nucleotide surrogate (e.g., PNAs). These spacers or linkers can include e.g., —(CH2)n-, —(CH2)nN-, —(CH2)nO-, —(CH2)nS-, O(CH2CH2O)nCH2CH2OH (e.g., n=3 or 6), abasic sugars, amide, carboxy, amine, oxyamine, oxyimine, thioether, disulfide, thiourea, sulfonamide, or morpholino, or biotin and fluorescein reagents. The 3′end can be an —OH group.

8. Miscellaneous End Modifications

Terminal modifications can also be useful for enhancing uptake. Useful modifications for this include modifying the terminal ends of either strand with cholesterol. Terminal modifications can also be useful for cross-linking an RNA agent to another moiety.

Other examples of terminal modifications include dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic carriers (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles).

Certain moieties may be linked to the 5′ terminus of the first strand or the second strand and includes abasic ribose moiety, abasic deoxyribose moiety, modifications abasic ribose and abasic deoxyribose moieties including 2′ O alkyl modifications; inverted abasic ribose and abasic deoxyribose moieties and modifications thereof, C6-imino-Pi; a mirror nucleotide including L-DNA and L-RNA; 5′OMe nucleotide; and nucleotide analogs including 4′,5′-methylene nucleotide; 1-(beta-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 12-aminododecyl phosphate; hydroxypropyl phosphate; 1,5-anhydrohexitol nucleotide; alpha-nucleotide; threo-pentofuranosyl nucleotide; acyclic 3′,4′-seco nucleotide; 3,4-dihydroxybutyl nucleotide; 3,5-dihydroxypentyl nucleotide, 5′-5′-inverted abasic moiety; 1,4-butanediol phosphate; 5′-amino; and bridging or non bridging methylphosphonate and 5′-mercapto moieties.

9. Modifications to the Base

One or more nucleotides of a RNA of the present invention may comprise a modified base. Adenine, guanine, cytosine and uracil are the most common bases found in RNA. These bases can be modified or replaced to provide RNAs having improved properties. E.g., nuclease resistant oligoribonucleotides can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases and “universal bases” can be employed.

In one aspect, the RNA comprises at least one nucleotide comprising a modified base. In one embodiment, the modified base is on the antisense strand. In another embodiment, the modified base is on the sense strand. In another embodiment, the modified base is in the duplex region. In another embodiment, the modified base is outside the duplex region, i.e., in a single stranded region. In another embodiment, the modified base is on the antisense strand and is outside the duplex region. In another embodiment, the modified base is on the sense strand and is outside the duplex region. In another embodiment, the 3′-terminal nucleotide of the antisense strand is a nucleotide with a modified base. In another embodiment, the 3′-terminal nucleotide of the sense strand is nucleotide with a modified base. In another embodiment, the 5′-terminal nucleotide of the antisense strand is nucleotide with a modified base. In another embodiment, the 5′-terminal nucleotide of the sense strand is nucleotide with a modified base.

In one embodiment, a RNA may have 1 modified base. In another embodiment, a RNA may have about 2-4 modified bases. In another embodiment, a RNA has about 4-6 modified bases. In another embodiment, a RNA has about 6-8 modified bases. In another embodiment, a RNA has about 8-10 modified bases. In another embodiment, a RNA has about 10-12 modified bases. In another embodiment, a RNA has about 12-14 modified bases. In another embodiment, a RNA has about 14-16 modified bases. In another embodiment, a RNA has about 16-18 modified bases. In another embodiment, a RNA has about 18-20 modified bases. In another embodiment, a RNA has about 20-22 modified bases. In another embodiment, a RNA has about 22-24 modified bases. In another embodiment, a RNA has about 24-26 modified bases. In another embodiment, a RNA has about 26-28 modified bases. In each case the RNA comprising said modified bases retains at least 50% of its activity as compared to the same RNA but without said modified bases.

Examples of modified bases include 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and 0-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6,N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3-carboxypropyl)uracil, 3-methylcytosine, 5-methylcytosine, N4-acetyl cytosine, 2-thiocytosine, N6-methyladenine, N6-isopentyladenine, 2-methylthio-N6-isopentenyladenine, N-methylguanines, or O-alkylated bases.

The modified base may be a modified purine or a pyrimidine. In another embodiment, at least half of the purines are modified. In another embodiment, at least half of the pyrimidines are modified. In another embodiment, all of the purines are modified. In another embodiment, all of the pyrimidines are modified. In another embodiment, the RNA may comprise a nucleotide comprising a modified base as described above.

In another aspect, a RNA of the present invention comprises an abasic nucleotide. The term “abasic” as used herein, refers to moieties lacking a base or having other chemical groups in place of a base at the 1′ position, for example a 3′,3′-linked or 5′,5′-linked deoxyabasic ribose derivative. As used herein, a nucleotide with a modified base does not include abasic nucleotides.

In one aspect, the RNA comprises at least one abasic nucleotide. In one embodiment, the abasic nucleotide is on the antisense strand. In another embodiment, the abasic nucleotide is on the sense strand. In another embodiment, the abasic nucleotide is in the duplex region. In another embodiment, the abasic nucleotide is outside the duplex region. In another embodiment, the abasic nucleotide is on the antisense strand and is outside the duplex region. In another embodiment, the abasic nucleotide is on the sense strand and is outside the duplex region. In another embodiment, the 3′-terminal nucleotide of the antisense strand is an abasic nucleotide. In another embodiment, the 3′-terminal nucleotide of the sense strand is an abasic nucleotide. In another embodiment, the 5′-terminal nucleotide of the antisense strand is an abasic nucleotide. In another embodiment, the 5′-terminal nucleotide of the sense strand is an abasic nucleotide. In another embodiment, a RNA has a number of abasic nucleotides selected from 1, 2, 3, 4, 5 and 6.

10. Modifications to Sugar Moiety

Another aspect relates to modifications of a sugar moiety. One or more nucleotides of a RNA of the present invention may comprise a modified ribose moiety. Modifications at the 2′-position where the 2′-OH is substituted include the non-limiting examples selected from alkyl, substituted alkyl, alkaryl-, arylalkyl-, —F, —Cl, —Br, —CN, —CF3, —OCF3, —OCN, —O-alkyl, —S-alkyl, HS-alkyl-O, —O— alkenyl, —S-alkenyl, —N-alkenyl, —SO-alkyl, -alkyl-OSH, -alkyl-OH, —O-alkyl-OH, —O-alkyl-SH, —S— alkyl-OH, —S-alkyl-SH, -alkyl-S-alkyl, -alkyl-O-alkyl, —ONO2, —NO2, —N3, —NH2, alkylamino, dialkylamino-, aminoalkyl-, aminoalkoxy, aminoacid, aminoacyl-, —ONH2, —O-aminoalkyl, —O-aminoacid, —O-aminoacyl, heterocycloalkyl-, heterocycloalkaryl-, aminoalkylamino-, polyalklylamino-, substituted silyl-, methoxyethyl- (MOE), alkenyl and alkynyl. “Locked” nucleic acids (LNA) in which the 2′ hydroxyl is connected, e.g., by a methylene bridge, to the 4′ carbon of the same ribose sugar is further included as a 2′ modification of the present invention. Preferred substituents are 2′-methoxyethyl, 2′-O—CH₃ (2′-OMe), 2′-O-allyl, 2′-C-allyl, and 2′-fluoro (2′-F).

The modification and/or modifications may each and individually be selected from the group consisting of 3′ terminal deoxy thymine, 2′-OMe, a 2′ deoxy modification, a 2′ amino modification, a 2′ alkyl modification, a morpholino modification, a phosphoramidate modification, 5′-phosphorothioate group modification, a 5′ phosphate or 5′ phosphate mimic modification and a cholesteryl derivative or a dodecanoic acid bisdecylamide group modification and/or the modified nucleotide may be any one of a locked nucleotide, an abasic nucleotide (which lack a nucleobase at C-1′) or a non natural base comprising nucleotide. At least one modification may be 2′-O-methyl (2′-OMe) and/or at least one modification may be 2′-F.

When the modification is a 2′ deoxy modification, only a small portion of the nucleotides may have this modification, for example less than 15%, less than 10% or less than 5%.

In one embodiment, the RNA comprises 1-5 2′-modified nucleotides. In another embodiment, the RNA comprises 5-10 2′-modified nucleotides. In another embodiment, the RNA comprises 15-20 2′-modified nucleotides. In another embodiment, the RNA comprises 20-25 2′-modified nucleotides. In another embodiment, the RNA comprises 25-30 2′-modified nucleotides. In an embodiment, the sugar group can also contain one or more carbons that possess the opposite stereochemical configuration than that of the corresponding carbon in ribose. Thus, a modified nucleotides may contain a sugar such as arabinose.

In one embodiment, the RNA comprises 1-5 2′-OMe modified nucleotides. In another embodiment, the RNA comprises 5-10 2′-OMe modified nucleotides. In another embodiment, the RNA comprises 15-20 2′-OMe modified nucleotides. In another embodiment, the RNA comprises 20-25 2′-OMe modified nucleotides. In another embodiment, the RNA comprises 25-30 2′-OMe modified nucleotides.

In one embodiment, the RNA duplex region comprises 1-5 2′-OMe modified nucleotides. In another embodiment, the RNA duplex region comprises 5-10 2′-OMe modified nucleotides. In another embodiment, the RNA duplex region comprises 15-20 2′-OMe modified nucleotides. In another embodiment, the RNA duplex region comprises 20-25 2′-OMe modified nucleotides. In another embodiment, the RNA duplex region comprises 25-30 2′-OMe modified nucleotides.

In one embodiment, the RNA comprises an antisense strand of 19 nucleotides in length and a sense strand 19 nucleotides in length. In another embodiment, the RNA comprises an antisense strand 20 nucleotides in length and a sense strand 20 nucleotides in length. In another embodiment, the RNA comprises an antisense strand 21 nucleotides in length and a sense strand 21 nucleotides in length. In another embodiment, the RNA comprises an antisense strand 22 nucleotides in length and a sense strand 22 nucleotides in length. In another embodiment, the RNA comprises an antisense strand 23 nucleotides in length and a sense strand 23 nucleotides in length.

In any of the above embodiments, the antisense strand comprises 2′-OMe modifications at nucleotides 1, 3, 5, 7, 9, 11, 13, 15, 17, 19, 21 and 23, and the sense strand comprises 2′-OMe modifications at nucleotides 2, 4, 6, 8, 10, 12, 14, 16, 18, 20 and 22 wherein said antisense strand is numbered from 5′-3′ and said sense strand is numbered from 3′-5′.

In another embodiment, the RNA comprises an antisense strand 18-23 nucleotides in length and a sense strand 18-23 nucleotides in length, wherein said antisense strand comprises 2′-OMe modifications at nucleotides 3, 5, 7, 9, 11, 13, 15 and 17 (such as 5, 7, 9, 11, 13 and 15, e.g. 7, 9, 11, 13), and wherein said sense strand comprises 2′-OMe modifications at nucleotides 4, 6, 8, 10, 12, 14 and 16 (such as 6, 8, 10, 12 and 14 e.g. 8, 10 and 12), wherein said antisense strand is numbered from 5′-3′ and said sense strand is numbered from 3′-5′.

In another embodiment, the RNA comprises an antisense strand 18-23 nucleotides in length and a sense strand 18-23 nucleotides in length, wherein said antisense strand comprises 2′-OMe modifications at nucleotides 7, 9 and 11, and wherein said sense strand comprises 2′-OMe modifications at nucleotides 8, 10 and 12, wherein said antisense strand is numbered from 5′-3′ and said sense strand is numbered from 3′-5′.

In another embodiment, the RNA comprises an antisense strand 18-23 nucleotides in length and a sense strand 18-23 nucleotides in length, wherein said antisense strand comprises 2′-OMe modifications at nucleotides 7 and 9, and wherein said sense strand comprises 2′-OMe modifications at nucleotides 8 and 10, wherein said antisense strand is numbered from 5′-3′ and said sense strand is numbered from 3′-5′.

In another embodiment, the RNA comprises an antisense strand 18-23 nucleotides in length and a sense strand 18-23 nucleotides in length, wherein said antisense strand comprises 2′-OMe modifications at nucleotides 9 and 11, and wherein said sense strand comprises 2′-OMe modifications at nucleotides 8 and 10, wherein said antisense strand is numbered from 5′-3′ and said sense strand is numbered from 3′-5′.

One aspect is a nucleic acid for inhibiting expression of a target gene in a cell, comprising at least one duplex region that comprises at least a portion of a first strand and at least a portion of a second strand that is at least partially complementary to the first strand, wherein said first strand is at least partially complementary to at least a portion of RNA transcribed from said target gene, wherein said first strand includes modified nucleotides or unmodified nucleotides at a plurality of positions in order to facilitate processing of the nucleic acid by RISC.

In one aspect “facilitate processing by RISC” means that the nucleic acid can be processed by RISC, for example any modification present will permit the nucleic acid to be processed by RISC, suitably such that SiRNA activity can take place.

A nucleic acid as disclosed herein, wherein the nucleotides at positions 2 and 14 from the 5′ end of the first strand are not modified with a 2′-OMe modification, and the nucleotide on the second strand which corresponds to position 13 of the first strand is not modified with a 2′-OMe modification.

A nucleotide on the second strand that “corresponds to” a position on the first strand is suitably the nucleotide that base pairs with that nucleotide on the first strand.

In one aspect the nucleotide on the second strand which corresponds to position 13 of the first strand is the nucleotide that forms a base pair with position 13 of the first strand. In one aspect the nucleotide on the second strand which corresponds to position 11 of the first strand is the nucleotide that forms a base pair with position 11 of the first strand. In one aspect the nucleotide on the second strand which corresponds to position 12 of the first strand is the nucleotide that forms a base pair with position 12 of the first strand. This nomenclature may be applied to other positions of the second strand.

For example, in a 19-mer nucleic acid which is double stranded and blunt ended, position 13 of the first strand would pair with position 7 of the second strand. Position 11 of the first strand would pair with position 9 of the second strand. This nomenclature may be applied to other positions of the second strand.

The nucleotide that corresponds to position 13 of the first strand is suitably position 13 of the second strand, counting from the 3′ of the second strand, starting from the first nucleotide of the double stranded region. Likewise position 11 of the second strand is suitably the 11th nucleotide from the 3′ of the second strand, starting from the first nucleotide of the double stranded region. This nomenclature may be applied to other positions of the second strand.

In one aspect, in the case of a partially complementary first and second strand, the nucleotide on the second strand that “corresponds to” a position on the first strand may not necessarily form a base pair if that position is the position in which there is a mismatch, but the principle of the nomenclature still applies.

Preferred is a first and second strand that are fully complementary over the duplex region (ignoring any overhang regions) and there are no mismatches within the double stranded region of the nucleic acid.

Also preferred are:

A nucleic acid as disclosed herein, wherein the nucleotides at positions 2 and 14 from the 5′ end of the first strand are not modified with a 2′-OMe modification, and the nucleotide on the second strand which corresponds to position 11 of the first strand is not modified with a 2′-OMe modification.

A nucleic acid as disclosed herein, wherein the nucleotides at positions 2 and 14 from the 5′ end of the first strand are not modified with a 2′-OMe modification, and the nucleotides on the second strand which corresponds to position 11 and 13 of the first strand are not modified with a 2′-OMe modification.

In one aspect the nucleotide on the second strand which corresponds to position 12 of the first strand is not modified with a 2′-OMe modification. This limitation on the nucleic acid may be seen with any other limitation described herein.

Therefore another aspect of the invention is a nucleic acid as disclosed herein, wherein the nucleotides at positions 2 and 14 from the 5′ end of the first strand are not modified with a 2′-OMe modification, and the nucleotides on the second strand which corresponds to position 11-13 of the first strand are not modified with a 2′-OMe modification.

A nucleic acid as disclosed herein, wherein the nucleotides at positions 2 and 14 from the 5′ end of the first strand are not modified with a 2′-OMe modification, and the nucleotides on the second strand which correspond to position 11, or 13, or 11 and 13, or 11-13 of the first strand are modified with a 2′-F modification.

A nucleic acid as disclosed herein, wherein the nucleotides at positions 2 and 14 from the 5′ end of the first strand are modified with a 2′-F modification, and the nucleotides on the second strand which correspond to position 11, or 13, or 11 and 13, or 11-13 of the first strand are not modified with a 2′-OMe modification A nucleic acid as disclosed herein, wherein the nucleotides at positions 2 and 14 from the 5′ end of the first strand are modified with a 2′-F modification, and the nucleotides on the second strand which correspond to position 11, or 13, or 11 and 13, or 11-13 of the first strand are modified with a 2′-F modification.

A nucleic acid as disclosed herein wherein greater than 50% of the nucleotides of the first and/or second strand comprise a 2′-OMe modification, such as greater than 55%, 60%, 65%, 70%, 75%, 80%, or 85%, or more, of the first and/or second strand comprise a 2′-OMe modification, preferably measured as a percentage of the total nucleotides of both the first and second strands.

A nucleic acid as disclosed herein wherein greater than 50% of the nucleotides of the first and/or second strand comprise a naturally occurring RNA modification, such as wherein greater than 55%, 60%, 65%, 70%, 75%, 80%, or 85% or more of the first and/or second strands comprise such a modification, preferably measured as a percentage of the total nucleotides of both the first and second strands. Suitable naturally occurring modifications include, as well as 2′-OMe, other 2′ sugar modifications, in particular a 2′ H modification resulting in a DNA nucleotide.

A nucleic acid as disclosed herein comprising no more than 20%, such as no more than 15% such as more than 10%, of nucleotides which have 2′ modifications that are not 2′-OMe modifications on the first and/or second strand, preferably as a percentage of the total nucleotides of both the first and second strands.

A nucleic acid as disclosed herein comprising no more than 20%, (such as no more than 15% or no more than 10%) of 2′-F modifications on the first and/or second strand, preferably as a percentage of the total nucleotides of both strands.

A nucleic acid as disclosed herein, wherein all nucleotides are modified with a 2′-OMe modification except positions 2 and 14 from the 5′ end of the first strand and the nucleotides on the second strand which correspond to position 11, or 13, or 11 and 13, or 11-13 of the first strand. Preferably the nucleotides that are not modified with 2′-OMe are modified with fluoro at the 2′ position.

Preferred is a nucleic acid as disclosed herein wherein all nucleotides of the nucleic acid are modified at the 2′ position of the sugar. Preferably these nucleotides are modified with a 2′-F modification where the modification is not a 2′-OMe modification.

Nucleic acids of the invention may comprise one or more nucleotides modified at the 2′ position with a 2′ H, and therefore having a DNA nucleotide within the nucleic acid. Nucleic acids of the invention may comprise DNA nucleotides at positions 2 and/or 14 of the first strand counting from the 5′ end of the first strand. Nucleic acids may comprise DNA nucleotides on the second strand which correspond to position 11, or 13, or 11 and 13, or 11-13 of the first strand.

In one aspect there is no more than one DNA per nucleic acid of the invention.

Nucleic acids of the invention may comprise one or more LNA nucleotides. Nucleic acids of the invention may comprise LNA nucleotides at positions 2 and/or 14 of the first strand counting from the 5′ end of the first strand. Nucleic acids may comprise LNA on the second strand which correspond to position 11, or 13, or 11 and 13, or 11-13 of the first strand.

Preferably the nucleic acid as disclosed herein is an siRNA.

In one aspect the nucleic acid is modified on the first strand with alternating 2′-OMe modifications and 2′-F modifications, and positions 2 and 14 (starting from the 5′ end) are modified with 2′-F.

Preferably the second strand is modified with 2′-F modifications at nucleotides on the second strand which correspond to position 11, or 13, or 11 and 13, or 11-13 of the first strand. Preferably the second strand is modified with 2′-F modifications at positions 11-13 counting from the 3′ end starting at the first position of the complementary (double stranded) region, and the remaining modifications are naturally occurring modifications, preferably 2′-OMe.

All the features of the nucleic acids can be combined with all other aspects of the invention disclosed herein.

In particular, preferred are nucleic acids which are siRNA molecules wherein the nucleotides at positions 2 and 14 from the 5′ end of the first strand are not modified with a 2′-OMe modification, and the nucleic acid comprises one or more or all of:
(i) an inverted nucleotide, preferably a 3′-3′ linkage at the 3′ end of the second strand;
(ii) one or more phosphorodithioate linkages;
(iii) the second strand nucleotide corresponding to position 11 or 13 of the first strand is not modified with a 2′-OMe modification, preferably wherein one or both of these positions comprise a 2′-F modification
(iv) the nucleic acid comprises at least 80% of all nucleotides having a 2′-OMe modification
(v) the nucleic acid comprises no more than 20% of nucleotides which have 2′-F modifications.

Also provided by the present invention is a nucleic acid as disclosed herein, wherein the nucleotides at positions 2 and 14 from the 5′ end of the first strand and the nucleotides at positions 7 and/or 9, or 7-9 from the 5′ end of the second strand are modified with a 2′-F modification, and at least 90% of the remaining nucleotides are 2′-OMe modified or comprise another naturally occurring 2′ modification.

Specific preferred examples, for a blunt double stranded 19 base nucleic acid, with no overhang, are:

A nucleic acid as disclosed herein, wherein the nucleotides at positions 2 and 14 from the 5′ end of the first strand are not modified with a 2′-OMe modification, and the nucleotide at position 7 from the 5′ end of the second strand is not modified with a 2′-OMe modification.

A nucleic acid as disclosed herein, wherein the nucleotides at positions 2 and 14 from the 5′ end of the first strand are not modified with a 2′-OMe modification, and the nucleotide at position 9 from the 5′ end of the second strand is not modified with a 2′-OMe modification A nucleic acid as disclosed herein, wherein the nucleotides at positions 2 and 14 from the 5′ end of the first strand are not modified with a 2′-OMe modification, and the nucleotides at position 7 and 9 from the 5′ end of the second strand are not modified with a 2′-OMe modification.

A nucleic acid as disclosed herein, wherein the nucleotides at positions 2 and 14 from the 5′ end of the first strand are not modified with a 2′-OMe modification, and the nucleotides at positions 7-9 from the 5′ end of the second strand are not modified with a 2′-OMe modification.

A nucleic acid as disclosed herein, wherein the nucleotides at positions 2 and 14 from the 5′ end of the first strand are not modified with a 2′-OMe modification, and the nucleotides at positions 7 and/or 9, or 7-9 from the 5′ end of the second strand are modified with a 2′-F modification.

A nucleic acid as disclosed herein, wherein the nucleotides at positions 2 and 14 from the 5′ end of the first strand are modified with a 2′-F modification, and the nucleotides at positions 7 and/or 9, or 7-9 from the 5′ end of the second strand are not modified with a 2′-OMe modification A nucleic acid as disclosed herein, wherein the nucleotides at positions 2 and 14 from the 5′ end of the first strand are modified with a 2′-F modification, and the nucleotides at positions 7 and/or 9, or 7-9 from the 5′ end of the second strand are modified with a 2′-F modification.

For a nucleic acid comprising a 20 base pair duplex region, the second strand preferably does not have a 2′-OMe group at nucleotides 8 or 9 or 10 counting from the 5′ end of the duplex corresponding to positions 13, 12, and 11 of the first strand respectively.

For a nucleic acid comprising a 21 base pair duplex region, the second strand preferably does not have a 2′-OMe group at nucleotides 9 or 10 or 11 counting from the 5′ end of the duplex corresponding to positions 13, 12, and 11 of the first strand respectively.

In the preceding disclosure, a 2′-NH₂ modification may be used as an alternative to a 2′-F modification in any aspect of the invention, especially in siRNA modification. A 2′-F modification is however more preferred.

In any aspect or embodiment of the invention described herein, the nucleic acid (or use, method, composition or any other teaching involving a nucleic acid) comprises one DNA nucleotide at position 2, or 14, counting from the 5′ end of the first strand and additionally, and/or alternatively, comprises 1, 2, or 3 DNA nucleotides at positions on the second strand which correspond to any one, two or three positions 11, 12 and 13 of the first strand.

In any aspect or embodiment of the invention described herein, the nucleic acid (or use, method, composition or any other teaching involving a nucleic acid) comprises a DNA nucleotide, or a 2′-F modification, at a position or positions on the second strand which corresponds to positions 11-13 of the first strand. More than one modification may be present.

In any aspect or embodiment of the invention described herein, the nucleic acid—or any use, method, composition or any other teaching involving a nucleic acid herein—does not comprise a bulky modification group—such as a 2′-OMe group—at any one of position 2, or 14, or both, counting from the 5′ end of the first strand, and/or at any position of the second strand which corresponds to positions 11, 12 or 13 of the first strand. A bulky modification may be any modification that is bigger than an ′OH group, for example, at the 2′ position of the RNA sugar moiety.

The modification and/or modifications may each and individually be selected from the group consisting of 3′-terminal deoxy-thymine, 2′-O-methyl (2′-OMe), a 2′-deoxy-modification, a 2′-amino-modification, a 2′-alkyl-modification, a morpholino modification, a phosphoramidate modification, 5′-phosphorothioate group modification, a 5′ phosphate or 5′ phosphate mimic modification and a cholesteryl derivative or a dodecanoic acid bisdecylamide group modification and/or the modified nucleotide may be any one of a locked nucleotide, an abasic nucleotide or a non-natural base comprising nucleotide.

At least one modification may be 2′-OMe and/or at least one modification may be 2′-F. Further modifications as described herein may be present on the first and/or second strand.

Throughout the description of the invention, “same or common modification” means the same modification to any nucleotide, be that A, G, C or U modified with a group such as such as a methyl group or a fluoro group. Is it not taken to mean the same addition on the same nucleotide. For example, 2′-F-dU, 2′-F-dA, 2′-F-dC, 2′-F-dG are all considered to be the same or common modification, as are 2′-OMe-rU, 2′-OMe-rA; 2′-OMe-rC; 2′-OMe-rG. A 2′-F modification is a different modification to a 2′-OMe modification.

Some representative modified nucleic acid sequences of the present invention are shown in the examples. These examples are meant to be representative and not limiting.

Preferably, the nucleic acid may comprise a modification and the second or further modification which are each and individually selected from the group comprising 2′-OMe modification and 2′-F modification. The nucleic acid may comprise a modification that is 2′-OMe that may be a first modification, and a second modification that is 2′-F. The nucleic acid of the invention may also include a phosphorothioate modification and/or a deoxy modification which may be present in or between the terminal 1, 2 or 3 nucleotides of each or any end of each or both strands.

The nucleic acid of the conjugate may have any of the following preferred features:
(i) the nucleotides at positions 2 and 14 from the 5′ end of the first strand are modified;
(ii) the nucleotides at positions 2 and 14 from the 5′end of the first strand are not modified with a 2′-OMe modification, and the nucleotide on the second strand which corresponds to position 13 of the first strand is not modified with a 2′-OMe modification;
(iii) the nucleotides at positions 2 and 14 from the 5′end of the first strand are not modified with a 2′-OMe modification, and the nucleotide on the second strand which corresponds to position 11 of the first strand is not modified with a 2′-OMe modification;
(iv) nucleotides at positions 2 and 14 from the 5′ end of the first strand are not modified with a 2′-OMe modification, and the nucleotides on the second strand which corresponds to position 11 and 13 of the first strand are not modified with a 2′-OMe modification;
(v) the nucleotides on the second strand corresponding to positions 11 and/or 13 from the 5′ end of the first strand are modified;
(vi) the nucleotides at positions 2 and 14 from the 5′end of the first strand are not modified with a 2′-OMe modification, and the nucleotides on the second strand which correspond to position 11, or 13, or 11 and 13, or 11-13 of the first strand are modified with a 2′-F modification;
(vii) the nucleotides at positions 2 and 14 from the 5′ end of the first strand are modified with a 2′-F modification, and the nucleotides on the second strand which correspond to position 11, or 13, or 11 and 13, or 11-13 of the first strand are not modified with a 2′-OMe modification;
(viii) the nucleotides at positions 2 and 14 from the 5′ end of the first strand are modified with a 2′-F modification, and the nucleotides on the second strand which correspond to position 11, or 13, or 11 and 13, or 11-13 of the first strand are modified with a 2′-F modification;
(ix) greater than 50% of the nucleotides of the first and/or second strand comprise a 2′-OMe modification, such as greater than 55%, 60%, 65%, 70%, 75%, 80%, or 85%, or more, of the first and/or second strand comprise a 2′-OMe modification, preferably measured as a percentage of the total nucleotides of both the first and second strands;
(x) no more than 20%, (such as no more than 15% or no more than 10%) of 2′-F modifications on the first and/or second strand, as a percentage of the total nucleotides of both strands;
(xi) the terminal nucleotide at the 3′ end of at least one of the first strand and the second strand is an inverted nucleotide and is attached to the adjacent nucleotide via the 3′ carbon of the terminal nucleotide and the 3′ carbon of the adjacent nucleotide and/or the terminal nucleotide at the 5′ end of at least one of the first strand and the second strand is an inverted nucleotide and is attached to the adjacent nucleotide via the 5′ carbon of the terminal nucleotide and the 5′ carbon of the adjacent nucleotide, or wherein the nucleic acid comprises a phosphorodithioate linkage;
(xii) the nucleic acid is for inhibiting expression of TMPRSS6.

Modified nucleic acids, particularly nucleic acids or RNAs of conjugates, as used herein, can include one or more of:
(i) alteration, e.g., replacement, of one or both of the non-linking phosphate oxygens and/or of one or more of the linking phosphate oxygens (referred to as linking even if at the 5′ and 3′ terminus of the nucleic acid of the invention);
(ii) alteration, e.g., replacement, of a constituent of the ribose sugar, e.g., of the 2′ hydroxyl on the ribose sugar;
(iii) replacement of the phosphate moiety with “dephospho” linkers;
(iv) modification or replacement of a naturally occurring base;
(v) replacement or modification of the ribose-phosphate backbone; and
(vi) modification of the 3′end or 5′end of the first strand and/or the second strand, e.g., removal, modification or replacement of a terminal phosphate group or conjugation of a moiety, e.g., a fluorescently labelled moiety, to either the 3′ or 5′end one or both strands.

The terms replacement, modification, alteration, indicate a difference from a naturally occurring molecule.

Some representative modified nucleic acid sequences of the present invention are shown in the examples. These examples are meant to be representative and not limiting.

In one aspect of the nucleic acid, at least nucleotides 2 and 14 of the first strand are modified, preferably by a first common modification, the nucleotides being numbered consecutively starting with nucleotide number 1 at the 5′ end of the first strand. The first modification is preferably 2′-F.

In one aspect, at least one, several or preferably all the even-numbered nucleotides of the first strand are modified, preferably by a first common modification, the nucleotides being numbered consecutively starting with nucleotide number 1 at the 5′ end of the first strand. The first modification is preferably 2′-F.

In one aspect, at least one, several or preferably all the odd-numbered nucleotides of the first strand are modified, the nucleotides being numbered consecutively starting with nucleotide number 1 at the 5′ end of the first strand. Preferably, they are modified by a second modification. This second modification is preferably different from the first modification if the nucleic acid also comprises a first modification, for example of nucleotides 2 and 14 or of one, several or all of the even-numbered nucleotides of the first strand. The first modification is preferably 2′-F and the second modification is preferably 2′-OMe.

In one aspect, at least one, several or preferably all the nucleotides of the second strand in a position corresponding to an even-numbered nucleotide of the first strand are modified, preferably by a third modification. Preferably nucleotides 2 and 14 or all the even numbered nucleotides of the first strand are modified with a first modification in the same nucleic acid. In addition, or alternatively, the odd-numbered nucleotides of the first strand are modified with a second modification. Preferably, the third modification is different from the first modification and/or the third modification is the same as the second modification. The first modification is preferably 2′-F and the second and third modifications are preferably 2′-OMe. The nucleotides on the first strand are numbered consecutively starting with nucleotide number 1 at the 5′ end of the first strand.

A nucleotide of the second strand that is in a position corresponding for example to an even-numbered nucleotide of the first strand is a nucleotide of the second strand that is base paired to an even-numbered nucleotide of the first strand.

In one aspect, at least one, several or preferably all the nucleotides of the second strand in a position corresponding to an odd-numbered nucleotide of the first strand are modified, preferably by a fourth modification. Preferably nucleotides 2 and 14 or all the even numbered nucleotides of the first strand are modified with a first modification in the same nucleic acid. In addition, or alternatively, the odd-numbered nucleotides of the first strand are modified with a second modification. In addition, or alternatively, all the nucleotides of the second strand in a position corresponding to an even-numbered nucleotide of the first strand are modified with a third modification. The fourth modification is preferably different from the second modification and preferably different from the third modification and the fourth modification is preferably the same as the first modification. The second and third modification are preferably the same. The first and the fourth modification are preferably a 2′-OMe modification and the second and third modification are preferably a 2′-F modification. The nucleotides on the first strand are numbered consecutively starting with nucleotide number 1 at the 5′ end of the first strand.

In one aspect of the nucleic acid, the nucleotide/nucleotides of the second strand in a position corresponding to nucleotide 11 or nucleotide 13 or nucleotides 11 and 13 or nucleotides 11-13 of the first strand is/are modified by a fourth modification. Preferably, all the nucleotides of the second strand other than the nucleotide/nucleotides in a position corresponding to nucleotide 11 or nucleotide 13 or nucleotides 11 and 13 or nucleotides 11-13 of the first strand is/are modified by a third modification. Preferably nucleotides 2 and 14 or all the even numbered nucleotides of the first strand are modified with a first modification in the same nucleic acid. In addition, or alternatively, the odd-numbered nucleotides of the first strand are modified with a second modification. The fourth modification is preferably different from the second modification and preferably different from the third modification and the fourth modification is preferably the same as the first modification. The second and third modification are preferably the same. The first and the fourth modification are preferably a 2′-OMe modification and the second and third modification are preferably a 2′-F modification. The nucleotides on the first strand are numbered consecutively starting with nucleotide number 1 at the 5′ end of the first strand.

In one aspect of the nucleic acid, all the even-numbered nucleotides of the first strand are modified by a first modification, all the odd-numbered nucleotides of the first strand are modified by a second modification, all the nucleotides of the second strand in a position corresponding to an even-numbered nucleotide of the first strand are modified by a third modification, all the nucleotides of the second strand in a position corresponding to an odd-numbered nucleotide of the first strand are modified by a fourth modification, wherein the first and fourth modification are 2′-F and the second and third modification are 2′-OMe.

In one aspect of the nucleic acid, all the even-numbered nucleotides of the first strand are modified by a first modification, all the odd-numbered nucleotides of the first strand are modified by a second modification, all the nucleotides of the second strand in positions corresponding to nucleotides 11-13 of the first strand are modified by a fourth modification, all the nucleotides of the second strand other than the nucleotides corresponding to nucleotides 11-13 of the first strand are modified by a third modification, wherein the first and fourth modification are 2′-F and the second and third modification are 2′-OMe. Preferably in this aspect, the 3′ terminal nucleotide of the second strand is an inverted RNA nucleotide (ie the nucleotide is linked to the 3′ end of the strand through its 3′ carbon, rather than through its 5′ carbon as would normally be the case). When the 3′ terminal nucleotide of the second strand is an inverted RNA nucleotide, the inverted RNA nucleotide is preferably an unmodified nucleotide in the sense that it does not comprise any modifications compared to the natural nucleotide counterpart. Specifically, the inverted RNA nucleotide is preferably a 2′-OH nucleotide.

One aspect is a nucleic acid as disclosed herein, wherein the nucleotides at positions 2 and 14 from the 5′ end of the first strand are not modified with a 2′-OMe modification, and the nucleotide/nucleotides on the second strand which correspond to position 11 or position 13 or positions 11 and 13 or positions 11, 12 and 13 of the first strand is/are not modified with a 2′-OMe modification (in other words, they are unmodified nucleotides or are nucleotides modified with a modification other than 2′-OMe).

In one aspect the nucleotide on the second strand which corresponds to position 13 of the first strand is the nucleotide that forms a base pair with position 13 of the first strand.

In one aspect the nucleotide on the second strand which corresponds to position 11 of the first strand is the nucleotide that forms a base pair with position 11 of the first strand.

In one aspect the nucleotide on the second strand which corresponds to position 12 of the first strand is the nucleotide that forms a base pair with position 12 of the first strand.

For example, in a 19-mer nucleic acid which is double-stranded and blunt-ended, position 13 of the first strand would pair with position 7 of the second strand. Position 11 of the first strand would pair with position 9 of the second strand. This nomenclature may be applied to other positions of the second strand.

In one aspect, in the case of a partially complementary first and second strand, the nucleotide on the second strand that “corresponds to” a position on the first strand may not necessarily form a base pair if that position is the position in which there is a mismatch, but the principle of the nomenclature still applies.

One aspect is a nucleic acid as disclosed herein, wherein the nucleotides at positions 2 and 14 from the 5′ end of the first strand are not modified with a 2′-OMe modification, and the nucleotides on the second strand which correspond to position 11, or 13, or 11 and 13, or 11-13 of the first strand are modified with a 2′-F modification.

One aspect is a nucleic acid as disclosed herein, wherein the nucleotides at positions 2 and 14 from the 5′ end of the first strand are modified with a 2′-F modification, and the nucleotides on the second strand which correspond to position 11, or 13, or 11 and 13, or 11-13 of the first strand are not modified with a 2′-OMe modification.

One aspect is a nucleic acid as disclosed herein, wherein the nucleotides at positions 2 and 14 from the 5′ end of the first strand are modified with a 2′-F modification, and the nucleotides on the second strand which correspond to position 11, or 13, or 11 and 13, or 11-13 of the first strand are modified with a 2′-F modification.

One aspect is a nucleic acid as disclosed herein, wherein all nucleotides are modified with a 2′-OMe modification except positions 2 and 14 from the 5′ end of the first strand and the nucleotides on the second strand which correspond to position 11, or 13, or 11 and 13, or 11-13 of the first strand. Preferably the nucleotides that are not modified with 2′-OMe are modified with fluoro at the 2′ position (2′-F modification).

Preferred is a nucleic acid as disclosed herein wherein all nucleotides of the nucleic acid are modified at the 2′ position of the sugar. Preferably these nucleotides are modified with a 2′-F modification where the modification is not a 2′-OMe modification.

In one aspect the nucleic acid is modified on the first strand with alternating 2′-OMe modifications and 2-F modifications, and positions 2 and 14 (starting from the 5′ end) are modified with 2′-F.

Preferably the second strand is modified with 2′-F modifications at nucleotides on the second strand which correspond to position 11, or 13, or 11 and 13, or 11-13 of the first strand. Preferably the second strand is modified with 2′-F modifications at positions 11-13 counting from the 3′ end starting at the first position of the complementary (double-stranded) region, and the remaining modifications are naturally occurring modifications, preferably 2′-OMe.

In one aspect of the nucleic acid, each of the nucleotides of the first strand and of the second strand is a modified nucleotide.

In one aspect, at least one nucleotide of the first and/or second strand is a modified nucleotide, wherein if the first strand comprises at least one modified nucleotide:
(i) at least one or both of the nucleotides 2 and 14 of the first strand is/are modified by a first modification; and/or
(ii) at least one, several, or all the even-numbered nucleotides of the first strand is/are modified by a first modification; and/or
(iii) at least one, several, or all the odd-numbered nucleotides of the first strand is/are modified by a second modification; and/or

wherein if the second strand comprises at least one modified nucleotide:
(iv) at least one, several, or all the nucleotides of the second strand in a position corresponding to an even-numbered nucleotide of the first strand is/are modified by a third modification; and/or
(v) at least one, several, or all the nucleotides of the second strand in a position corresponding to an odd-numbered nucleotide of the first strand is/are modified by a fourth modification; and/or
(vi) at least one, several, or all the nucleotides of the second strand in a position corresponding to nucleotide 11 or nucleotide 13 or nucleotides 11 and 13 or nucleotides 11-13 of the first strand is/are modified by a fourth modification; and/or
(vii) at least one, several, or all the nucleotides of the second strand in a position other than the position corresponding to nucleotide 11 or nucleotide 13 or nucleotides 11 and 13 or nucleotides 11-13 of the first strand is/are modified by a third modification;

wherein the nucleotides on the first strand are numbered consecutively starting with nucleotide number 1 at the 5′ end of the first strand;

wherein the modifications are preferably at least one of the following:
(a) the first modification is preferably different from the second and from the third modification;
(b) the first modification is preferably the same as the fourth modification;
(c) the second and the third modification are preferably the same modification;
(d) the first modification is preferably a 2′-F modification;
(e) the second modification is preferably a 2′-OMe modification;
(f) the third modification is preferably a 2′-OMe modification; and/or
(g) the fourth modification is preferably a 2′-F modification; and

wherein optionally the nucleic acid is conjugated to a ligand.

11 Flanking Groups

In one aspect, the antisense duplex region comprises a plurality of groups of modified nucleotides, referred to herein as “modified groups”, wherein each modified group consists of one or more identically modified nucleotides, wherein each modified group is flanked on one or both sides by a second group of nucleotides, referred to herein as “flanking groups”, wherein each said flanking group consists of one or more nucleotides that are either unmodified or modified in a manner different from the nucleotides of said modified group. In one embodiment, each modified group in the antisense duplex region is identical, i.e., each modified group consists of an equal number of identically modified nucleotides. In another embodiment, each flanking group has an equal number of nucleotides. In another embodiment, each flanking group is identical. In another embodiment, the nucleotides of said modified groups in the antisense duplex region comprise a modified base. In another embodiment, the nucleotides of said modified groups comprise a modified phosphate backbone. In another embodiment, the nucleotides of said modified groups comprise a modified 2′ position.

In another aspect, the sense duplex region comprises a plurality of groups of modified groups, wherein each modified group consists of one or more identically modified nucleotides, wherein each modified group is flanked on one or both sides by a flanking group, wherein each said flanking group consists of one or more nucleotides that are either unmodified or modified in a manner different from the nucleotides of said modified group. In one embodiment, each modified group in the sense duplex region is identical. In another embodiment, each flanking group has an equal number of nucleotides. In another embodiment, each flanking group is identical. In another embodiment, the nucleotides of said modified groups in the sense duplex region comprise a modified base. In another embodiment, the nucleotides of said modified groups comprise a modified phosphate backbone. In another embodiment, the nucleotides of said modified groups comprise a modified 2′ position.

In another aspect, the antisense duplex region and the sense duplex region each comprise a plurality of modified groups, wherein each modified group consists of one or more identically modified nucleotides, wherein each modified group is flanked on one or both sides by a flanking group, wherein each said flanking group consists of one or more nucleotides that are either unmodified or modified in a manner different from the nucleotides of said modified group. In one embodiment, each modified group in the antisense duplex region and the sense duplex region are identical. In another embodiment, each flanking group in the antisense duplex region and the sense duplex region each have an equal number of nucleotides. In another embodiment, each flanking group in the antisense duplex region and in the sense duplex region are identical. In another embodiment, the nucleotides of said modified groups in the antisense duplex region and the sense duplex region each comprise the same modified groups and the same flanking groups. In another embodiment, the nucleotides of said modified groups in the antisense duplex region and the sense duplex region each comprise a modified base. In another embodiment, the nucleotides of said modified groups in the antisense duplex region and the sense duplex region each comprise a modified phosphate backbone. In another embodiment, the nucleotides of said modified groups in the antisense duplex region and the sense duplex region each comprise a modified 2′ position.

In one aspect, the antisense strand comprises a plurality of groups of modified nucleotides, referred to herein as “modified groups”, wherein each modified group consists of one or more identically modified nucleotides, wherein each modified group is flanked on one or both sides by a second group of nucleotides, referred to herein as “flanking groups”, wherein each said flanking group consists of one or more nucleotides that are either unmodified or modified in a manner different from the nucleotides of said modified group. In one embodiment, each modified group in the antisense strand is identical, i.e., each modified group consists of an equal number of identically modified nucleotides. In another embodiment, each flanking group has an equal number of nucleotides. In another embodiment, each flanking group is identical. In another embodiment, the nucleotides of said modified groups in the antisense strand comprise a modified base. In another embodiment, the nucleotides of said modified groups comprise a modified phosphate backbone. In another embodiment, the nucleotides of said modified groups comprise a modified 2′ position.

In another aspect, the sense strand comprises a plurality of groups of modified groups, wherein each modified group consists of one or more identically modified nucleotides, wherein each modified group is flanked on one or both sides by a flanking group, wherein each said flanking group consists of one or more nucleotides that are either unmodified or modified in a manner different from the nucleotides of said modified group. In one embodiment, each modified group in the sense strand is identical. In another embodiment, each flanking group has an equal number of nucleotides. In another embodiment, each flanking group is identical. In another embodiment, the nucleotides of said modified groups in the sense strand comprise a modified base. In another embodiment, the nucleotides of said modified groups comprise a modified phosphate backbone. In another embodiment, the nucleotides of said modified groups comprise a modified 2′ position.

In another aspect, the antisense strand and the sense strand each comprise a plurality of modified groups, wherein each modified group consists of one or more identically modified nucleotides, wherein each modified group is flanked on one or both sides by a flanking group, wherein each said flanking group consists of one or more nucleotides that are either unmodified or modified in a manner different from the nucleotides of said modified group. In one embodiment, each modified group in the antisense strand and the sense strand are identical. In another embodiment, each flanking group in the antisense strand and the sense strand each have an equal number of nucleotides. In another embodiment, each flanking group in the antisense strand and in the sense strand are identical. In another embodiment, the nucleotides of said modified groups in the antisense strand and the sense strand each comprise the same modified groups and the same flanking groups. In another embodiment, the nucleotides of said modified groups in the antisense strand and the sense strand each comprise a modified base. In another embodiment, the nucleotides of said modified groups in the antisense strand and the sense strand each comprise a modified phosphate backbone. In another embodiment, the nucleotides of said modified groups in the antisense strand and the sense strand each comprise a modified 2′ position.

In another aspect, the modified groups and the flanking groups form a regular pattern on the antisense stand. In another aspect, the modified groups and the flanking groups form a regular pattern on the sense strand. In one embodiment, the modified groups and the flanking groups form a regular pattern on the both the antisense strand and the sense strand. In another embodiment, the modified groups and the flanking groups form a regular pattern on the antisense duplex region. In another aspect, the modified groups and the flanking groups form a regular pattern on the sense duplex region. In one embodiment, the modified groups and the flanking groups form a regular pattern on the both the antisense duplex region and the sense duplex region.

In another aspect, the pattern is a spatial or positional pattern. A spatial or positional pattern means that (a) nucleotide(s) are modified depending on their position within the nucleotide sequence of a double-stranded portion. Accordingly, it does not matter whether the nucleotide to be modified is a pyrimidine or a purine. Rather the position of a modified nucleotide is dependent upon: (a) its numbered position on a strand of nucleic acid, wherein the nucleotides are numbered from the 5′-end to the 3′-end with the 5′-end nucleotide of the strand being position one (both the antisense strand and sense strand are numbered from their respective 5′-end nucleotide), or (b) the position of the modified group relative to a flanking group. Thus, according to this embodiment, the modification pattern will always be the same, regardless of the sequence which is to be modified.

In one embodiment, each modified group on both the antisense strand and the sense strand is identical. In one embodiment, each modified group on both the antisense duplex region and the sense duplex region is identical. In another embodiment, each modified group and each flanking group on both the antisense strand and the sense strand are identical. In one embodiment, each modified group and each flanking group on both the antisense duplex region and the sense duplex region are identical.

In one embodiment, each modified group, each modified group position, each flanking group and each flanking group position on both the antisense strand and the sense strand are identical. In one embodiment, each modified group, each modified group position, each flanking group and each flanking group position on both the antisense duplex region and the sense duplex region are identical. In another embodiment, the modified groups on the antisense strand are complementary with the modified groups on the sense strand (the modified groups on the antisense strand and the sense strand are perfectly aligned across from one another). In another embodiment, there are no mismatches in the modified groups such that each modified group on the antisense strand is base paired with each modified group on the sense strand.

In another embodiment, each modified group on the sense strand is shifted by 1, 2, 3, 4 or 5 nucleotides relative to the modified groups on the antisense strand. For example, if each modified group on the sense strand is shifted by one nucleotide or one group of nucleotides and a modified group started at position one on the antisense strand, a modified group on the sense strand would begin at position two. In another embodiment, the modified groups of the antisense strand do not overlap the modified groups of the sense strand, i.e., no nucleotide of a modified group on the antisense strand is base paired with a nucleotide of a modified group on the sense strand. In one embodiment, deoxyribonucleotides at an end of a strand of nucleic acid are not considered when determining a position of a modified group, i.e., the positional numbering begins with the first ribonucleotide or modified ribonucleotide. In another embodiment, abasic nucleotides at an end of a strand of nucleic acid are not considered when determining a position of a modified group.

In one aspect, a modified group comprises a 5′-end nucleotide of either or both of the antisense strand and the sense strand. In another embodiment, a flanking group comprises the 5′-end nucleotide of either or both of the antisense strand and the sense strand. In another embodiment, the 5′-end nucleotide of either or both of the antisense strand and the sense strand is unmodified. In another embodiment, a modified group comprises the 5′-end nucleotide of either or both of the antisense duplex region and sense duplex region. In another embodiment, a flanking group comprises the 5′-end nucleotide of either or both of the antisense duplex region or the sense duplex region. In another embodiment, the 5′-end nucleotide of either or both of the antisense duplex region or the sense duplex region is unmodified. In one embodiment, the modification at the 2′ position is selected from the group comprising amino, fluoro, methoxy, alkoxy and C1-C3-alkyl. In another embodiment, the modification may be selected from 2′-O-methyl (2′-OMe), 2′-O-alkyl, and 2′-O—(C1-C3-alkyl). In another embodiment, the modification at the 2′ position is 2′-OMe.

Inverted Nucleotides

The nucleic acids of the invention may include one or more inverted nucleotides, for example inverted thymidine or inverted adenine (for example see Takei, et al., 2002. JBC 277 (26):23800-06). In particular, the nucleic acids of the invention may comprise a modification wherein the terminal nucleotide at the 3′ end of at least one of the first strand and the second strand is an inverted nucleotide and is attached to the adjacent nucleotide via the 3′ carbon of the terminal nucleotide and the 3′ carbon of the adjacent nucleotide and/or the terminal nucleotide at the 5′ end of at least one of the first strand and the second strand is an inverted nucleotide and is attached to the adjacent nucleotide via the 5′ carbon of the terminal nucleotide and the 5′ carbon of the adjacent nucleotide. For example, the inverted nucleotide at the 3′ end of at least one of the first strand and the second strand and/or the inverted nucleotide at the 5′ end of at least one of the first strand and the second strand is a purine, such as an adenine.

The nucleic acid of the invention may comprise an inverted RNA nucleotide at one or several of the strand ends. Such inverted nucleotides provide stability to the nucleic acid. Preferably, the nucleic acid comprises at least an inverted nucleotide at the 3′ end of the first and/or the second strand and/or at the 5′ end of the second strand. More preferably, the nucleic acid comprises an inverted nucleotide at the 3′ end of the second strand. Most preferably, the nucleic acid comprises an inverted RNA nucleotide at the 3′ end of the second strand and this nucleotide is preferably an inverted A. An inverted nucleotide is a nucleotide that is linked to the 3′ end of a nucleic acid through its 3′ carbon, rather than its 5′ carbon as would normally be the case or is linked to the 5′ end of a nucleic acid through its 5′ carbon, rather than its 3′ carbon as would normally be the case. The inverted nucleotide is preferably present at an end of a strand not as an overhang but opposite a corresponding nucleotide in the other strand. Accordingly, the nucleic acid is preferably blunt-ended at the end that comprises the inverted RNA nucleotide. An inverted RNA nucleotide being present at the end of a strand preferably means that the last nucleotide at this end of the strand is the inverted RNA nucleotide. A nucleic acid with such a nucleotide is stable and easy to synthesise. The inverted RNA nucleotide is preferably an unmodified nucleotide in the sense that it does not comprise any modifications compared to the natural nucleotide counterpart. Specifically, the inverted RNA nucleotide is preferably a 2′-OH nucleotide.

In further embodiments of the invention, the invention relates to any nucleic acid, conjugated nucleic acid, nucleic acid for use, method, composition or use according to any disclosure herein, wherein the terminal nucleotide at the 3′ end of at least one of the first strand and the second strand is an inverted nucleotide and is attached to the adjacent nucleotide via the 3′ carbon of the terminal nucleotide and the 3′ carbon of the adjacent nucleotide and/or the terminal nucleotide at the 5′ end of at least one of the first strand and the second strand is an inverted nucleotide and is attached to the adjacent nucleotide via the 5′ carbon of the terminal nucleotide and the 5′ carbon of the adjacent nucleotide,

optionally wherein the 3′ and/or 5′ inverted nucleotide of the first and/or second strand is attached to the adjacent nucleotide via a phosphate group by way of a phosphodiester linkage; or

the 3′ and/or 5′ inverted nucleotide of the first and/or second strand is attached to the adjacent nucleotide via a phosphorothioate group; or

the 3′ and/or 5′ inverted nucleotide of the first and/or second strand is attached to the adjacent nucleotide via a phosphorodithioate group.

Formulations for Delivery of the Conjugates of the Present Invention

Conjugates of the present invention (such as siRNAs) can be delivered to cells, both in vitro and in vivo, by a variety of methods known to those skilled in the art, including direct contact with cells (“naked” RNA) or by combination with one or more agents that facilitate targeting or delivery into cells. Such agents and methods include lipoplexes, liposomes, iontophoresis, hydrogels, cyclodextrins, nanocapsules, micro- and nanospheres and proteinaceous vectors. The nucleic acid/vehicle combination may be locally delivered in vivo by direct injection or by use of an infusion pump. Conjugates of the invention (such as siRNAs) can be delivered in vivo by various means including intravenous subcutaneous, intramuscular or intradermal injection or inhalation. The molecules can be used as pharmaceutical agents. Preferably, pharmaceutical agents prevent, modulate the occurrence, treat or alleviate a symptom of a disease state in a subject.

Conjugates of the invention (such as siRNAs) may be formulated as pharmaceutical compositions which may further comprise a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Conjugates of the invention (such as siRNAs) may be used as medicaments or as diagnostic agents, alone or in combination with other agents. For example, one or more conjugates of the present invention can be combined with a delivery vehicle (e.g., liposomes) and excipients, such as carriers, diluents. Other agents such as preservatives and stabilizers can also be added. Methods for the delivery of nucleic acid conjugates are known in the art and within the knowledge of the person skilled in the art.

Pharmaceutically acceptable compositions may comprise a therapeutically-effective amount of one or more conjugates of the invention (such as siRNAs), taken alone or formulated with one or more pharmaceutically acceptable carriers, excipient and/or diluents.

Examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatine; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerine, sorbitol, mannitol and polyethylene glycol; (12) esters, such as ethyl oleate and ethyl laurate; (13) agar; (14) buffering agents, such as magnesium hydroxide and aluminium hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

Stabilisers may be agents that stabilise the conjugates of the invention (such as siRNAs), for example a protein that can complex with the nucleic acid, chelators (e.g. EDTA), salts, RNAse inhibitors, and DNAse inhibitors.

In some cases it is desirable to slow the absorption of the drug from subcutaneous or intramuscular injection in order to prolong the effect of a drug. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The conjugates of the present invention can also be administered in combination with other therapeutic compounds, either administrated separately or simultaneously, e.g., as a combined unit dose. In one embodiment, the invention includes a pharmaceutical composition comprising one or more conjugates of the present invention in a physiologically/pharmaceutically acceptable excipient, such as a stabilizer, preservative, diluent, buffer, and the like. Conjugates of the invention (such as siRNAs) may, for example be formulated in water for example water for injection, saline or phosphate buffered saline.

The pharmaceutical composition may be a sterile injectable aqueous suspension or solution, or in a lyophilized form. In one embodiment, the pharmaceutical composition may comprise lyophilized lipoplexes or an aqueous suspension of lipoplexes. The lipoplexes preferably comprises a RNA of the present invention. Such lipoplexes may be used to deliver the RNA of the invention to a target cell either in vitro or in vivo.

The nucleic acid or conjugate as described herein may be formulated with a lipid in the form of a liposome. Such a formulation may be described in the art as a lipoplex. The formulation with a lipid/liposome may be used to assist with delivery of the nucleic acid of the invention to the target cells. The lipid delivery system herein described may be used as an alternative to a conjugated ligand. The modifications herein described may be present when using a nucleic acid of the invention with a lipid delivery system or with a ligand conjugate delivery system.

The invention also provides a composition comprising a nucleic acid or conjugated nucleic acid as defined herein and a physiologically acceptable excipient. The composition can comprise the following excipients:
i) a cationic lipid, or a pharmaceutically acceptable salt thereof;
ii) a steroid;
iii) a phosphatidylethanolamine phospholipid;
iv) a PEGylated lipid.

The content of the cationic lipid component in the composition may be from about 55 mol % to about 65 mol % of the overall lipid content of the lipid formulation, preferably about 59 mol % of the overall lipid content of the lipid composition.

The cationic lipid may be an amino cationic lipid.

The cationic lipid may have the formula:

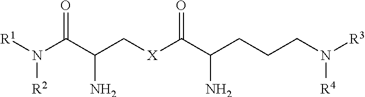

or a pharmaceutically acceptable salt thereof, wherein:

X represents O, S or NH;

R¹ and R² each independently represents a C₄-C₂₂ linear or branched alkyl chain or a C₄-C₂₂ linear or branched alkenyl chain with one or more double bonds, wherein the alkyl or alkenyl chain optionally contains an intervening ester, amide or disulfide;

when X represents S or NH, R³ and R⁴ each independently represent hydrogen, methyl, ethyl, a mono- or polyamine moiety, or R³ and R⁴ together form a heterocyclyl ring;

when X represents O, R³ and R⁴ each independently represent hydrogen, methyl, ethyl, a mono- or polyamine moiety, or R³ and R⁴ together form a heterocyclyl ring, or R³ represents hydrogen and R⁴ represents C(NH)(NH₂).

The cationic lipid may have the formula:

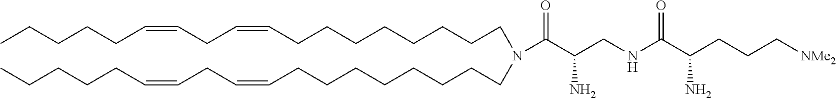 [SMILES: CCCCC/C=C\C/C=C\CCCCCCCCN(CCCCCCCC/C=C\C/C=C\CCCCC)C(=O)[C@@H](N)CNC(=O)[C@@H](N)CCCN(C)C]

or a pharmaceutically acceptable salt thereof.

The cationic lipid may have the formula:

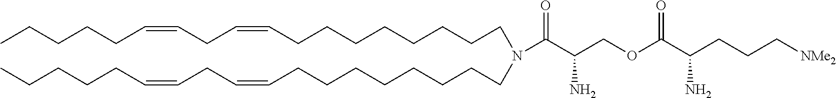 [SMILES: CCCCC/C=C\C/C=C\CCCCCCCCN(CCCCCCCC/C=C\C/C=C\CCCCC)C(=O)[C@@H](N)COC(=O)[C@@H](N)CCCN(C)C]

or a pharmaceutically acceptable salt thereof.

The content of the cationic lipid component may be from about 55 mol % to about 65 mol % of the overall lipid content of the formulation. In particular, the cationic lipid component is about 59 mol % of the overall lipid content of the formulation.

The formulations further comprise a steroid. the steroid may be cholesterol. The content of the steroid may be from about 26 mol % to about 35 mol % of the overall lipid content of the lipid formulation. More particularly, the content of steroid may be about 30 mol % of the overall lipid content of the lipid formulation.

The phosphatidylethanolamine phospholipid may be selected from group consisting of 1,2-diphytanoyl-sn-glycero-3-phosphoethanolamine (DPhyPE), 1,2-dioleoyl-sn-glycero-3-phosphoethanolamine (DOPE), 1,2-distearoyl-sn-glycero-3-phosphoethanolamine (DSPE), 1,2-Dilauroyl-sn-glycero-3-phosphoethanolamine (DLPE), 1,2-Dimyristoyl-sn-glycero-3-phosphoethanolamine (DMPE), 1,2-Dipalmitoyl-sn-glycero-3-phosphoethanolamine (DPPE), 1,2-Dilinoleoyl-sn-glycero-3-phosphoethanolamine (DLoPE), 1-Palmitoyl-2-oleoyl-sn-glycero-3-phosphoethanolamine (POPE), 1,2-Dierucoyl-sn-glycero-3-phosphoethanolamine (DEPE), 1,2-Disqualeoyl-sn-glycero-3-phosphoethanolamine (DSQPE) and 1-Stearoyl-2-inoleoyl-sn-glycero-3-phosphoethanolamine (SLPE). The content of the phospholipid may be about 10 mol % of the overall lipid content of the formulation.

The PEGylated lipid may be selected from the group consisting of 1,2-dimyristoyl-sn-glycerol, methoxypolyethylene glycol (DMG-PEG) and C16-Ceramide-PEG. The content of the PEGylated lipid may be about 1 to 5 mol % of the overall lipid content of the formulation.

The content of the cationic lipid component in the formulation may be from about 55 mol % to about 65 mol % of the overall lipid content of the lipid formulation, preferably about 59 mol % of the overall lipid content of the lipid formulation.

Neutral liposome compositions may be formed from, for example, dimyristoyl phosphatidylcholine (DMPC) or dipalmitoyl phosphatidylcholine (DPPC). Anionic liposome compositions may be formed from dimyristoyl phosphatidylglycerol, while anionic fusogenic liposomes may be formed primarily from dioleoyl phosphatidylethanolamine (DOPE). Another type of liposomal composition may be formed from phosphatidylcholine (PC) such as, for example, soybean PC, and egg PC.

Another type is formed from mixtures of phospholipid and/or phosphatidylcholine and/or cholesterol.

A positively charged synthetic cationic lipid, N—[I-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells. DOTMA analogues can also be used to form liposomes.

The formulation may have a molar ratio of the components of i):ii):iii):iv) selected from 55:34:10:1; 56:33:10:1; 57:32:10:1; 58:31:10:1; 59:30:10:1; 60:29:10:1; 61:28:10:1; 62:27:10:1; 63:26:10:1; 64:25:10:1; and 65:24:10:1.

The composition may comprise a cationic lipid having the structure

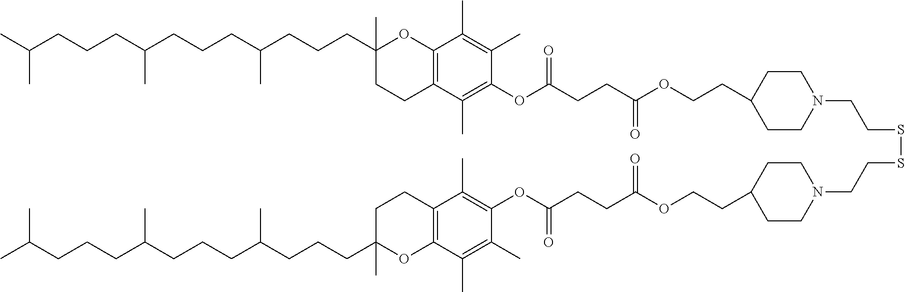 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

a steroid having the structure

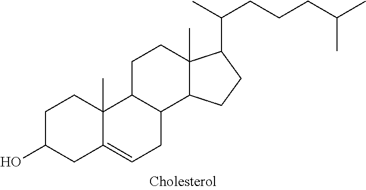 [SMILES: CC(C)CCCC(C)C1CCC2C3CC=C4CC(O)CCC4(C)C3CCC12C]

a phosphatidylethanolamine phospholipid having the structure

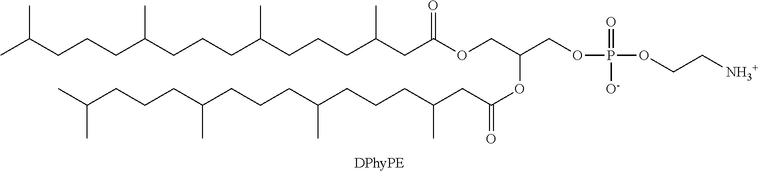 [SMILES: CC(C)CCCC(C)CCCC(C)CCCC(C)CC(=O)OCC(COP(=O)([O-])OCC[NH3+])OC(=O)CC(C)CCCC(C)CCCC(C)CCCC(C)C]

and a PEGylated lipid having the structure

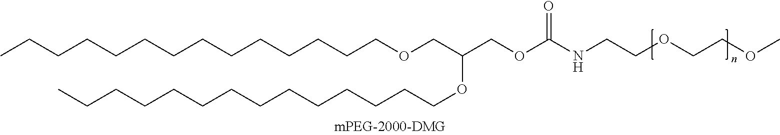

Also provided is a nucleic acid or conjugated nucleic acid according to any aspect of the invention for use in the treatment of a disease or disorder and/or in the manufacture of a medicament for treating a disease or disorder.

The invention provides a method of treating a disease or disorder comprising administration of a composition comprising a nucleic acid or conjugated nucleic acid according to any aspect of the invention to an individual in need of treatment. The nucleic acid may be administered to the subject subcutaneously, intravenously or using any other application routes such as oral, rectal or intraperitoneal.

A method of making a nucleic acid or conjugated nucleic acid according to the invention is also included.

Surfactants

Compositions comprising the conjugates of the invention (such as siRNAs) may include a surfactant. In one embodiment, the conjugate of the invention (such as siRNAs) is formulated as an emulsion that includes a surfactant.

A surfactant that is not ionized is a non-ionic surfactant. Examples include non-ionic esters, such as ethylene glycol esters, propylene glycol esters, glyceryl esters etc., nonionic alkanolamides, and ethers such as fatty alcohol ethoxylates, propoxylated alcohols, and ethoxylated/propoxylated block polymers.

A surfactant that carries a negative charge when dissolved or dispersed in water is an anionic surfactant. Examples include carboxylates, such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates.

A surfactant that carries a positive charge when dissolved or dispersed in water is a cationic surfactant. Examples include quaternary ammonium salts and ethoxylated amines.

A surfactant that has the ability to carry either a positive or negative charge is an amphoteric surfactant. Examples include acrylic acid derivatives, substituted alkylamides, N-alkylbetaines and phosphatides.

Micelles and Other Membranous Formulations

“Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic. A micelle may be formed by mixing an aqueous solution of the nucleic acid, an alkali metal alkyl sulphate, and at least one micelle forming compound.

Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerine, polyglycerol, lysine, polylysine, triolein, polyoxyethylene ethers and analogues thereof, polidocanol alkyl ethers and analogues thereof, chenodeoxycholate, deoxycholate, and mixtures thereof.

Phenol and/or m-cresol may be added to the mixed micellar composition to act as a stabiliser and preservative. An isotonic agent such as glycerine may as be added.

Particles

A composition comprising conjugate of the invention (such as siRNAs) may be incorporated into a particle such as a microparticle. Microparticles can be produced by spray-drying, lyophilisation, evaporation, fluid bed drying, vacuum drying, or a combination of these methods.

Dosage

Dosage levels for the conjugates of the present invention can be determined by those skilled in the art by routine experimentation. In one embodiment, a unit dose may contain between about 0.01 mg/kg and about 100 mg/kg body weight of RNA. Alternatively, the dose can be from 10 mg/kg to 25 mg/kg body weight, or 1 mg/kg to 10 mg/kg body weight, or 0.05 mg/kg to 5 mg/kg body weight, or 0.1 mg/kg to 5 mg/kg body weight, or 0.1 mg/kg tol mg/kg body weight, or 0.1 mg/kg to 0.5 mg/kg body weight, or 0.5 mg/kg to 1 mg/kg body weight. Dosage levels may also be calculated via other parameters such as, e.g., body surface area.

The conjugates or compositions and medicaments of the present invention may be administered to a mammalian subject in a pharmaceutically effective dose. The mammal may be selected from humans, dogs, cats, horses, cattle, pig, goat, sheep, mouse, rat, hamster and guinea pig.

In one embodiment, a subject is administered an initial dose and one or more maintenance doses of a conjugate of the invention (such as siRNAs). The maintenance dose or doses can be the same or lower than the initial dose, e.g., one-half less of the initial dose. The maintenance doses are, for example, administered no more than once every 2, 5, 10, or 30 days. The treatment regimen may last for a period of time which will vary depending upon the nature of the particular disease, its severity and the overall condition of the patient.

Routes of Delivery

The conjugates of the present invention can be delivered to a subject by a variety of routes. Exemplary routes include: subcuteanous, intravenous, topical, rectal, anal, vaginal, nasal, pulmonary, ocular.

The conjugates of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The pharmaceutical composition may be specially formulated for administration in solid or liquid form. The composition may be formulated for oral administration, parenteral administration (including, for example, subcutaneous, intramuscular, intravenous, or epidural injection), topical application, intravaginal or intrarectal administration, sublingual administration, ocular administration, transdermal administration, or nasal administration. Delivery using subcutaneous or intravenous methods are preferred.

The route and site of administration may be chosen to enhance targeting. For example, to target muscle cells, intramuscular injection into the muscles of interest would be a logical choice. Lung cells might be targeted by administering the conjugate in aerosol form. The vascular endothelial cells could be targeted by coating a balloon catheter with the RNA and mechanically introducing the iRNA.

Uses and Methods

The conjugates of the present invention may have use in medicine. In particular, the conjugates of the present invention may be used for the treatment of liver disease, genetic disease, haemophilia and bleeding disorder, liver fibrosis, non alcoholic steatohepatitis (NASH), non alcoholic fatty liver disease (NAFLD), viral hepatitis, rare diseases (e.g. acromegaly), metabolic diseases (e.g. hypercholesterolemia, dyslipidaemia, hypertriglyceridemia), cardiovascular diseases, obesity, hemochromatosis, thalassemia, liver injury, alcoholic liver diseases, alcohol dependence and/or anaemia of chronic disease.

The conjugated nucleic acids of the invention or pharmaceutical compositions comprising a conjugated nucleic acid of the invention are preferably for use in the treatment or prevention of a disease or disorder for which it is desirable reduce the expression level of the target gene targeted by the nucleic acid of the invention.

In a further aspect of the invention there is provided a method of delivery of nucleic acids to hepatocytes using the conjugates according to the present invention. The method comprises the steps of contacting the hepatocyte with the compound of the present invention. The method may be used in vitro or in vivo, for diagnostic purposes, therapy or research purposes.

There is provided a method of making a conjugate of the invention the method comprising adding together the components of the conjugate to form the conjugate.

In addition, the invention provides a method of inhibiting (in vitro or in vivo) the expression of a target gene in a mammalian cell, the method comprising contacting the mammalian cell with a conjugate of the invention or a pharmaceutical composition of the invention.

There is also provided a method of inducing RNAi in a subject, the method comprising administering to the subject an effective amount of a conjugate of the invention, or a composition of the invention.

In particular, any one of the above methods may be used in the treatment of liver disease, in particular genetic disease, haemophilia and bleeding disorder, liver fibrosis, non alcoholic steatohepatitis (NASH), non alcoholic fatty liver disease (NAFLD), viral hepatitis, rare diseases (e.g. acromegaly), metabolic diseases (e.g. hypercholesterolemia, dyslipidaemia, hypertriglyceridemia), cardiovascular diseases, obesity, hemochromatosis, thalassemia, liver injury, alcoholic liver diseases, alcohol dependence and/or anaemia of chronic disease in patient in need thereof.

Clauses and Statements

Some aspects of the invention are defined by the following clauses:

1. A conjugate for inhibiting expression of a target gene in a cell, said conjugate comprising a nucleic acid portion and ligand portions, said nucleic acid portion comprising at least one duplex region that comprises at least a portion of a first RNA strand and at least a portion of a second RNA strand that is at least partially complementary to the first strand, wherein said first strand is at least partially complementary to at least a portion of RNA transcribed from said target gene, said ligand portions comprising a linker moiety and a targeting ligand for in vivo targeting of cells and being conjugated exclusively to the 3′ and/or 5′ ends of one or both RNA strands, wherein the 5′ end of the first RNA strand is not conjugated, wherein:
(i) the second RNA strand is conjugated at the 5′ end to the targeting ligand, and wherein (a) the second RNA strand is also conjugated at the 3′ end to the targeting ligand and the 3′ end of the first RNA strand is not conjugated; or (b) the first RNA strand is conjugated at the 3′ end to the targeting ligand and the 3′ end of the second RNA strand is not conjugated; or (c) both the second RNA strand and the first RNA strand are also conjugated at the 3′ ends to the targeting ligand; or
(ii) both the second RNA strand and the first RNA strand are conjugated at the 3′ ends to the targeting ligand and the 5′ end of the second RNA strand is not conjugated.

2. The conjugate according to clause 1 wherein the linker moiety is a serinol-derived linker moiety.

3. The conjugate according to clause 1 or clause 2 wherein the second RNA strand is conjugated at the 5′ end to the targeting ligand, the second RNA strand is also conjugated at the 3′ end to the targeting ligand and the 3′ end of the first RNA strand is not conjugated.

4. The conjugate according to clause 1 or clause 2 wherein the second RNA strand is conjugated at the 5′ end to the targeting ligand, the first RNA strand is conjugated at the 3′ end to the targeting ligand and the 3′ end of the second RNA strand is not conjugated.

5. The conjugate according to clause 1 or clause 2 wherein the second RNA strand is conjugated at the 5′ end to the targeting ligand and both the second RNA strand and the first RNA strand are also conjugated at the 3′ ends to the targeting ligand.

6. The conjugate according to clause 1 or clause 2 wherein both the second RNA strand and the first RNA strand are conjugated at the 3′ ends to the targeting ligand and the 5′ end of the second RNA strand is not conjugated.

7. The conjugate according to any one of clauses 1 to 6 wherein the ligands are monomeric ligands.

8. The conjugate according to any one of clauses 1 to 7 wherein the ligands are selected from GalNAc and galactose moieties, especially GalNAc moieties.

9. The conjugate according to any one of clauses 1 to 8 wherein the conjugated RNA strands are conjugated to a targeting ligand via a linker moiety including a further linker wherein the further linker is or comprises a saturated, unbranched or branched C₁₋₁₅ alkyl chain, wherein optionally one or more carbons (for example 1, 2 or 3 carbons, suitably 1 or 2, in particular 1) is/are replaced by a heteroatom selected from O, N, S(O)ₚ wherein p is 0, 1 or 2, (for example a CH₂ group is replaced with 0, or with NH, or with S, or with SO₂ or a —CH₃ group at the terminus of the chain or on a branch is replaced with OH or with NH₂) wherein said chain is optionally substituted by one or more oxo groups (for example 1 to 3, such as 1 group).

10. The conjugate according to clause 9 wherein the further linker comprises a saturated, unbranched C₁₋₁₅ alkyl chain wherein one or more carbons (for example 1, 2 or 3 carbons, suitably 1 or 2, in particular 1) is/are replaced by an oxygen atom.

11. The conjugate according to clause 10 wherein the further linker comprises a PEG-chain.

12. The conjugate according to clause 9 wherein the further linker comprises a saturated, unbranched C₁₋₁₅ alkyl chain.

13. The conjugate according to clause 12 wherein the further linker comprises a saturated, unbranched C₁₋₆ alkyl chain.

14. The conjugate according to clause 13 wherein the further linker comprises a saturated, unbranched C₄ or C₆ alkyl chain, e.g. a C₄ alkyl chain.

15. The conjugate according to clause 1 or clause 2 wherein the first RNA strand is a compound of formula (IX):

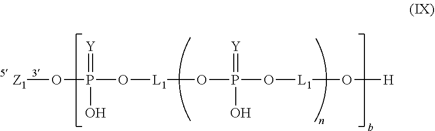

wherein b is 0 or 1; and

the second RNA strand is a compound of formula (X):

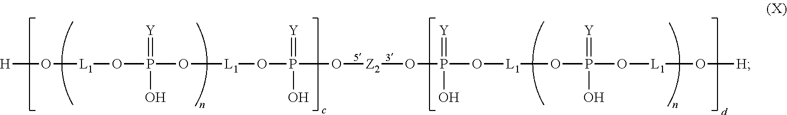

wherein:
c and d are independently 0 or 1;
Z₁ and Z₂ are the RNA portions of the first and second RNA strands respectively;
Y is O or S;
n is 0, 1, 2 or 3; and
L₁ is a linker to which a ligand is attached;

and wherein b+c+d is 2 or 3.

16. The conjugate according to clause 15 wherein the first RNA strand is a compound of formula (XIV):

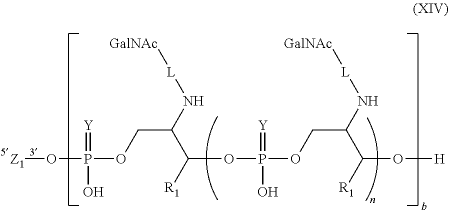

wherein b is 0 or 1; and

the second RNA strand is a compound of formula (XV):

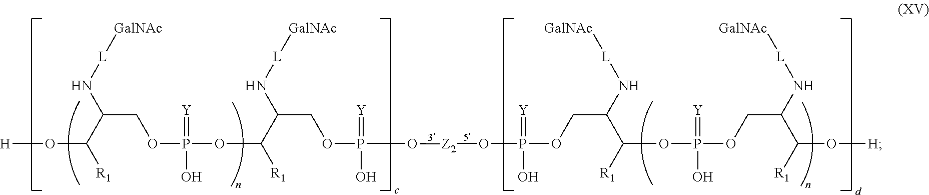

wherein c and d are independently 0 or 1;

wherein:
Z₁ and Z₂ are the RNA portions of the first and second RNA strands respectively;
Y is O or S;
R₁ is H or methyl;
n is 0, 1, 2 or 3; and
L is the same or different in formulae (XIV) and (XV) and is selected from the group consisting of:
   —(CH₂)ᵣC(O)—, wherein r=2-12;
   —(CH₂—CH₂—O)ₛ—CH₂—C(O)—, wherein s=1-5;
   —(CH₂)ₜ—CO—NH—(CH₂)ₜ—NH—C(O)—, wherein t is independently is 1-5;
   —(CH₂)ᵤ—CO—NH—(CH₂)ᵤ—C(O)—, wherein u is independently is 1-5; and
   —(CH₂)ᵥ—NH—C(O)—, wherein v is 2-12; and
wherein the terminal C(O) (if present) is attached to the NH group;

and wherein b+c+d is 2 or 3.

17. The conjugate according to clause 15 wherein the first RNA strand is a compound of formula (XVI):

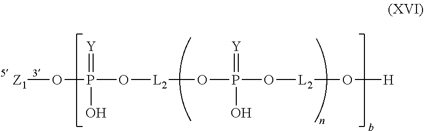

wherein b is 0 or 1; and

the second RNA strand is a compound of formula (XVII):

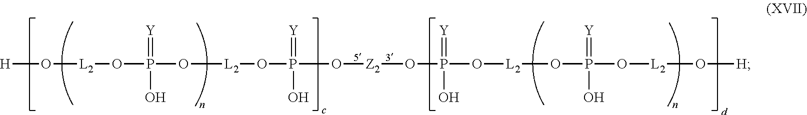

wherein:
c and d are independently 0 or 1;
Z₁ and Z₂ are the RNA portions of the first and second RNA strands respectively;
Y is O or S;
n is 0, 1, 2 or 3; and
L₂ is the same or different in formulae (XVI) and (XVII) and is the same or different in moieties bracketed by b, c and d, and is selected from the group consisting of:

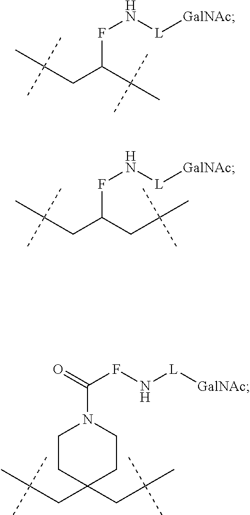

and optionally

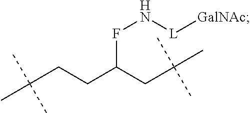

and optionally

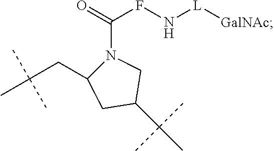

or
n is 0 and L₂ is:

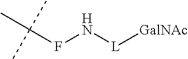

and the terminal OH group is absent such that the following moiety is formed:

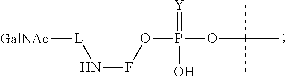

wherein
F is saturated branched or unbranched (such as unbranched) C₁₋₈alkyl (e.g. C₁₋₆alkyl) chain wherein one of the carbon atoms is optionally replaced with an oxygen atom provided that said oxygen atom is separated from another heteroatom (e.g. an O or N atom) by at least 2 carbon atoms;
L is the same or different in formulae (XVI) and (XVII) and is selected from the group consisting of:
   —(CH₂)ᵣC(O)—, wherein r=2-12;
   —(CH₂—CH₂—O)ₛ—CH₂—C(O)—, wherein s=1-5;
   —(CH₂)ₜ—CO—NH—(CH₂)ₜ—NH—C(O)—, wherein t is independently is 1-5;
   —(CH₂)ᵤ—CO—NH—(CH₂)ᵤ—C(O)—, wherein u is independently is 1-5; and
   —(CH₂)ᵥ—NH—C(O)—, wherein v is 2-12; and
wherein the terminal C(O) (if present) is attached to the NH group;

and wherein b+c+d is 2 or 3.

18. The conjugate according to any one of clauses 15-17 wherein b is 0, c is 1 and d is 1.

19. The conjugate according to any one of clauses 15-17 wherein b is 1, c is 0 and d is 1.

20. The conjugate according to any one of clauses 15-17 wherein b is 1, c is 1 and d is 0.

21. The conjugate according to any one of clauses 15-17 wherein b is 1, c is 1 and d is 1.

22. The conjugate according to any one of clauses 15-21 wherein Y is O.

23. The conjugate according to any one of clauses 15-21 wherein Y is S.

24. The conjugate according to any one of clauses 16 or 18-23 wherein R₁ is H.

25. The conjugate according to any one of clauses 16 or 18-23 wherein R₁ is methyl.

26. The conjugate according to any one of clauses 15-25 wherein n is 0.

27. The conjugate according to any one of clauses 16-25 wherein L is —(CH₂)ᵣ—C(O)—, wherein r=2-12.

28. The conjugate according to clause 27 wherein r=2-6.

29. The conjugate according to clause 28 wherein r=4 or 6 e.g. 4.

30. The conjugate according to any one of clauses 17-29 wherein L₂ is:

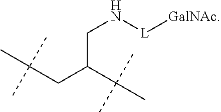

31. The conjugate according to any one of clauses 17-29 wherein L₂ is:

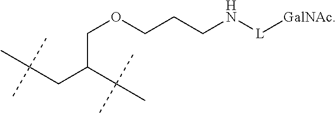

32. The conjugate according to any one of clauses 17-29 wherein L₂ is:

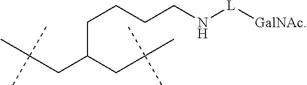

33. The conjugate according to any one of clauses 17-29 wherein L₂ is:

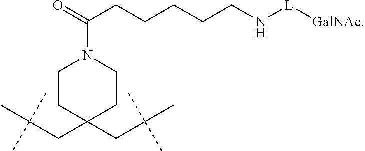

34. The conjugate according to any one of clauses 17-29 wherein n is 0 and L₂ is:

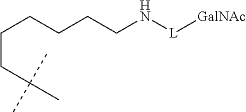

and the terminal OH group is absent such that the following moiety is formed:

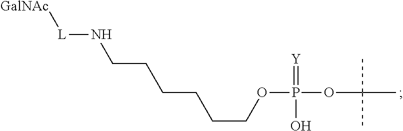

wherein Y is as defined in clause 17.

35. The conjugate according to any one of clause 1-34 wherein the targeted cells are hepatocytes.

36. The conjugate according to any one of clauses 1-35 wherein the nucleic acid portion comprises or consists of two RNA strands of 15-30 based-paired ribonucleotides, suitably 19-25 e.g. 19-23 based-paired ribonucleotides.

37. The conjugate according to any one of clauses 1-36 wherein the first RNA strand has one to six (e.g. one to three) phosphorothioate internucleotide linkages at each end.

38. The conjugate according to any one of clauses 1-37 wherein the second RNA strand has one to six (e.g. one to three) phosphorothioate internucleotide linkages at each end.

39. A method of making the conjugate, as described in any one of clauses 1-38, the method comprising adding together the components of the conjugate to form the conjugate.

40. A pharmaceutical composition comprising the conjugate according to any one of clauses 1-38 together with a pharmaceutically acceptable diluent or carrier.

41. The conjugate according to any one of clauses 1-38 or the pharmaceutical composition according to clause 40 for use in medicine.

42. The conjugate or composition, for use as described in clause 41, wherein the use for treating liver disease, genetic disease, haemophilia and bleeding disorder, liver fibrosis, non alcoholic steatohepatitis (NASH), non alcoholic fatty liver disease (NAFLD), viral hepatitis, rare diseases (e.g. acromegaly), metabolic diseases (e.g. hypercholesterolemia, dyslipidaemia, hypertriglyceridemia), cardiovascular diseases, obesity, hemochromatosis, thalassemia, liver injury, alcoholic liver diseases, alcohol dependence and/or anaemia of chronic disease.

43. A method of inhibiting (in vitro or in vivo) the expression of a target gene in a mammalian cell, the method comprising contacting the mammalian cell with the conjugate as defined in any one of clauses 1 to 38, or a composition as described in clause 40.

44. A method of inducing RNAi in a subject, the method comprising administering to the subject an effective amount of the conjugate as described in any one of clauses 1 to 38, or a composition as described in clause 40.

45. A method as described in clause 43 or clause 44 for use in the treatment of liver disease, genetic disease, haemophilia and bleeding disorder, liver fibrosis, non-alcoholic steatohepatitis (NASH), non-alcoholic fatty liver disease (NAFLD), viral hepatitis, rare diseases (e.g. acromegaly), metabolic diseases (e.g. hypercholesterolemia, dyslipidaemia, hypertriglyceridemia), cardiovascular diseases, obesity, hemochromatosis, thalassemia, liver injury, alcoholic liver diseases, alcohol dependence and/or anaemia of chronic disease in patient in need thereof.

Some aspects of the invention are defined by the following statements:

1. A conjugate for inhibiting expression of a target gene in a cell, said conjugate comprising a nucleic acid portion and ligand portions, said nucleic acid portion comprising at least one duplex region that comprises at least a portion of a first RNA strand and at least a portion of a second RNA strand that is at least partially complementary to the first strand, wherein said first strand is at least partially complementary to at least a portion of RNA transcribed from said target gene, said ligand portions comprising a serinol-derived linker moiety and a targeting ligand for in vivo targeting of cells and being conjugated exclusively to the 3′ and/or 5′ ends of one or both RNA strands, wherein the 5′ end of the first RNA strand is not conjugated, wherein:
(i) the second RNA strand is conjugated at the 5′ end to the targeting ligand, and wherein (a) the second RNA strand is also conjugated at the 3′ end to the targeting ligand and the 3′ end of the first RNA strand is not conjugated; or (b) the first RNA strand is conjugated at the 3′ end to the targeting ligand and the 3′end of the second RNA strand is not conjugated; or (c) both the second RNA strand and the first RNA strand are also conjugated at the 3′ ends to the targeting ligand; or
(ii) both the second RNA strand and the first RNA strand are conjugated at the 3′ ends to the targeting ligand and the 5′ end of the second RNA strand is not conjugated;

wherein said first strand includes modified nucleotides at a plurality of positions, and wherein the nucleotides at positions 2 and 14 from the 5′ end of the first strand are not modified with a 2′-OMe modification

or

a conjugate for inhibiting expression of a target gene in a cell, said conjugate comprising a nucleic acid portion and ligand portions, said nucleic acid portion comprising at least one duplex region that comprises at least a portion of a first RNA strand and at least a portion of a second RNA strand that is at least partially complementary to the first strand, wherein said first strand is at least partially complementary to at least a portion of RNA transcribed from said target gene, said ligand portions comprising a linker moiety and a targeting ligand for in vivo targeting of cells and being conjugated exclusively to the 3′ and/or 5′ ends of one or both RNA strands, wherein the 5′ end of the first RNA strand is not conjugated, wherein:
(i) the second RNA strand is conjugated at the 5′ end to the targeting ligand, and wherein (a) the second RNA strand is also conjugated at the 3′ end to the targeting ligand and the 3′ end of the first RNA strand is not conjugated; or (b) the first RNA strand is conjugated at the 3′ end to the targeting ligand and the 3′end of the second RNA strand is not conjugated; or (c) both the second RNA strand and the first RNA strand are also conjugated at the 3′ ends to the targeting ligand; or
(ii) both the second RNA strand and the first RNA strand are conjugated at the 3′ ends to the 20 targeting ligand and the 5′ end of the second RNA strand is not conjugated

wherein the first strand of the nucleic acid includes modified nucleotides at a plurality of positions, and wherein the nucleotides at positions 2 and 14 from the 5′ end of the first strand are not modified with a 2′-OMe modification.

2. A nucleic acid, particularly a conjugate, of statement 1 wherein the nucleotides at positions 2 and 14 from the 5′ end of the first strand are modified.

3 A nucleic acid or more specifically a conjugate according to statement 2, wherein the nucleotides at positions 2 and 14 from the 5′ end of the first strand are not modified with a 2′-OMe modification, and the nucleotide on the second strand which corresponds to position 13 of the first strand is not modified with a 2′-OMe modification.

4 A nucleic acid, particularly a conjugate, according to statement 2, wherein the nucleotides at positions 2 and 14 from the 5′end of the first strand are not modified with a 2′-OMe modification, and the nucleotide on the second strand which corresponds to position 11 of the first strand is not modified with a 2′-OMe modification.

5 A nucleic acid, particularly a conjugate, according to statement 2-4 wherein the nucleotides at positions 2 and 14 from the 5′ end of the first strand are not modified with a 2′-OMe modification, and the nucleotides on the second strand which correspond to position 11 and 13 of the first strand are not modified with a 2′-OMe modification.

6 A nucleic acid, particularly a conjugate, of any preceding statement wherein the nucleotides on the second strand corresponding to positions 11 and/or 13 from the 5′ end of the first strand are modified.

7 A nucleic acid, particularly a conjugate, according to statements 2-6, wherein the nucleotides at positions 2 and 14 from the 5′ end of the first strand are not modified with a 2′-OMe modification, and the nucleotides on the second strand which correspond to position 11, or 13, or 11 and 13, or 11-13 of the first strand are modified with a 2′-F modification.

8 A nucleic acid, particularly a conjugate, according to any one of statements 2-7, wherein the nucleotides at positions 2 and 14 from the 5′ end of the first strand are modified with a 2′-F modification, and the nucleotides on the second strand which correspond to position 11, or 13, or 11 and 13, or 11-13 of the first strand are not modified with a 2′-OMe modification.

9 A nucleic acid, particularly a conjugate, according to any of statements 2-8 wherein the nucleotides at positions 2 and 14 from the 5′ end of the first strand are modified with a 2′-F modification, and the nucleotides on the second strand which correspond to position 11, or 13, or 11 and 13, or 11-13 of the first strand are modified with a 2′-F modification.

10 A nucleic acid, particularly a conjugate, according to any preceding statements wherein greater than 50% of the nucleotides of the first and/or second strand comprise a 2′-OMe modification, such as greater than 55%, 60%, 65%, 70%, 75%, 80%, or 85%, or more, of the first and/or second strand comprise a 2′-OMe modification, preferably measured as a percentage of the total nucleotides of both the first and second strands.

11 A nucleic acid, particularly a conjugate, according to any preceding statement comprising no more than 20%, (such as no more than 15% or no more than 10%) of 2′-F modifications on the first and/or second strand, as a percentage of the total nucleotides of both strands.

12 A nucleic acid, particularly a conjugate, according to any preceding statement wherein the terminal nucleotide at the 3′ end of at least one of the first strand and the second strand is an inverted nucleotide and is attached to the adjacent nucleotide via the 3′ carbon of the terminal nucleotide and the 3′ carbon of the adjacent nucleotide and/or the terminal nucleotide at the 5′ end of at least one of the first strand and the second strand is an inverted nucleotide and is attached to the adjacent nucleotide via the 5′ carbon of the terminal nucleotide and the 5′ carbon of the adjacent nucleotide, or wherein the nucleic acid comprises a phosphorodithioate linkage.

13. A composition comprising a conjugated nucleic acid of any of statements 1-12 and a physiologically acceptable excipient.

14. A nucleic acid or conjugated nucleic acid according to any of statements 1 to 12 or a composition of statement 13 for use in the treatment of a disease or disorder.

15. Use of a nucleic acid or conjugated nucleic acid according to any of statements 1 to 12 or a composition of statement 13 in the manufacture of a medicament for treating a disease or disorder.

16. A method of treating a disease or disorder comprising administration of a composition comprising a nucleic acid or conjugated nucleic acid according to any of statements 1 to 13 to an individual in need of treatment.

17. A conjugate for inhibiting expression of a TMPRSS6 gene in a cell, said conjugate comprising a nucleic acid portion and ligand portions, said nucleic acid portion comprising at least one duplex region that comprises at least a portion of a first RNA strand and at least a portion of a second RNA strand that is at least partially complementary to the first strand, wherein said first strand is at least partially complementary to at least a portion of RNA transcribed from said TMPRSS6 gene, said ligand portions comprising a linker moiety, preferably a serinol-derived linker moiety, and a targeting ligand for in vivo targeting of cells and being conjugated exclusively to the 3′ and/or 5′ ends of one or both RNA strands, wherein the 5′ end of the first RNA strand is not conjugated, wherein the second RNA strand is conjugated at the 5′ end to the targeting ligand, and wherein the second RNA strand is also conjugated at the 3′ end to the targeting ligand and the 3′ end of the first RNA strand is not conjugated; and wherein said first strand includes modified nucleotides at a plurality of positions, and wherein the nucleotides at positions 2 and 14 from the 5′ end of the first strand are not modified with a 2′-OMe modification and the second strand positions opposite first strand positions 11, 12, and 13 (corresponding to second strand positions 7, 8, and 9 from the 5′end in a 19-mer) are not modified with 2′-OMe modification.

18. A conjugate according to statement 17, wherein the first RNA strand may comprise the nucleotide sequence of X0371A and/or the second RNA strand may comprise the nucleotide sequence of X0371B:

X0371A
mA (ps) fA (ps) mC fC mA fG mA fA mG fA mA fG mC
fA mG fG mU (ps) fG (ps) mA
X0371B
Ser(GN) (ps) mU (ps) mC (ps) mA mC mC mU fG fC
fU mU mC mU mU mC mU mG mG (ps) mU (ps) mU (ps)
Ser(GN)

19. A conjugate for inhibiting expression of a target gene in a cell, said conjugate comprising a nucleic acid portion and ligand portions, said nucleic acid portion comprising at least one duplex region that comprises at least a portion of a first RNA strand and at least a portion of a second RNA strand that is at least partially complementary to the first strand, wherein said first strand is at least partially complementary to at least a portion of RNA transcribed from said target gene, said ligand portions comprising a linker moiety and a targeting ligand for in vivo targeting of cells and being conjugated exclusively to the 3′ and/or 5′ ends of one or both RNA strands, wherein the 5′ end of the first RNA strand is not conjugated, wherein:
(i) the second RNA strand is conjugated at the 5′ end to the targeting ligand, and wherein (a) the second RNA strand is also conjugated at the 3′ end to the targeting ligand and the 3′ end of the first RNA strand is not conjugated; or (b) the first RNA strand is conjugated at the 3′ end to the targeting ligand and the 3′ end of the second RNA strand is not conjugated; or (c) both the second RNA strand and the first RNA strand are also conjugated at the 3′ ends to the targeting ligand; or
(ii) both the second RNA strand and the first RNA strand are conjugated at the 3′ ends to the targeting ligand and the 5′ end of the second RNA strand is not conjugated, and wherein the first strand of the nucleic acid includes modified nucleotides at a plurality of positions, and wherein the nucleotides at positions 2 and 14 from the 5′ end of the first strand are not modified with a 2′-OMe modification.

20. A conjugate according to statement 19, wherein the linker moiety is a serinol-derived linker moiety or one of the other linker types described herein.

### Advantages

The conjugates of the invention, in at least some embodiments, are expected to have one or more of the following advantageous properties
good gene knock-down potency in an RNAi setting;
good duration of action;
good stability;
good targeting of cells by the conjugated ligand;
resistance to various nucleases;
alleviation of immune response induction;
improved circulation and tissue uptake;
targeting cells by the conjugated ligand;
uptake by cells without additional delivery means;
activation of RNAi-mediated target gene down-regulation; and
ease of manufacture.

### EXAMPLES

### Abbreviations

% FLP percentage full length product
° C. degrees centigrade
1H NMR proton nuclear magnetic resonance
A angstrom(s)
Ac acetyl/acetic
AEX-HPLC Anion Exchange High Pressure Liquid Chromatography
Cap Capping
CEP cyanoethyl phosphoramidite
CPG controlled pore glass
Da dalton(s)
DCM Dichloromethane
DEA Diethylamine
DIEA Diisoethylamine
DIPEA Diisopropylethylamine
DMAP 4-dimethylaminopyridine
DMSO Dimethylsulphoxide
DMT® Dimethoxytrityl
EDTA ethylenediaminetetraacetic acid
Eq equivalent(s)
ESI- electrospray ionization
Et Ethyl
G gram(s)
H hour(s)
HBTU 2-(1H-benzotriazol-1-yl)-1,1,3,3-tetramethyluronium hexafluorophosphate
HPLC high performance liquid chromatography
iPr iso-propyl
Kg kilogram(s)
Lcaa long chain amino alkyl
LCMS liquid chromatography mass spectrometry
M Molar
Me Methyl
Min minute(s)
mL millilitre(s)
MPEG methylated polyethylene glycol
MW molecular weight
Nm Nanometer
NMI N-methylimidazole
OD optical density (absorption)
OX Oxidization
PEG Polyethyleneglycol
PNA peptide nucleic acid
RT room temperature
s.c. Subcutaneous
TFA trifluoroacetic acid
THE Tetrahydrofuran
TLC thin layer chromatography
TMS trimethylsilyl/trimethylsilane
TTR Transthyretin
U Micro
UV Ultraviolet
v/v volume/volume

General Methods

In Vitro Experiments

Primary murine hepatocytes (Thermo Scientific: GIBCO Lot: #MC798) were thawed and cryo-preservation medium exchanged for Williams E medium supplemented with 5% FBS, 1 pM dexamethasone, 2 mM GlutaMax, 1% PenStrep, 4 mg/ml human recombinant insulin, 15 mM Hepes. Cell density was adjusted to 250000 cells per 1 ml. 100 μl per well of this cell suspension were seeded into collagen pre-coated 96 well plates. The test article was prediluted in the same medium (5 times concentrated) for each concentration and 25 μl of this prediluted siRNA or medium only were added to the cells. Cells were cultured in at 37° C. and 5% CO₂. 24 h post treatment the supernatant was discarded, and cells were washed in cold PBS and 250 μl RNA-Lysis Buffer S (Stratec) was added. Following 15 min incubation at room temperature plates were storage at −80° C. until RNA isolation according to the manufacturers protocol.

TaqMan Analysis

For mTTR &PTEN MultiPlex TaqMan analysis 10 μl isolated RNA for each treatment group were mixed with 10 μl PCR mastermix (TAKYON low Rox) containing 600 nM mTTR-primer, 400 nM ApoB-primer and 200 nM of each probe as well as 0.5 units Euroscript IIRT polymerase with 0.2 units RNAse inhibitor. TaqMan analysis was performed in 384-well plate with a10 min RT step at 48° C., 3 min initial denaturation at 95° C. and 40 cycles of 95° C. for 10 sec and 60° C. for 1 min.

For TMPRSS6 &ApoB MultiPlex TaqMan analysis 10 μl isolated RNA for each treatment group were mixed with 10 μl PCR mastermix (TAKYON low Rox) containing 800 nM TMPRSS6 primer, 100 nM ApoB primer and 200 nM of either probe as well as 0.5 units Euroscript IIRT polymerase with 0.2 units RNAse inhibitor. TaqMan analysis was performed in 384-well plate with a10 min RT step at 48° C., 3 min initial denaturation at 95° C. and 40 cycles of 95° C. for 10 sec and 60° C. for 1 min.

Primers

| | | |
|---|---|---|
| TTR | fw | TGGACACCAAATCGTACTGGAA |
| | rev | CAGAGTCGTTGGCTGTGAAAAC |
| | probe | BHQ1-ACTTGGCATTTCCCCGTTCCATGAATT- |
| | | FAM |
| PTEN | fw | CACCGCCAAATTTAACTGCAGA |
| | rev | AAGGGTTTGATAAGTTCTAGCTGT |
| | probe | BHQ1-TGCACAGTATCCTTTTGAAGACCATAACC |
| | | CA-YY |
| ALDH2 | fw | GGCAAGCCTTATGTCATCTCGT |
| | rev | GGAATGGTTTTCCCATGGTACTT |
| | probe | BHQ1-TGAAATGTCTCCGCTATTACGCTGGCTG- |
| | | FAM |
| TMPRSS6 | fw | CGGCACCTACCTTCCACTCTT |
| | rev | TCGGTGGTGGGCATCCT |
| | probe | BHQ1-CCGAGATGTTTCCAGCTCCCCTGTTCTA- |
| | | FAM |
| LPA | fw | GTGTCCTCGCAACGTCCA |
| | rev | GACCCCGGGGCTTTG |
| | probe | BHQ1-TGGCTGTTTCTGAACAAGCACCAATGG- |
| | | FAM |
| ACTB | fw | GCATGGGTCAGAAGGATTCCTAT |
| | rev | TGTAGAAGGTGTGGTGCCAGATT |
| | probe | BHQ1-TCGAGCACGGCATCGTCACCAA-YY |

FAM = 6-Carboxyfluorescein; BHQ = Black Hole Quencher 1; YY = Yakima Yellow

In Vivo Experiments

To compare in vivo potency of different siRNA conjugates 1 mg/kg siRNA dissolved in PBS was administered sub cutaneous in the scapular region of c57BL/6 mice. Cohorts of of n=6 for were treated with siRNA targeting ALDH2 or TMPRSS6 at day 1 and sacrificed at selected times points post treatment. Liver samples were snap frozen in liquid nitrogen and stored at −80° C. until extraction RNA with InviTrap Spin Tissue RNA Mini Kit (stratec) according to the manufacturers manual. Following, transcript level of ALDH2, TMPRSS6 and PTEN were quantified as described above.

Tritosome Stability Assay

To probe for RNAase stability in the endosomal/lysosomal compartment of hepatic cells in vitro siRNA was incubated for 0 h, 4 h, 24 h or 72 h in Sprague Dawley Rat Liver Tritosomes (Tebu-Bio, CatN.: R0610.LT, lot: 1610405, pH: 7.4, 2.827 Units/ml). To mimic the acidified environment the Tritosomes were mixed 1:10 with low pH buffer (1.5M acetic acid, 1.5M sodium acetate pH 4.75). 30 μl of this acidified Tritosomes. Following 10 μl siRNA (20 μM) were mixed with and incubated for the indicated times at 37° C. Following incubation RNA was isolated with the Clarity OTX Starter Kit-Cartriges (Phenomenex CatNo: KSO-8494) according to the manufactures protocol for biological fluids. Lyophilized RNA was reconstituted in 30 μl H₂O, mixed with 4× loading buffer and 5 μl were loaded to a 20% TBE-polyacrylamide gel electrophoresis (PAGE) for separation qualitative semi-quantitative analysis. PAGE was run at 120 V for 2 h and RNA visualized by Ethidum-bromide staining with subsequent digital imaging with a Biorad Imaging system.

General Synthesis Schemes

Example compounds can be synthesised according to methods described below and known to the person skilled in the art. Whilst the schemes illustrate the synthesis of particular conjugates, it will be understood that other claimed conjugates may be prepared by analogous methods. Assembly of the oligonucleotide chain and linker building blocks may, for example, be performed by solid phase synthesis applying phosphoramidite methodology. Solid phase synthesis may start from a base or modified building block loaded Icaa CPG. Phosphoramidite synthesis coupling cycle consists of 1) DMT-removal, 2) chain elongation using the required DMT-masked phosphoramidite and an activator, which may be benzylthiotetrazole (BTT), 3) capping of non-elongated oligonucleotide chains, followed by oxidation of the P(Ill) to P(V) either by Iodine (if phosphodiester linkage is desired) or EDITH (if phosphorothioate linkage is desired) and again capping (Cap/Ox/Cap or Cap/Thio/Cap). GalNAc conjugation may be achieved by peptide bond formation of a GalNAc-carboxylic acid building block to the prior assembled and purified oligonucleotide having the necessary number of amino modified linker building blocks attached. The necessary building blocks are either commercially available or synthesis is described below.

All final single stranded products were analysed by AEX-HPLC to prove their purity. Purity is given in % FLP (% full length product) which is the percentage of the UV-area under the assigned product signal in the UV-trace of the AEX-HPLC analysis of the final product. Identity of the respective single stranded products was proved by LC-MS analysis.

Synthesis of Synthons

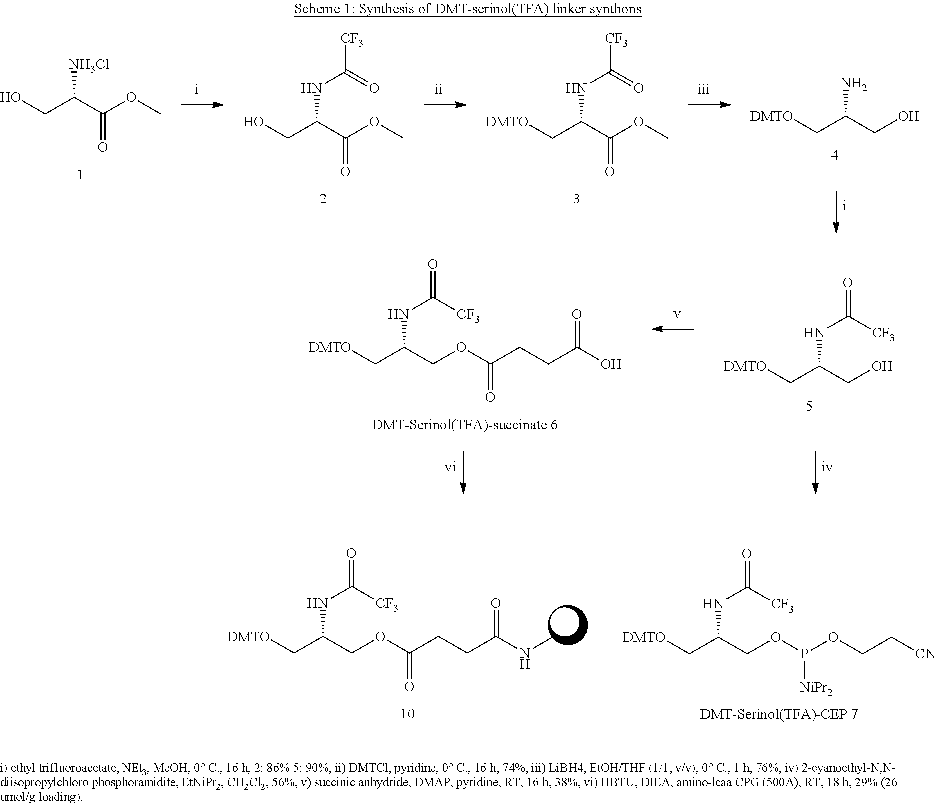

(S)-DMT-Serinol(TFA)-phosphoramidite 7 can be synthesised from (L)-serine methyl ester derivative 1 according to literature published methods (Hoevelmann et al. Chem. Sci., 2016,7, 128-135).

(S)-DMT-Serinol(TFA)-succinate 6 can be made by conversion of intermediate 5 with succinic anhydride in presence of a catalyst such as DMAP.

Loading of 6 to a solid support such as a controlled pore glass (CPG) support may be achieved 15 by peptide bond formation to a solid support such as an amino modified native CPG support (500A) using a coupling reagent such as HBTU. The (S)-DMT-Serinol(TFA)-succinate 6 and a coupling reagent such as HBTU is dissolved in a solvent such as CH₃CN. A base, such as diisopropylethylamine, is added to the solution, and the reaction mixture is stirred for 2 min. A solid support such as a native amino-Icaa-CPG support (500 A, 3 g, amine content: 136 umol/g) is added to the reaction mixture and a suspension forms. The suspension is gently shaken at room temperature on a wrist-action shaker for 16h then filtered, and washed with solvent such as DCM and EtOH. The support is dried under vacuum for 2 h. The unreacted amines on the support can be capped by stirring with acetic anhydride/lutidine/N-methylimidazole at room temperature. Washing of the support may be repeated as above. The solid support is dried under vacuum to yield solid support 10.

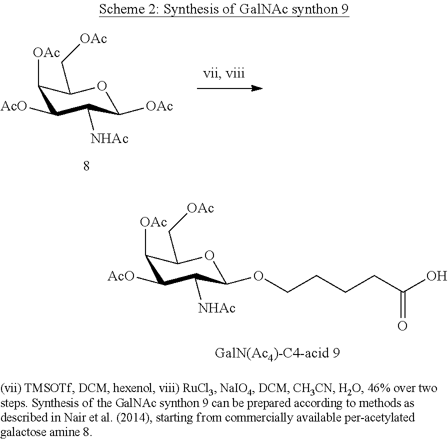

Synthesis of Single Stranded Serinol-Derived GalNAc Conjugates

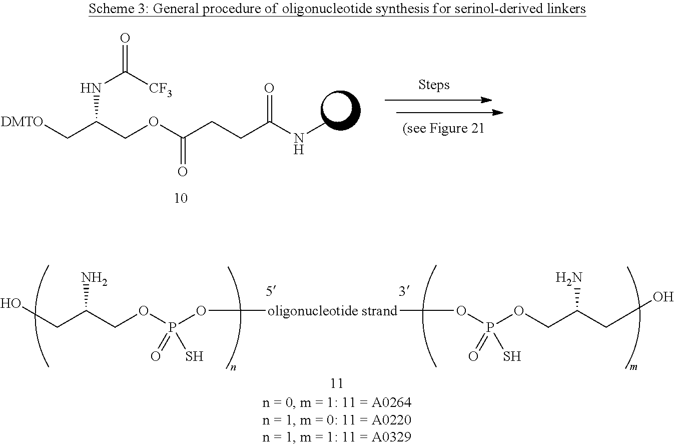

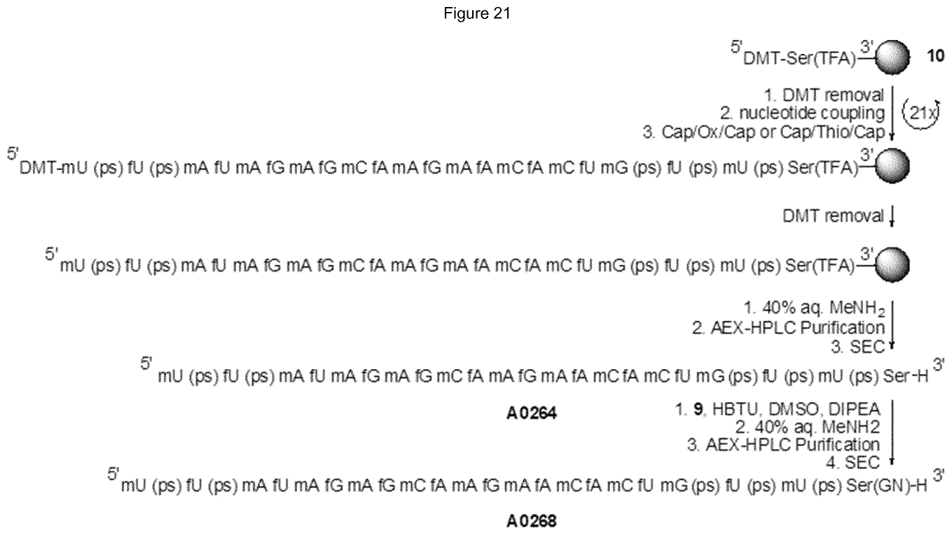

Oligonucleotide Synthesis of 3′ Mono-GalNAc Conjugated Oligonucleotides (Such as Compound A0264) is outlined in FIG. 21 and summarised in Scheme 3. Synthesis is commenced using (S)-DMT-Serinol(TFA)-succinate-Icaa-CPG 10 as in example compound A0264. In case additional serinol building blocks are needed the (S)-DMT-serinol(TFA) amidite (7) is used in the appropriate solid phase synthesis cycle. For example, to make compound A0329, the chain assembly is finished with an additional serinol amidite coupling after the base sequence is fully assembled. Further, oligonucleotide synthesis of 5′ mono-GalNAc conjugated oligonucleotides may be commenced from a solid support loaded with the appropriate nucleoside of its respected sequence. In example compound A0220 this may be 2-′fA. The oligonucleotide chain is assembled according to its sequence and as appropriate, the building block (S)-DMT-serinol(TFA)-amidite (7) is used. Upon completion of chain elongation, the protective DMT group of the last coupled amidite building block is removed, as in step 1) of the phosphoramidite synthesis cycle.

Upon completion of the last synthesizer step, the single strands can be cleaved off the solid support by treatment with an amine such as 40% aq. methylamine treatment. Any remaining protecting groups are also removed in this step and methylamine treatment also liberates the serinol amino function. The crude products were then purified each by AEX-HPLC and SEC to yield the precursor oligonucleotide for further GalNAc conjugation.

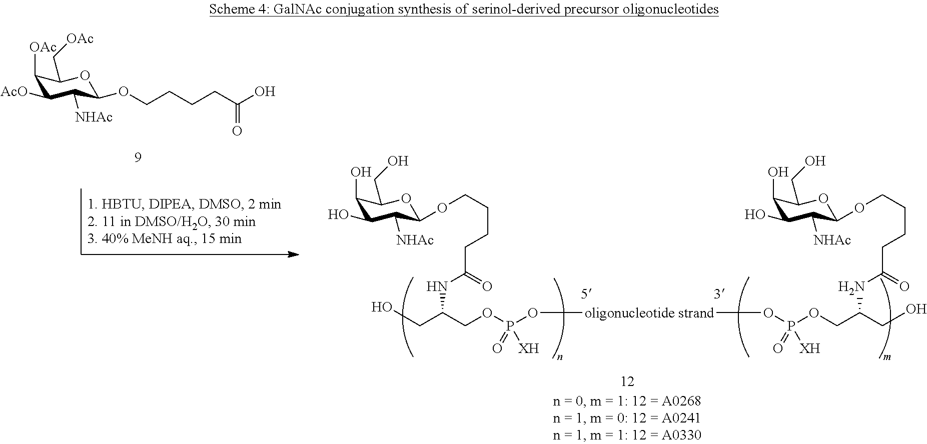

Post solid phase synthesis GalNAc-conjugation was achieved by pre-activation of the GalN(Ac4)-C4-acid (9) by a peptide coupling reagent such as HBTU. The pre-activated acid 9 was then reacted with the amino-groups in 11 (e.g. A0264) to form the intermediate GalN(Ac4)-conjugates. The acetyl groups protecting the hydroxyl groups in the GalNAc-moieties were cleaved off by methylamine treatment to yield the desired example compounds 12 (e.g. A0268), which were further purified by AEX-HPLC and SEC.

Synthesis of Single Stranded Non-Serinol-Derived GalNAc Conjugates

Amino modified building blocks other than serinol are commercially available from various suppliers and can be used instead of serinol to provide reactive amino-groups that allow for GalNAc conjugation. For example the commercially available building blocks shown in Table 2 below can be used to provide non-serinol-derived amino modified precursor oligonucleotides 14 (Scheme 5A) by using amino-modifier loaded CPG such as 10-1 to 10-3 followed by sequence assembly as described above and finally coupling of amino-modifier phosphoramidites such as 13-1, 13-2 or 13-4.

For example, to make 14 (A0653) GlyC3Am-CPG (10-2) was used in combination with GlyC3Am-Amidite 13-2. Structurally differing modifiers can be used to make 14, for example for A0651 C7Am-CPG was used in combination with C6Am-Amidite as second amino modification. In a similar fashion commercially available amino-modifier loaded CPG 10-5 and amino-modified phosphoramidite 13-5 can be used to synthesise amino-modified precursor molecules 14 (A0655).

TABLE 2
Commercially available building blocks

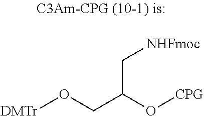

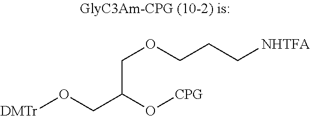

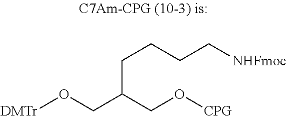

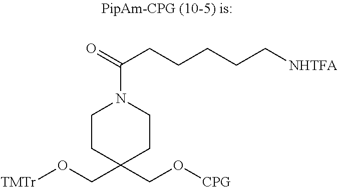

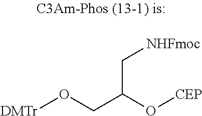

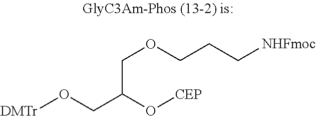

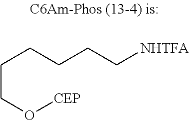

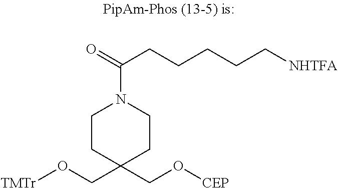

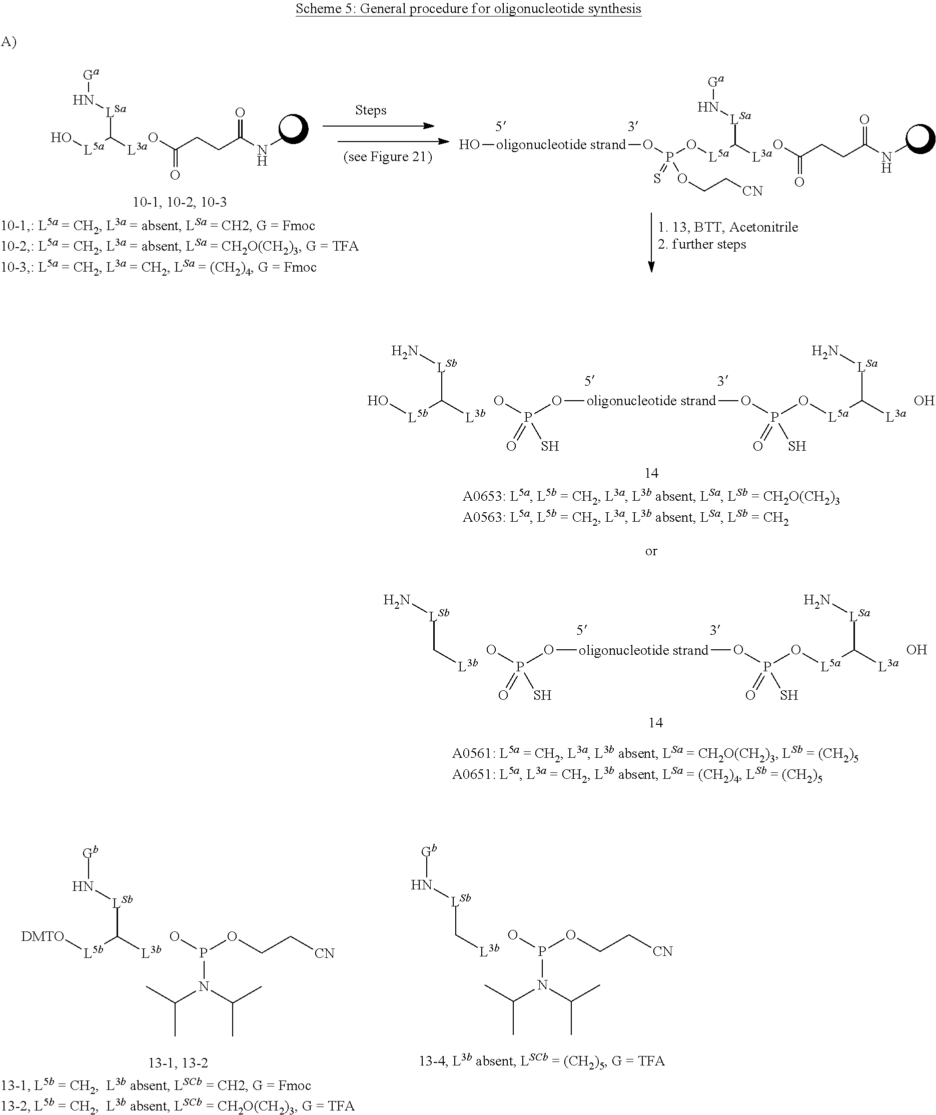

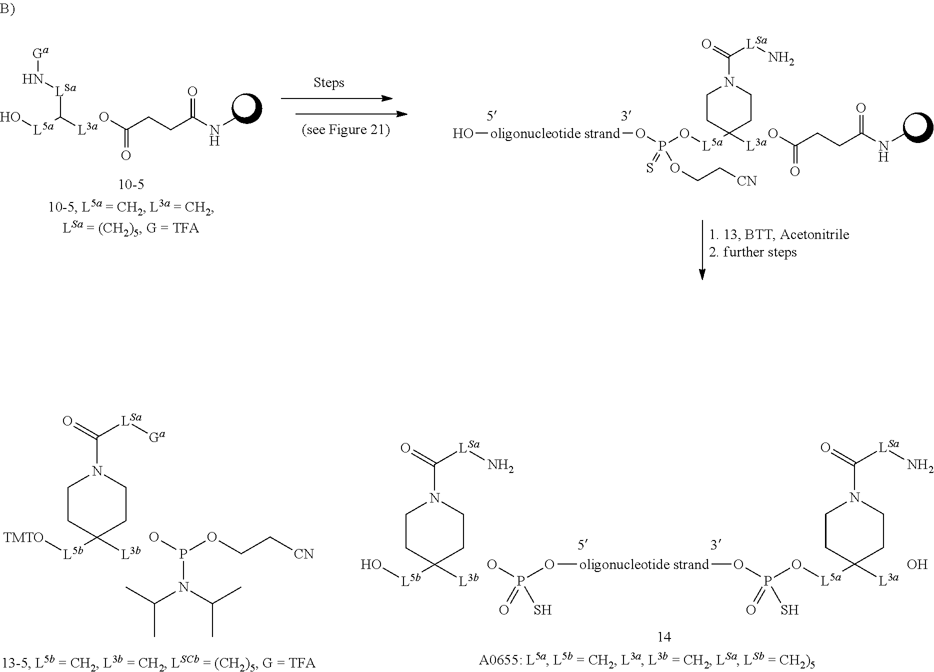

The resulting precursor oligonucleotides 14 can then be conjugated with GalN(Ac4)-C4-acid (9) to yield the desired example compounds 15 (Scheme 6).

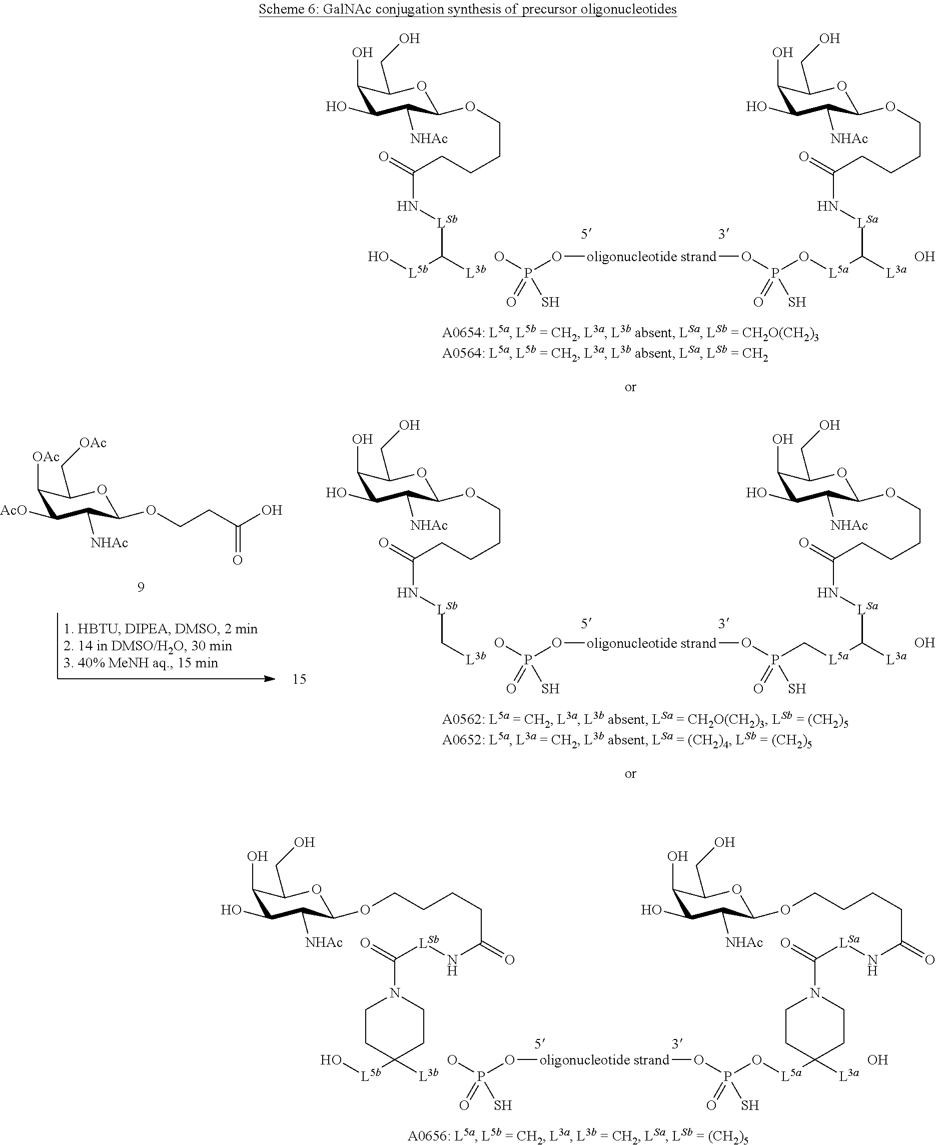

Synthesis of Single Stranded Tri-Antennary GalNAc Conjugates in Reference Conjugates 3-4

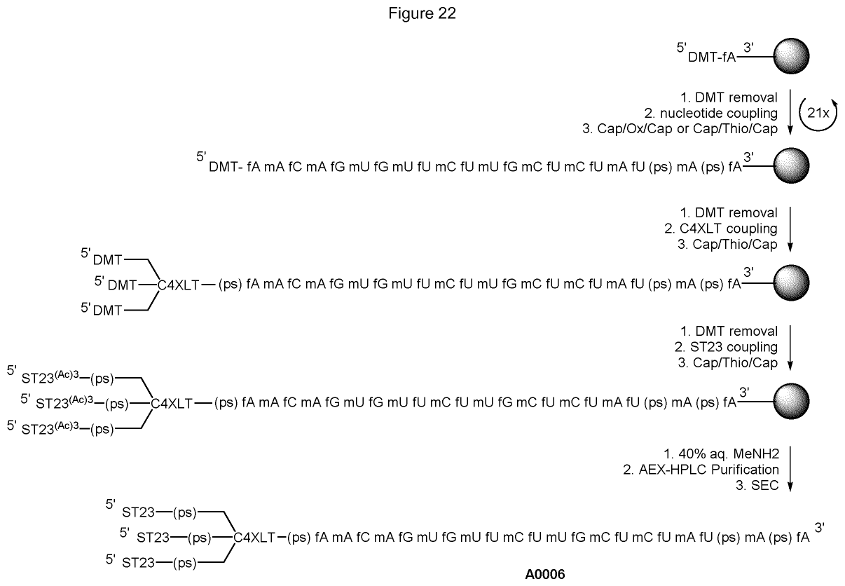

Oligonucleotides synthesis of tri-antennary GalNAc-cluster conjugated siRNA is outlined in FIG. 22. Oligonucleotide chain assembly is commenced using base loaded support e.g. 5′DMT-2′-FdA(bz)-succinate-Icaa-CPG as in example compound A0006. Phosphoramidite synthesis coupling cycle consisting of 1) DMT-removal, 2) chain elongation using the required DMT-masked phosphoramidite, 3) capping of non-elongated oligonucleotide chains, followed by oxidation of the P(Ill) to P(V) either by Iodine or EDITH (if phosphorothioate linkage is desired) and again capping (Cap/Ox/Cap or Cap/Thio/Cap) is repeated until full length of the product is reached. For the on column conjugation of a trivalent tri-antennary GalNAc cluster the same synthesis cycle was applied with using the necessary trivalent branching amidite C4XLT-phos followed by another round of the synthesis cycle using the GalNAc amidite ST23-phos. Upon completion of this last synthesizer step, the oligonucleotide was cleaved from the solid support and additional protecting groups may be removed by methylamine treatment. The crude products were then purified each by AEX-HPLC and SEC.

General Procedure of Double Strand Formation

In order to obtain the double stranded conjugates, individual single strands are dissolved in a concentration of 60 OD/mL in H₂O. Both individual oligonucleotide solutions can be added together to a reaction vessel. For reaction monitoring a titration can be performed. The first strand is added in 25% excess over the second strand as determined by UV-absorption at 260 nm. The reaction mixture is heated e.g. to 80° C. for 5 min and then slowly cooled to RT. Double strand formation may be monitored by ion pairing reverse phase HPLC. From the UV-area of the residual single strand the needed amount of the second strand can be calculated and added to the reaction mixture. The reaction is heated e.g. to 80° C. again and slowly cooled to RT. This procedure can be repeated until less than 10% of residual single strand is detected.

The above process (including Schemes 1-6 and FIGS. 21 and 22) may be easily adapted to replace GalNac with another targeting ligand e.g. a saccharide.

In any of the above aspects, instead of post solid phase synthesis conjugation it is possible to make a preformed Serinol(GN)-phosphoramidite and use this for on-column conjugation.

### Example 1—Synthesis of Conjugates

Example compounds were synthesised according to methods described below and methods known to the person skilled in the art. Assembly of the oligonucleotide chain and linker building blocks was performed by solid phase synthesis applying phosphoramidite methodology. GalNAc conjugation was achieved by peptide bond formation of a GalNAc-carboxylic acid building block to the prior assembled and purified oligonucleotide having the necessary number of amino modified linker building blocks attached.

Oligonucleotide synthesis, deprotection and purification followed standard procedures that are known in the art.

All Oligonucleotides were synthesized on an AKTA oligopilot synthesizer using standard phosphoramidite chemistry. Commercially available solid support and 2′-OMe RNA phosphoramidites, 2′-F, 2′Deoxy RNA phosphoramidites (all standard protection, ChemGenes, LinkTech) and commercially available 3′-Amino Modifier TFA Amino C-6 Icaa CPG 500A (GlyC3Am-CPG, ChemGenes), Fmoc-Amino-DMT C-7 CE phosphoramidite (GlyC3Am-Phos, ChemGenes), 3′-Amino Modifier C-3 Icaa CPG 500A (C3Am-CPG, ChemGenes), Fmoc-Amino-DMT C-3 CED phosphoramidite (C3Am-Phos, ChemGenes), TFA-Amino C-6 CED phosphoramidite (C6Am-Phos, ChemGenes), 3′-Amino-Modifier C7 CPG (C7Am-CPG, Glen Research), non-nucleosidic TFA amino phosphoramidite (PipAm-Phos, AM Chemicals) and non-nucleosidic TFA amino solid support (PipAm-CPG, AM Chemicals) were used. Long trebler phosphoramidite (Itrb-phos) was purchased from Lumiprobe. Per-acetylated galactose amine 8 is commercially available.

Ancillary reagents were purchased from EMP Biotech. Synthesis was performed using a 0.1 M solution of the phosphoramidite in dry acetonitrile and benzylthiotetrazole (BTT) was used as activator (0.3M in acetonitrile). Coupling time was 15 min. A Cap/OX/Cap or Cap/Thio/Cap cycle was applied (Cap: Ac₂O/NMI/Lutidine/Acetonitrile, Oxidizer: 0.1M I₂ in pyridine/H₂O). Phosphorothioates were introduced using standard commercially available thiolation reagent (EDITH, Link technologies). DMT cleavage was achieved by treatment with 3% dichloroacetic acid in toluene. Upon completion of the programmed synthesis cycles a diethylamine (DEA) wash was performed. All oligonucleotides were synthesized in DMT-off mode.

Attachment of the serinol-derived linker moiety was achieved by use of either base-loaded (S)-DMT-Serinol(TFA)-succinate-Icaa-CPG 10 or a (S)-DMT-Serinol(TFA) phosphoramidite 7 (synthesis was performed as described in Hoevelmann et al. (2016)). Tri-antennary GalNAc clusters (ST23/ltrb, ST23/C4XLT or ST23/C6XLT) were introduced by successive coupling of the respective trebler amidite derivatives (Itrb-phos, C4XLT-phos or C6XLT-phos) followed by the GalNAc amidite (ST23-phos).

Attachment of amino modified moieties (non-serinol-derived linkers) was achieved by use of either the respective commercially available amino modified building block CPG or amidite.

The single strands were cleaved off the CPG by 40% aq. methylamine treatment. The resulting crude oligonucleotide was purified by ion exchange chromatography (Resource Q, 6 mL, GE Healthcare) on a AKTA Pure HPLC System using a sodium chloride gradient. Product containing fractions were pooled, desalted on a size exclusion column (Zetadex, EMP Biotech) and lyophilised.

Individual single strands were dissolved in a concentration of 60 OD/mL in H₂O. Both individual oligonucleotide solutions were added together in a reaction vessel. For easier reaction monitoring a titration was performed. The first strand was added in 25% excess over the second strand as determined by UV-absorption at 260 nm. The reaction mixture was heated to 80° C. for 5 min and then slowly cooled to RT. Double strand formation was monitored by ion pairing reverse phase HPLC. From the UV-area of the residual single strand the needed amount of the second strand was calculated and added to the reaction mixture. The reaction was heated to 80° C. again and slowly cooled to RT. This procedure was repeated until less than 10% of residual single strand was detected.

Synthesis of Compounds 2-10

Compounds 2 to 5 and (S)-DMT-Serinol(TFA)-phosphoramidite 7 were synthesised according to literature published methods (Hoevelmann et al. Chem. Sci., 2016,7, 128-135).

### (S)-4-(3-(bis(4-methoxyphenyl)(phenyl)methoxy)-2-(2,2,2-trifluoroacetamido)propoxy)-4-oxobutanoic acid (6)

To a solution of 5 in pyridine was added succinic anhydride, followed by DMAP. The resulting mixture was stirred at room temperature overnight. All starting material was consumed, as judged by TLC. The reaction was concentrated. The crude material was chromatographed in silica gel using a gradient 0% to 5% methanol in DCM (+1% triethylamine) to afford 1.33 g of 6 (yield=38%). m/z (ESI−): 588.2 (100%), (calcd. for C30H29F3NO8⁻ [M−H]⁻ 588.6). 1H-NMR: (400 MHz, CDCl₃) δ [ppm]=7.94 (d, 1H, NH), 7.39-7.36 (m, 2H, CHaryl), 7.29-7.25 (m, 7H, CHaryl), 6.82-6.79 (m, 4H, CHaryl), 4.51-4.47 (m, 1H), 4.31-4.24 (m, 2H), 3.77 (s, 6H, 2×DMTr-OMe), 3.66-3.60 (m, 16H, HNEt₃), 3.26-3.25 (m, 2H), 2.97-2.81 (m, 20H, NEt₃), 2.50-2.41 (4H, m), 1.48-1.45 (m, 26H, HNEt₃), 1.24-1.18 (m, 29H, NEt₃).

### (S)-DMT-Serinol(TFA)-succinate-Icaa-CPG (10)

The (S)-DMT-Serinol(TFA)-succinate (159 mg, 270 umol) and HBTU (113 mg, 299 umol) were dissolved in CH₃CN (10 mL). Diisopropylethylamine (DIPEA, 94 μL, 540 umol) was added to the solution, and the mixture was swirled for 2 min followed by addition native amino-Icaa-CPG (500 A, 3 g, amine content: 136 umol/g). The suspension was gently shaken at room temperature on a wrist-action shaker for 16h then filtered, and washed with DCM and EtOH. The solid support was dried under vacuum for 2 h. The unreacted amines on the support were capped by stirring with acetic anhydride/lutidine/N-methylimidazole at room temperature. The washing of the support was repeated as above. The solid was dried under vacuum to yield solid support 10 (3 g, 26 umol/g loading).

GalNAc Synthon (9)

Synthesis of the GalNAc synthon 9 was performed as described in Nair et al. J. Am. Chem. Soc., 2014, 136 (49), pp 16958-16961, in 46% yield over two steps.

The characterising data matched the published data.

Synthesis of Oligonucleotides

All single stranded oligonucleotides were synthesised according to the reaction conditions described above and in FIGS. 21 and 22, and are outlined in Tables 3 and 4.

All final single stranded products were analysed by AEX-HPLC to prove their purity. Purity is given in % FLP (% full length product) which is the percentage of the UV-area under the assigned product signal in the UV-trace of the AEX-HPLC analysis of the final product. Identity of the respective single stranded products (non-modified, amino-modified precursors or GalNAc conjugated oligonucleotides) was proved by LC-MS analysis.

TABLE 3
Single stranded un-conjugated oligonucleotides

| Product | | | MW (ESI−) | % FLP |
|---|---|---|---|---|
| (11) | Name | MW calc. | found | (AEX-HPLC) |
| A0002 | STS16001A | 6943.3 Da | 6943.0 Da | 86.6% |
| A0006 | STS16001BL4 | 8387.5 Da | 8387.5 Da | 94.1% |
| A0114 | STS22006A | 6143.8 Da | 6143.7 Da | 94.3% |
| A0115 | STS22006BL1 | 7855.1 Da | 7855.1 Da | 92.8% |
| A0122 | STS22009A | 6260.9 Da | 6260.6 Da | 92.8% |
| A0123 | STS22009BL1 | 7783.0 Da | 7782.9 Da | 87.1% |
| A0130 | STS18001A | 6259.9 Da | 6259.8 Da | 76.5% |
| A0131 | STS18001BL4 | 7813.2 Da | 7813.1 Da | 74.3% |
| A0220 | STS16001B-5′1xNH2 | 6982.2 Da | 6982.1 Da | 95.7% |
| A0237 | STS16001A | 6943.3 Da | 6943.3 Da | 95.6% |
| A0244 | STS16001BV1 | 6845.2 Da | 6844.9 Da | 98.2% |
| A0264 | STS16001AV4-3′1xNH2 | 7112.4 Da | 7112.2 Da | 95.4% |
| A0329 | STS16001BV6-3′5′1xNH2 | 7183.3 Da | 7183.2 Da | 88.8% |
| A0560 | STS16001A | 6943.3 Da | 6943.3 Da | 96.7% |
| A0541 | STS16001BV1-3′5′NH2 | 7151.3 Da | 7151.0 Da | 85.6% |
| A0547 | STS16001BV16-3′5′NH2 | 7119.3 Da | 7119.1 Da | 89.9% |
| A0617 | STS16001BV20-3′5′NH2 | 7087.3 Da | 7086.7 Da | 90.1% |
| A0619 | STS16001BV1-3′5′2xNH2 | 7521.3 Da | 7521.3 Da | 93.4% |
| A0680 | STS16001A | 6943.3 Da | 6942.9 Da | 91.2% |
| A0514 | STS22006A | 6143.8 Da | 6143.7 Da | 94.6% |
| A0516 | STS22009BV11-3′5′NH2 | 6665.0 Da | 6664.8 Da | 87.0% |
| A0517 | STS22009BV11-3′5′NH2 | 6593.0 Da | 6593.0 Da | 86.0% |
| A0521 | STS12009BV1-3′5′NH2 | 6437.7 Da | 6437.8 Da | 91.1% |
| A0303 | STS12209BL4 | 7665.0 Da | 7664.9 Da | 90.4% |
| A0304 | STS12209A | 6393.1 Da | 6392.9 Da | 77.6% |
| A0319 | STS22009A | 6260.9 Da | 6260.5 Da | 86.9% |
| A0353 | STS12009A | 6416.1 Da | 6416.1 Da | 94.1% |
| A0216 | STS17001A | 6178.8 Da | 6178.7 Da | 87.2% |
| A0217 | STS17001BL6 | 7937.2 Da | 7937.2 Da | 78.3% |
| A0601 | STS20041A | 6164.9 Da | 6164.5 Da | 95.8% IEX |
| A0602 | STS20041BV1-3′5′NH2 | 6674.0 Da | 6673.6 Da | 90.1% IEX |
| A0605 | STS12009BV58-3′5′NH2 | 6522.0 Da | 6521.8 Da | 91.9% IEX |
| A0782 | STS22006BV16-3′5′NH2 | 6749.3 Da | 6749.2 Da | 83.1% IEX |

5′1×NH₂ means refers to the position (5′ end) and number (1×NH₂) of free serinol derived amino groups which are available for conjugation. For example, 1×3′NH₂ on A0264 means there is free amino group which can be reacted with GalNAc synthon 9 at the 3′ end of the strand A0264. 3′5′1×NH2 means there is one serinol-derived free amino group which can be reacted with GalNAc linker 9 at the 3′ end and the 5′ end of the strand. Similarly, 3′5′1×NH₂ refers to the position (3′ and 5′ end) and number (1×NH₂ each) of free amino groups which are available for conjugation. For example, 3′5′1×NH2 on A0561 means there are 2 free amino group (1 at the 3′ AND 1 at the 5′ end) which can be reacted with GalNAc synthon 9 at the 3′ end of the strand A0561.

TABLE 4
Single stranded oligonucleotides with 5′ and 3′ modifications

| | | | | | | % FLP |
|---|---|---|---|---|---|---|
| | | | | MW | MW (ESI-) | (AEX- |
| Product Name | | 5′mod | 3′mod | calc. | found | HPLC) |
| A0561 | STS16001BV1-3′5′1 × NH2 | C6Am | GlyC3Am | 7267.5 Da | 7267.5 Da | 66.7% |
| A0563 | STS16001BV1-3′5′1 × NH2 | C3Am | C3Am | 7183.4 Da | 7183.1 Da | 75.1% |
| A0651 | STS16001BV1-3′5′1 × NH2 | C6Am | C7Am | 7265.6 Da | 7265.2 Da | 99.6% |
| A0653 | STS16001BV1-3′5′1 × NH2 | GlyC3Am | GlyC3Am | 7299.5 Da | 7299.3 Da | 88.1% |
| A0655 | STS16001BV1-3′5′1 × NH2 | PipAm | PipAm | 7517.7 Da | 7517.5 Da | 89.8% |
| A0735 | STS16001BV1-3′5′1 × NH2 | Asp | Asp | 7563.3 Da | 7561.5 Da | 94.9% |
| A0843 | STS16001BV1-3′5′1 × NH2 | HPro | HPro | 7461.7 Da | 7461.6 Da | 96.4% |
| A0844 | STS16001BV1-3′5′1 × NH2 | SerC6 | SerC6 | 7409.7 Da | 7409.6 Da | 93.6% |

Synthesis of Certain Conjugates of the Invention and Reference Conjugates 1-2

Conjugation of the GalNac synthon (9) was achieved by coupling to the serinol-amino function of the respective oligonucleotide strand 11 using a peptide coupling reagent. Therefore, the respective amino-modified precursor molecule 11 was dissolved in H₂O (500 OD/mL) and DMSO (DMSO/H₂O, 2/1, v/v) was added, followed by DIPEA (2.5% of total volume). In a separate reaction vessel pre-activation of the GalN(Ac4)-13 C4-acid (9) was performed by reacting 2 eq. (per amino function in the amino-modified precursor oligonucleotide 11) of the carboxylic acid component with 2 eq. of HBTU in presence of 8 eq. DIPEA in DMSO. After 2 min the pre-activated compound 9 was added to the solution of the respective amino-modified precursor molecule. After 30 min the reaction progress was monitored by LCMS or AEX-HPLC. Upon completion of the conjugation reaction the crude product was precipitated by addition of 10×iPrOH and 0.1×2M NaCl and harvested by centrifugation and decantation. To set free the acetylated hydroxyl groups in the GalNAc moieties the resulting pellet was dissolved in 40% MeNH2 (1 mL per 500 OD) and after 15 min at RT diluted in H₂O (1:10) and finally purified again by anion exchange and size exclusion chromatography and lyophilised to yield the final product 12 (Table 5).

TABLE 5
Single stranded GalNAc-conjugated oligonucleotides

| Product | Starting | | | MW (ESI−) | % FLP |
|---|---|---|---|---|---|
| (12) | Material | Name | MW calc. | found | (AEX-HPLC) |
| A0241 | A0220 | STS16001BL20 | 7285.5 Da | 7285.3 Da | 91.8% |
| A0268 | A0264 | STS16001AV4L33 | 7415.7 Da | 7415.4 Da | 96.9% |
| A0330 | A0329 | STS16001BV6L42 | 7789.8 Da | 7789.8 Da | 95.5% |
| A0544 | A0541 | STS16001BV1L75 | 7757.9 Da | 7757.7 Da | 93.3% |
| A0550 | A0547 | STS16001BV16L42 | 7725.9 Da | 7725.7 Da | 88.5% |
| A0620 | A0617 | STS16001BV20L75 | 7693.91 Da | 7693.2 Da | 90.9% |
| A0622 | A0619 | STS16001BV1L94 | 8734.3 Da | 8734.6 Da | 82.9% |
| A0519 | A0516 | STS22006BV11L42 | 7271.7 Da | 7271.7 Da | 90.0% |
| A0520 | A0517 | STS22009BV11L42 | 7199.6 Da | 7199.7 Da | 92.9% |
| A0522 | A0521 | STS12009BV1L42 | 7044.4 Da | 7044.4 Da | 96.0% |
| A0603 | A0602 | STS20041BV1L42 | 7280.7 Da | 7280.4 Da | 93.4% |
| A0608 | A0605 | STS12009BV58L42 | 7128.8 Da | 7128.3 Da | 95.0% |
| A0783 | A0782 | STS22006BV16L42 | 7356.0 Da | 7355.7 Da | 91.4% |

Synthesis of Certain Conjugates of the Invention

Conjugation of the GalNac synthon (9) was achieved by coupling to the amino function of the respective oligonucleotide strand 14 using a peptide coupling reagent. Therefore, the respective amino-modified precursor molecule 14 was dissolved in H₂O (500 OD/mL) and DMSO (DMSO/H₂O, 2/1, v/v) was added, followed by DIPEA (2.5% of total volume). In a separate reaction vessel pre-activation of the GalN(Ac4)—C4-acid (9) was performed by reacting 2 eq. (per amino function in the amino-modified precursor oligonucleotide 14) of the carboxylic acid component with 2 eq. of HBTU in presence of 8 eq. DIPEA in DMSO. After 2 min the pre-activated compound 9 was added to the solution of the respective amino-modified precursor molecule. After 30 min the reaction progress was monitored by LCMS or AEX-HPLC. Upon completion of the conjugation reaction the crude product was precipitated by addition of 10×iPrOH and 0.1×2M NaCl and harvested by centrifugation and decantation. To set free the acetylated hydroxyl groups in the GalNAc moieties the resulting pellet was dissolved in 40% MeNH2 (1 mL per 500 OD) and after 15 min at RT diluted in H₂O (1:10) and finally purified again by anion exchange and size exclusion chromatography and lyophilised to yield the final product 15 (Table 6).

TABLE 6
Single stranded GalNAc-conjugated oligonucleotides

| Product | Starting | | | MW (ESI−) | % FLP |
|---|---|---|---|---|---|
| (15) | Material | Name | MW calc. | found | (AEX-HPLC) |
| A0562 | A0561 | STS16001BV1L87 | 7874.2 Da | 7874.0 Da | 82.7% |
| A0564 | A0563 | STS16001BV1L88 | 7790.0 Da | 7789.4 Da | 90.4% |
| A0652 | A0651 | STS16001BV1L96 | 7872.2 Da | 7871.8 Da | 94.6% |
| A0654 | A0653 | STS16001BV1L97 | 7906.2 Da | 7905.6 Da | 89.9% |
| A0656 | A0655 | STS16001BV1L98 | 8124.3 Da | 8124.0 Da | 93.6% |
| A0775 | A0735 | STS16001BV1L110 | 7818.0 Da | 7818.1 Da | 96.3% |
| A0845 | A0843 | STS16001BV1L114 | 8068.4 Da | 8068.4 Da | 97.1% |
| A0846 | A0844 | STS16001BV1L115 | 8016.2 Da | 8016.3 Da | 96.0% |

Double Strand Formation

Double strand formation was performed according to the methods described above. The double strand purity is given in % double strand which is the percentage of the UV-area under the assigned product signal in the UV-trace of the IP-RP-HPLC analysis (Table 7).

TABLE 7
Nucleic acid conjugates

| | Starting Materials | | | |
|---|---|---|---|---|
| Product | First Strand | Second Strand | Name | % double strand |
| Ref. Conj. 1 | A0237 | A0241 | STS16001L20 | 97.7% |
| Ref. Conj. 2 | A0268 | A0244 | STS16001L33 | 97.8% |
| Ref. Conj. 3 | A0130 | A0131 | STS18001L4 | 96.8% |
| Ref. Conj. 4 | A0002 | A0006 | STS16001L4 | 90.1% |
| Ref. Conj. 5 | A0216 | A0217 | STS17001L6 | 88.4% |
| Ref. Conj. 6 | A0114 | A0115 | STS22006L1 | 85.6% |
| Ref. Conj. 7 | A0122 | A0123 | STS22009L1 | 96.4% |
| Ref. Conj. 8 | A0304 | A0303 | STS12209L4 | 93.0% |
| Ref. Conj. 9 | | | STS12009L4 | |
| Conjugate 1 | A0268 | A0241 | STS16001L24 | 96.0% |
| Conjugate 2 | A0237 | A0330 | STS16001V1L42 | 98.5% |
| Conjugate 3 | A0268 | A0330 | STS16001V1L43 | 98.2% |
| Conjugate 4 | A0560 | A0544 | STS16001V1L75 | 92.5% |
| Conjugate 5 | A0560 | A0550 | STS16001V16L42 | 95.3% |
| Conjugate 6 | A0237 | A0620 | STS16001V20L75 | 97.8% |
| Conjugate 7 | A0237 | A0622 | STS16001V1L94 | 93.7% |
| Conjugate 8 | A0680 | A0652 | STS16001V1L96 | 98.4% |
| Conjugate 9 | A0680 | A0654 | STS16001V1L97 | 95.8% |
| Conjugate 10 | A0680 | A0656 | STS16001V1L98 | 97.6% |
| Conjugate 11 | A0560 | A0564 | STS16001V1L88 | 95.0% |
| Conjugate 12 | A0237 | A0562 | STS16001V1L87 | 96.8% |
| Conjugate 15 | A0514 | A0519 | STS22006V11L42 | 98.6% |
| Conjugate 16 | A0319 | A0520 | STS22009V11L42 | 97.0% |
| Conjugate 18 | A0353 | A0522 | STS12009V1L42 | 98.0% |
| Conjugate 19 | A0601 | A0603 | STS20041BL42 | 97.6% |
| Conjugate 20 | A0680 | A0775 | STS16001L110 | 97.9% |
| Conjugate 21 | A0680 | A0845 | STS16001L115 | 97.6% |
| Conjugate 22 | A0680 | A0846 | STS16001L116 | 97.9% |
| Conjugate 23 | A0610 | A0783 | STS22006V16L42 | 96.0% |
| Conjugate 24 | A0443 | A0608 | STS12009V58L42 | 97.7% |

Sequences

Modifications key for the following sequences:

f denotes 2′-F 2′deoxyribonucleotide or 2′-F ribonucleotide (the terms are interchangeable)

m denotes 2′-OMe ribonucleotide

(ps) denotes phosphorothioate linkage

### Definitions

Ser(GN) is a GalNAc-C₄ building block attached to serinol derived linker moiety:

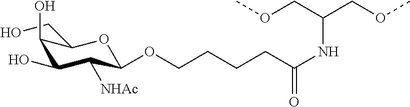

wherein the O— is the linkage between the oxygen atom and e.g. H, phosphodiester linkage or phosphorothioate linkage.

GN is:

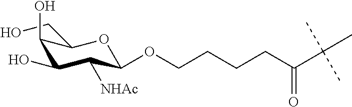

C4XLT (also known as ST41) is:

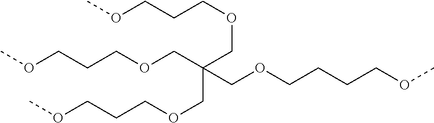

C6XLT (also known as ST43) is:

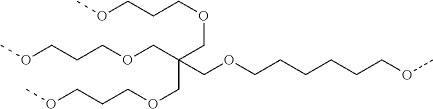

ST23 is:

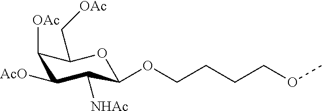

Synthesis of the phosphoramidite derivatives of C4XLT (C4XLT-phos) and C6XLT (C6XLT-phos) as well as ST23 (ST23-phos) can be performed as described in WO2017/174657.

C4XLT-phos:

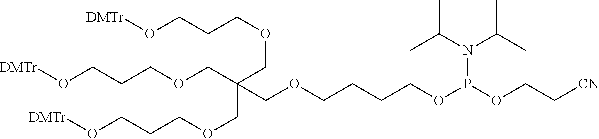

ST23-phos:

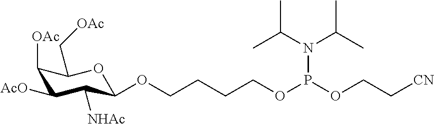

C6XLT-phos:

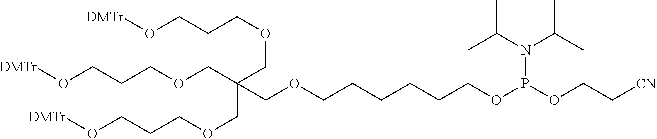

Itrb-phos:

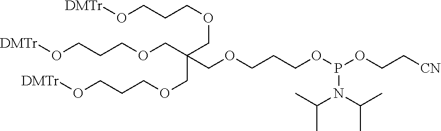

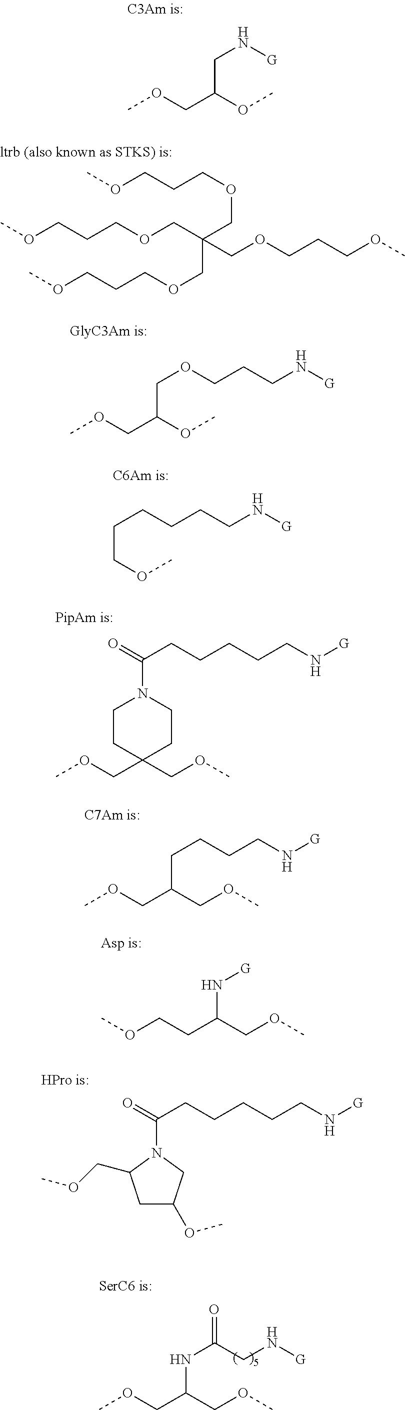

wherein G=H (pre conjugation) or G=GN (post conjugation).

Conjugate 1

Antisense strand—STS16001AL33

5′ mU (ps) fU (ps) mA fU mA fG mA fG mC fA mA fG mA fA mC fA mC fU mG (ps) fU (ps) mU (ps) Ser(GN) 3′

Sense strand—STS16001BL20

5′ Ser(GN) (ps) fA mA fC mA fG mU fG mU fU mC fU mU fG mC fU mC fU mA fU (ps) mA (ps) fA 3′

Conjugate 2

Antisense strand—STS16001A

mU (ps) fU (ps) mA fU mA fG mA fG mC fA mA fG mA fA mC fA mC fU mG (ps) fU (ps) mU

Sense strand—STS16001BV1L42

Ser(GN) (ps) fA (ps) mA (ps) fC mA fG mU fG mU fU mC fU mU fG mC fU mC fU mA fU (ps) mA (ps) fA (ps) Ser(GN)

Conjugate 3

Antisense strand—STS16001AL33

5′ mU (ps) fU (ps) mA fU mA fG mA fG mC fA mA fG mA fA mC fA mC fU mG (ps) fU (ps) mU (ps) Ser(GN) 3′

Sense strand—STS16001BV1L42

5′ Ser(GN) (ps) fA (ps) mA (ps) fC mA fG mU fG mU fU mC fU mU fG mC fU mC fU mA fU (ps) mA (ps) fA (ps) Ser(GN) 3′

Conjugate 4

Antisense strand—STS16001A

mU (ps) fU (ps) mA fU mA fG mA fG mC fA mA fG mA fA mC fA mC fU mG (ps) fU (ps) mU

Sense strand—STS16001BV1L75

5′ Ser(GN) fA (ps) mA (ps) fC mA fG mU fG mU fU mC fU mU fG mC fU mC fU mA fU (ps) mA (ps) fA Ser(GN) 3′

Conjugate 5

Antisense strand—STS16001A

mU (ps) fU (ps) mA fU mA fG mA fG mC fA mA fG mA fA mC fA mC fU mG (ps) fU (ps) mU

Sense strand—STS16001BV16L42

5′ Ser(GN) (ps) fA mA fC mA fG mU fG mU fU mC fU mU fG mC fU mC fU mA fU mA fA (ps) Ser(GN) 3′

Conjugate 6

Antisense strand—STS16001A

mU (ps) fU (ps) mA fU mA fG mA fG mC fA mA fG mA fA mC fA mC fU mG (ps) fU (ps) mU

Sense strand—STS16001BV20L75

5′ Ser(GN) fA mA fC mA fG mU fG mU fU mC fU mU fG mC fU mC fU mA fU mA fA Ser(GN) 3′

Conjugate 7

Antisense strand—STS16001A

mU (ps) fU (ps) mA fU mA fG mA fG mC fA mA fG mA fA mC fA mC fU mG (ps) fU (ps) mU

Sense strand—STS16001BV1L94

5′ Ser(GN) (ps) Ser(GN) (ps) fA (ps) mA (ps) fC mA fG mU fG mU fU mC fU mU fG mC fU mC fU mA fU (ps) mA (ps) fA (ps) Ser(GN) (ps) Ser(GN) 3′

Conjugate 8

Antisense strand—STS16001A 5′ mU (ps) fU (ps) mA fU mA fG mA fG mC fA mA fG mA fA mC fA mC fU mG (ps) fU (ps) mU 3′

Sense strand—STS16001V1BL96

5′ C₆Am(GN) (ps) fA (ps) mA (ps) fC mA fG mU fG mU fU mC fU mU fG mC fU mC fU mA fU (ps) mA (ps) fA (ps) C₇Am(GN) 3′

Conjugate 9

Antisense strand—STS16001A

5′ mU (ps) fU (ps) mA fU mA fG mA fG mC fA mA fG mA fA mC fA mC fU mG (ps) fU (ps) mU 3′

Sense strand—STS16001V1BL97

5′ GlyC3Am(GN) (ps) fA (ps) mA (ps) fC mA fG mU fG mU fU mC fU mU fG mC fU mC fU mA fU (ps) mA (ps) fA (ps) GlyC3Am(GN) 3′

Conjugate 10

Antisense strand—STS16001A

5′ mU (ps) fU (ps) mA fU mA fG mA fG mC fA mA fG mA fA mC fA mC fU mG (ps) fU (ps) mU 3′

Sense strand—STS16001V1BL98

5′ PipAm(GN) (ps) fA (ps) mA (ps) fC mA fG mU fG mU fU mC fU mU fG mC fU mC fU mA fU (ps) mA (ps) fA (ps) PipAm(GN) 3′

Conjugate 11

Antisense strand—STS16001A

5′ mU (ps) fU (ps) mA fU mA fG mA fG mC fA mA fG mA fA mC fA mC fU mG (ps) fU (ps) mU 3′

Sense strand—STS16001V1BL88

5′ C3Am(GN) (ps) fA (ps) mA (ps) fC mA fG mU fG mU fU mC fU mU fG mC fU mC fU mA fU (ps) mA (ps) fA (ps) C₃Am(GN) 3′

Conjugate 12

Antisense strand—STS16001A

5′ mU (ps) fU (ps) mA fU mA fG mA fG mC fA mA fG mA fA mC fA mC fU mG (ps) fU (ps) mU 3′

Sense strand—STS16001V1BL87

5′ C6Am(GN) (ps) fA (ps) mA (ps) fC mA fG mU fG mU fU mC fU mU fG mC fU mC fU mA fU (ps) mA (ps) fA (ps) GlyC3Am(GN) 3′

Conjugate 15

Antisense strand—STS22006A

mU (ps) fC (ps) mU fU mC fU mU fA mA fA mC fU mG fA mG fU mU (ps) fU (ps) mC

Sense strand—STS22006BV11L42

Ser(GN) (ps) fG (ps) mA (ps) fA mA fC mU fC mA fG mU fU mU fA mA fG mA fA (ps) mG (ps) fA (ps) Ser(GN)

Conjugate 16

Antisense strand—STS22009A

mA (ps) fU (ps) mG fU mA fG mC fC mG fA mG fG mA fU mC fU mU (ps) fC (ps) mU

Sense strand—STS22009BV11L42

Ser(GN) (ps) fA (ps) mG (ps) fA mA fG mA fU mC fC mU fC mG fG mC fU mA fC (ps) mA (ps) fU (ps) Ser(GN)

Conjugate 18

Antisense strand—STS12009A

mA (ps) fA (ps) mC fC mA fG mA fA mG fA mA fG mC fA mG fG mU (ps) fG (ps) mA

Sense strand—STS12009BV1L42

Ser(GN) (ps) fU (ps) mC (ps) fA mC fC mU fG mC fU mU fC mU fU mC fU mG fG (ps) mU (ps) fU (ps) Ser(GN)

Conjugate 19

Antisense strand—STS20041A

mA (ps) fU (ps) mA fA mC fU mC fU mG fU mC fC mA fU mU fA mC (ps) fC (ps) mG

Sense strand—STS20041BV1L42

Ser(GN) (ps) fC (ps) mG (ps) fG mU fA mA fU mG fG mA fC mA fG mA fG mU fU (ps) mA (ps) fU (ps) Ser(GN)

Conjugate 20

Antisense strand—STS16001A

mU (ps) fU (ps) mA fU mA fG mA fG mC fA mA fG mA fA mC fA mC fU mG (ps) fU (ps) mU

Sense strand—STS16001BV1L110

Asp(GN) (ps) fA (ps) mA (ps) fC mA fG mU fG mU fU mC fU mU fG mC fU mC fU mA fU (ps) mA (ps) fA (ps) Asp(GN)

Conjugate 21

Antisense strand—STS16001A

mU (ps) fU (ps) mA fU mA fG mA fG mC fA mA fG mA fA mC fA mC fU mG (ps) fU (ps) mU

Sense strand—STS16001BV1L115

Hpro(GN) (ps) fA (ps) mA (ps) fC mA fG mU fG mU fU mC fU mU fG mC fU mC fU mA fU (ps) mA (ps) fA (ps) Hpro(GN)

Conjugate 22

Antisense strand—STS16001A

mU (ps) fU (ps) mA fU mA fG mA fG mC fA mA fG mA fA mC fA mC fU mG (ps) fU (ps) mU

Sense strand—STS16001BV1L116

SerC6(GN) (ps) fA (ps) mA (ps) fC mA fG mU fG mU fU mC fU mU fG mC fU mC fU mA fU (ps) mA (ps) fA (ps) SerC6(GN)

Conjugate 23

Antisense strand—STS22006A

mU (ps) fC (ps) mU fU mC fU mU fA mA fA mC fU mG fA mG fU mU (ps) fU (ps) mC sense strand—STS22006BV16L42

Ser(GN) (ps) mG (ps) mA (ps) mA mA mC mU fC fA fG mU mU mU mA mA mG mA mA (ps) mG (ps) mA (ps) Ser(GN)

Conjugate 24

Antisense strand—STS12009A

mA (ps) fA (ps) mC fC mA fG mA fA mG fA mA fG mC fA mG fG mU (ps) fG (ps) mA

Sense strand—STS12009BV58L42

Ser(GN) (ps) mU (ps) mC (ps) mA mC mC mU fG fC fU mU mC mU mU mC mU mG mG (ps) mU (ps) mU (ps) Ser(GN)

Reference Conjugate 1

Antisense strand—STS16001A

mU (ps) fU (ps) mA fU mA fG mA fG mC fA mA fG mA fA mC fA mC fU mG (ps) fU (ps) mU

Sense strand—STS16001BL20

Ser(GN) (ps) fA mA fC mA fG mU fG mU fU mC fU mU fG mC fU mC fU mA fU (ps) mA (ps) fA

Reference Conjugate 2

Antisense strand—STS16001AL33

mU (ps) fU (ps) mA fU mA fG mA fG mC fA mA fG mA fA mC fA mC fU mG (ps) fU (ps) mU (ps) Ser(GN)

Sense strand—STS16001BV1

fA (ps) mA (ps) fC mA fG mU fG mU fU mC fU mU fG mC fU mC fU mA fU (ps) mA (ps) fA

Reference Conjugate 3—“Luc”

Antisense strand—STS18001A

mU (ps) fC (ps) mG fA mA fG mU fA mU fU mC fC mG fC mG fU mA (ps) fC (ps) mG

Sense strand—STS18001BL4

[(ST23) (ps)]3 C₄XLT (ps) fC mG fU mA fC mG fC mG fG mA fA mU fA mC fU mU fC (ps) mG (ps) fA

Reference Conjugate 4

Antisense strand—STS16001A

mU (ps) fU (ps) mA fU mA fG mA fG mC fA mA fG mA fA mC fA mC fU mG (ps) fU (ps) mU

Sense strand—STS16001BL4

5′[(ST23) (ps)]3 C₄XLT(ps) fA (ps) mA (ps) fC mA fG mU fG mU fU mC fU mU fG mC fU mC fU mA fU (ps) mA (ps) fA

Reference Conjugate 5—“Ctr”

Antisense strand—STS17001A

mC (ps) fU (ps) mU fA mC fU mC fU mC fG mC fC mC fA mA fG mC (ps) fG (ps) mA

Sense strand—STS17001BL6

[(ST23) (ps)]3 (C₆XLT) (ps) fU mC fG mC fU mU fG mG fG mC fG mA fG mA fG mU fA (ps) mA (ps) fG

Reference Conjugate 6

Antisense strand—STS22006A

mU (ps) fC (ps) mU fU mC fU mU fA mA fA mC fU mG fA mG fU mU (ps) fU (ps) mC

Sense strand—STS22006BL1

[ST23 (ps)]3 Itrb (ps) fG mA fA mA fC mU fC mA fG mU fU mU fA mA fG mA fA (ps) mG (ps) fA

Reference Conjugate 7

Antisense strand—STS22009A

mA (ps) fU (ps) mG fU mA fG mC fC mG fA mG fG mA fU mC fU mU (ps) fC (ps) mU

Sense strand—STS22009BL1

[ST23 (ps)]3 Itrb (ps) fA mG fA mA fG mA fU mC fC mU fC mG fG mC fU mA fC (ps) mA (ps) fU

Reference Conjugate 8

Antisense strand—STS12209A

mU (ps) fA (ps) mC fC mA fG mA fA mG fA mA fG mC fA mG fG mU (ps) fG (ps) mA

Sense strand—STS12209BL4

[ST23 (ps)]3 ST41 (ps) fU mC fA mC fC mU fG mC fU mU fC mU fU mC fU mG fG (ps) mU (ps) fA

Reference Conjugate 9

Antisense strand—STS12009A

mA (ps) fA (ps) mC fC mA fG mA fA mG fA mA fG mC fA mG fG mU (ps) fG (ps) mA

Sense strand—STS12009BL4

[ST23 (ps)]3 ST41 (ps) fU mC fA mC fC mU fG mC fU mU fC mU fU mC fU mG fG (ps) mU (ps) fU

### Example 2—In Vitro Determination of TTR Knockdown of Various TTR siRNA GalNAc Conjugates

Conjugates 1 to 3

Murine primary hepatocytes were seeded into collagen pre-coated 96 well plates (Thermo Fisher Scientific, #A1142803) at a cell density of 30,000 cells per well and treated with siRNA-conjugates at concentrations ranging from 10 nM to 0.0001 nM. 24h post treatment cells were lysed and RNA extracted with InviTrap® RNA Cell HTS 96 Kit/C24×96 preps (Stratec #7061300400) according to the manufactures protocol. Transcripts levels of TTR and housekeeping mRNA (PtenII) were quantified by TaqMan analysis.

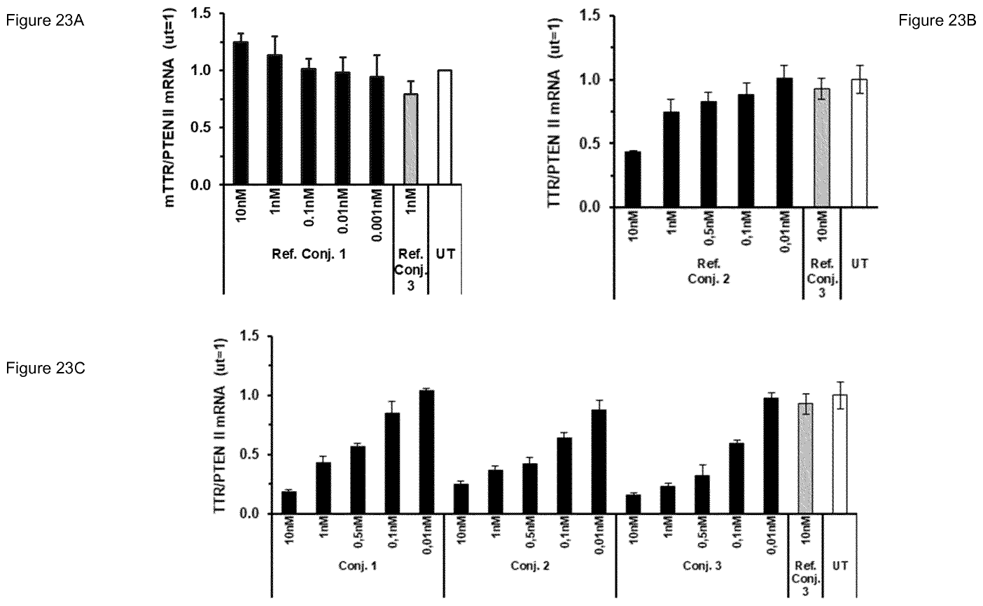

Target gene expression in primary murine hepatocytes 24h following treatment at 0.01 nM, 0.1 nM, 0.5 nM, 1 nM and 10 nM with the conjugates of the invention, Conjugates 1-3, showed that target gene expression decreases as the dose of the conjugate increased compared to the negative controls (see “UT” column and Reference Conjugate 3), as shown in FIG. 23. This indicates that the first strand is binding to the target gene, thus lowering gene expression. FIG. 23 also shows the target gene expression levels of Reference Conjugates 1 and 2 which act as comparator conjugates. As can be seen from a comparison between the data presented in FIGS. 23A and 23C, and 23B and 23C, the conjugates of the invention (Conjugates 1-3) decrease the target gene expression compared to Reference Conjugates 1 and 2. The most effective conjugate at 0.01 nM appears to be Conjugate 2. The most effective conjugate at 0.1 nM, 0.5 nM, 1 nM and 10 nM appears to be Conjugate 3.

Conjugates 4 to 7

The method described above under “In vitro experiments” in the General Method section was followed.

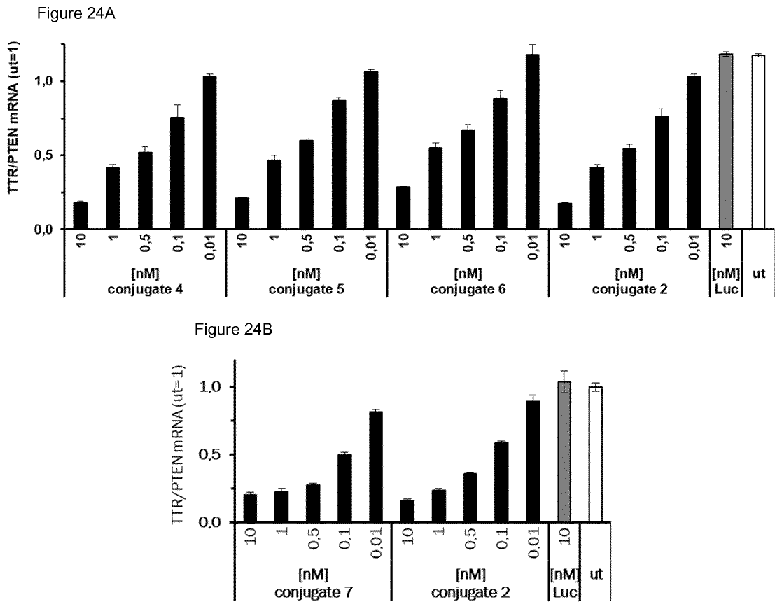

Target gene expression in primary murine hepatocytes 24h following treatment at 0.01 nM, 0.1 nM, 0.5 nM, 1 nM and 10 nM with the conjugates of the invention, Conjugates 4-7, showed that target gene expression decreases as the dose of the conjugate increased compared to the negative controls (see “UT” column and Luc [Reference Conjugate 3]), as shown in FIG. 24. This indicates that the first strand is binding to the target gene, thus lowering gene expression.

The in vitro data show that in the context of one or two serinol-derived linker moieties being provided at 5′ and 3′ ends of the sense strand in Conjugates 4-7, the number of phosphorothioate (PS) bonds between the terminal nucleotide and the linker, and/or between the terminal three nucleotides in the sense strand, can be varied whilst maintaining efficacy for decreasing target gene expression.

Conjugates 8 to 12 and 19

The method described above under “In vitro experiments” in the General Method section was followed.

Target gene expression in primary murine hepatocytes 24h following treatment at 0.01 nM, 0.1 nM, 0.5 nM, 1 nM and 10 nM with the conjugates of the invention, Conjugates 8-12, showed that target gene expression decreases as the dose of the conjugate increased compared to the negative controls (see “UT” column and Luc [Reference Conjugate 3]), as shown in FIG. 25. This indicates that the first strand is binding to the target gene, thus lowering gene expression. In particular, Conjugates 8, 9, 10 and 11 appear to be comparable to or better than Conjugate 2 which was previously shown to be the most effective conjugate at 0.01 nM.

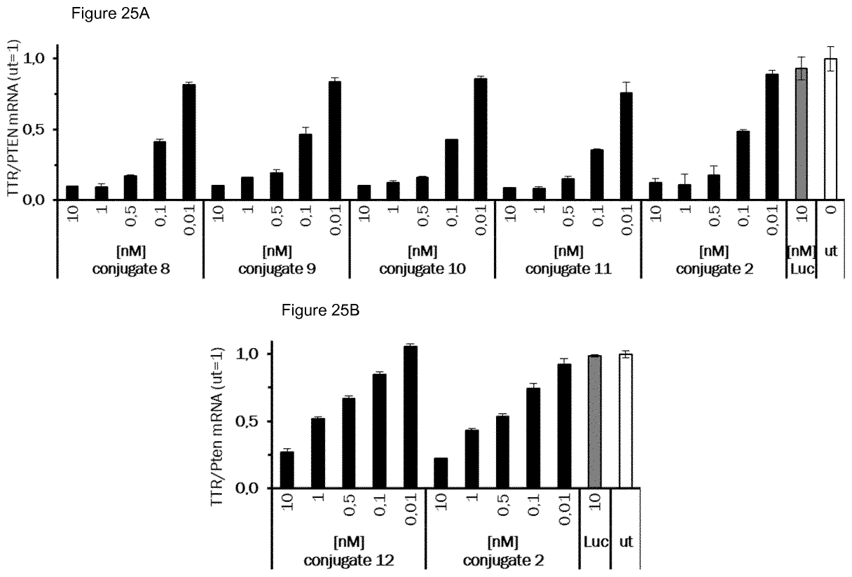

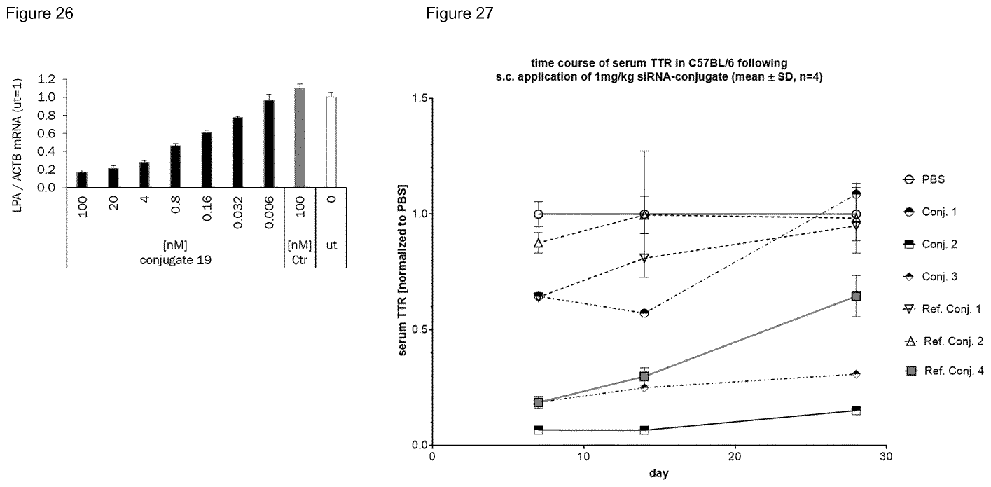

Conjugate 19 was also shown to decrease target gene expression compared to the negative controls (see “UT” column and Ctr which is a non-targeting siRNA and also referred to as Reference Conjugate 5), as shown in FIG. 26. This indicates that the first strand is binding to the target gene, thus lowering gene expression. Conjugate 19 targets the gene LPA, whereas the other Conjugates tested above target TTR. This indicates that the linker moieties allow efficient gene expression lowering not only of a particular gene but of different genes. In other words, the linker moieties can be used for different siRNA sequences.

The in vitro data for Conjugates 8-12 and 19 show that a number of linkers which are structurally diverse and which are conjugated at both termini of the sense strand are effective at decreasing target gene expression. Conjugates 8-12 and 19 decrease target gene expression more effectively than “Luc” which is Reference Conjugate 3 (for Conjugates 8-12), “Ctr” which is Reference Conjugate 5 (for Conjugate 19) and untreated control.

Conjugates 20 to 22

The method described above under “In vitro experiments” in the General Method section was followed.

Target gene expression in primary murine hepatocytes 24h following treatment at 0.01 nM, 0.1 nM, 0.5 nM, 1 nM and 10 nM with the conjugates of the invention, Conjugates 20-22 with alternative linkers, showed similar that target gene expression decreases as the dose of the conjugate increased as compared the positive control conjugate 2 whereas the negative controls (see “UT” column and Luc [Reference Conjugate 3]) don't show target gene reduction, as shown in FIG. 35.

The in vitro data show that in the context of two linker moieties one at 5′ and one at the 3′ end of the sense strand in Conjugates 2, and 20-22, the nature of the linker can be varied whilst maintaining efficacy for decreasing target gene expression.

### Example 3—In Vivo Time Course of Serum TTR, ALDH2 and TMPRSS6 in Mice

Conjugates 1 to 3

C57BL/6 mice were treated s.c. with 1 mg/kg siRNA-conjugates at day 0. Serum samples were taken at day 7, 14, and 27 by orbital sinus bleeding and stored at −20° C. until analysis. Serum TTR quantification was performed with a Mouse Prealbumin ELISA (ALPCO, 41-PALMS/lot 22, 2008003B) according to the manufacturers protocol (sample dilution 1:8000 or 1:800).

The results of the time course of serum TTR in c57BL/6 mice cohorts of n=4 at 7, 14, and 27 days post s.c. treatment with 1 mg/kg Conjugates 1-3, Reference Conjugates 1, 2 and 4, and mock treated (PBS) individuals is shown in FIG. 27. As indicated by the data in FIG. 27, the conjugates of the invention are particularly effective at reducing target gene expression compared to the negative control (PBS) and Reference Conjugates 1, 2, and in particular to Reference Conjugate 4. Conjugates 2 and 3 are also more effective than Reference Conjugates 1, 2 and 4. The most effective conjugate is Conjugate 2. Thus, it may be expected that the dosing level of Conjugate 2 would be about three times lower to achieve the same initial knock down and would also result in longer duration of knock down as compared to Reference Conjugate 4.

More specifically, Conjugate 2 resulted in 3-fold lower target protein level in serum at day seven and 4-fold lower target protein level in serum at day 27 compared to Reference Conjugate 4 at equimolar dose in wild type mice. Furthermore, Conjugate 2 resulted in 85% reduction of target serum protein level at day 27 after single injection, compared to 36% reduction by equimolar amount of Reference Conjugate 4.

Conjugates 15 to 18

The method described above under “In vivo experiments” in the General Method section was followed.

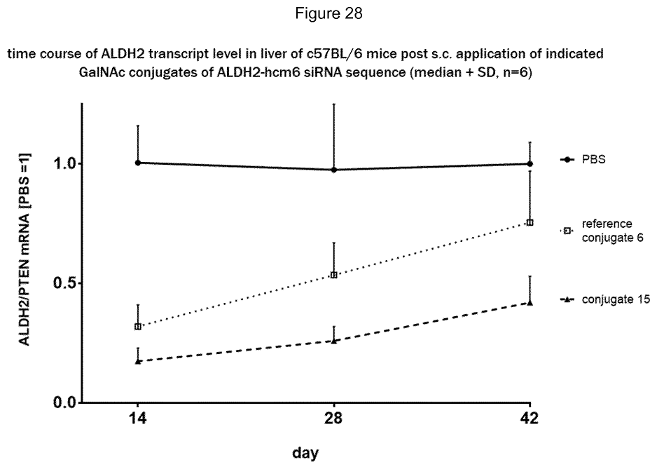

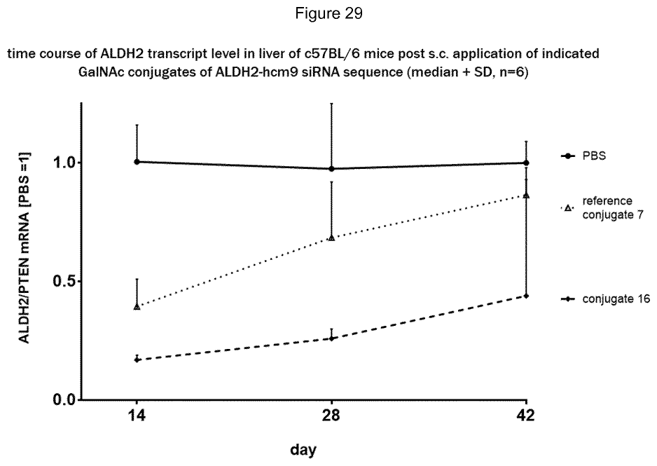

The results of the time course of ALDH2 transcript level in liver in c57BL/6 mice cohorts of n=6 at 14, 28 and 42 days post s.c. treatment with 1 mg/kg Conjugates 15 and 16, Reference Conjugates 6 and 7, and mock treated (PBS) individuals is shown in FIGS. 28 and 29. As indicated by the data in FIGS. 28 and 29, the conjugates of the invention are particularly effective at reducing target gene expression compared to the negative control (PBS) and Reference Conjugates 6 and 7 respectively.

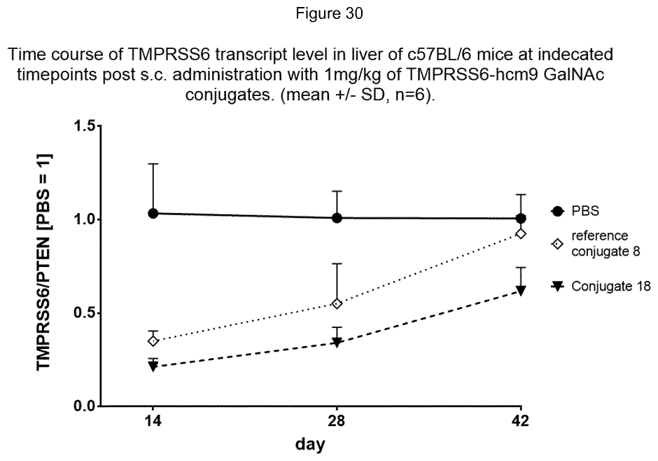

The results of the time course of TMPRSS6 transcript level in liver in c57BL/6 mice cohorts of n=6 at 14, 28, and 42 days post s.c. treatment with 1 mg/kg Conjugate 18, Reference Conjugate 8, and mock treated (PBS) individuals is shown in FIG. 30. As indicated by the data in FIG. 30, the conjugates of the invention are particularly effective at reducing target gene expression compared to the negative control (PBS) and Reference Conjugate 8.

Overall, the in vivo data show that a variety of example linkers which are conjugated at both termini of the second strand are effective at decreasing target gene expression in vivo. The positioning of the linker improves in vivo potency of conjugates, as compared to a triantennary GalNAc-linker control at the 5′ terminus of the second strand (Reference Conjugates 6, 7 and 8). This is surprising, because the target receptor for GalNAc is present at the surface of hepatocytes in clusters of three receptors. It is therefore unexpected that an siRNA with only two GalNAc moieties that are located far from each other on the siRNA are well taken up into cells. The data also show that the linkers allow to effectively down regulate a variety of genes and that the effect is therefore not dependent on a specific siRNA sequence.

Conjugates 7 and 2

The method described above under “In vivo experiments” in the General Method section was followed.

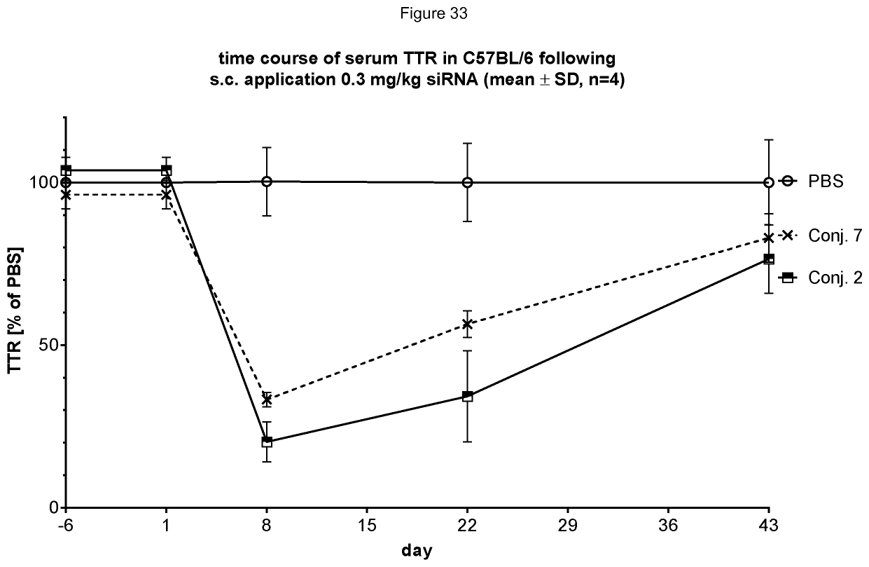

The time course of serum TTR in FIG. 33 show a superior in vivo target knock down for Conjugate 2 as compared to Conjugate 7. Overall, these data demonstrate that the GalNAc-linker configuration and valency of Conjugate 2 with single serinol-GalNAc at the 3′- and 5′-end of either sense strand is preferred over that of Conjugate 7 with two serinol-GalNAc at both 3′- and 5′-end of the sense strand.

### Example 4—Stability Studies

The method described above under “Tritosome stability assay” in the General Method section was followed.

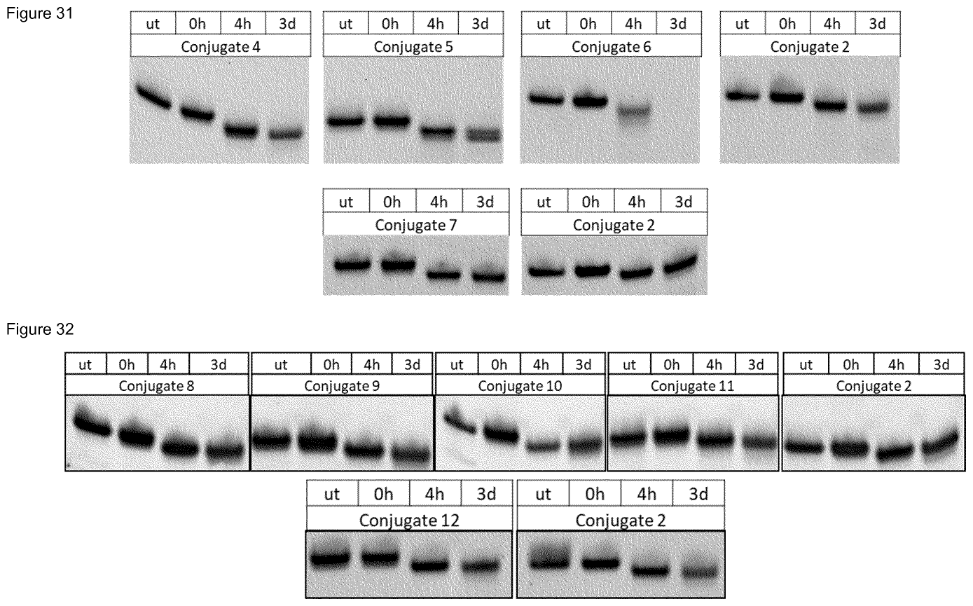

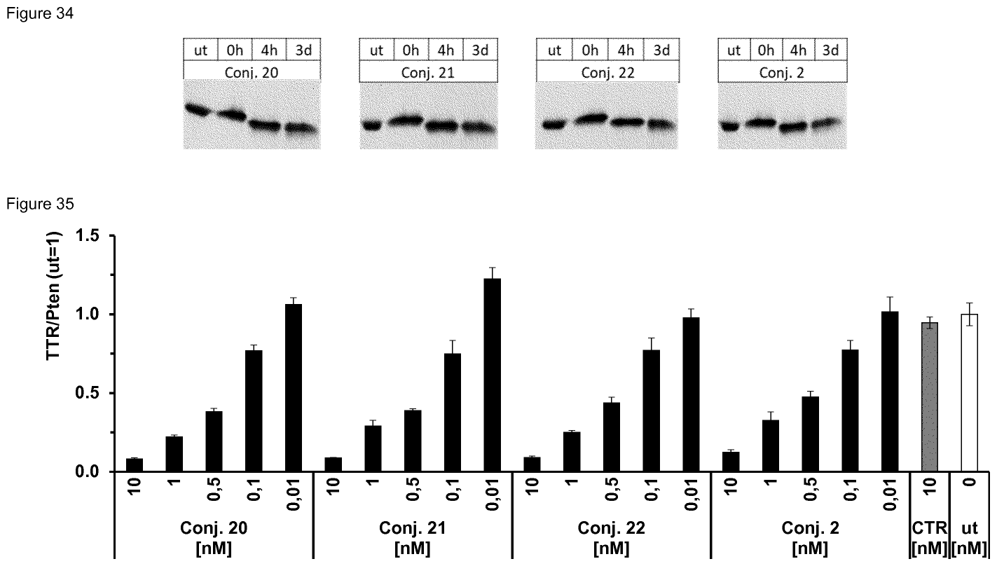

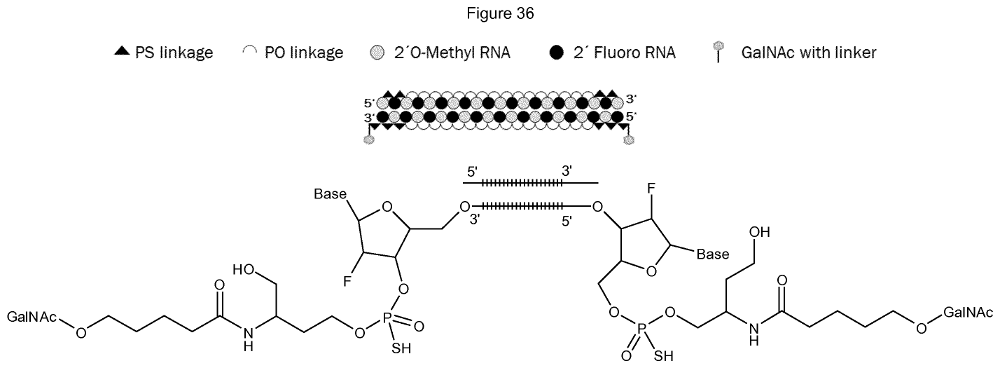

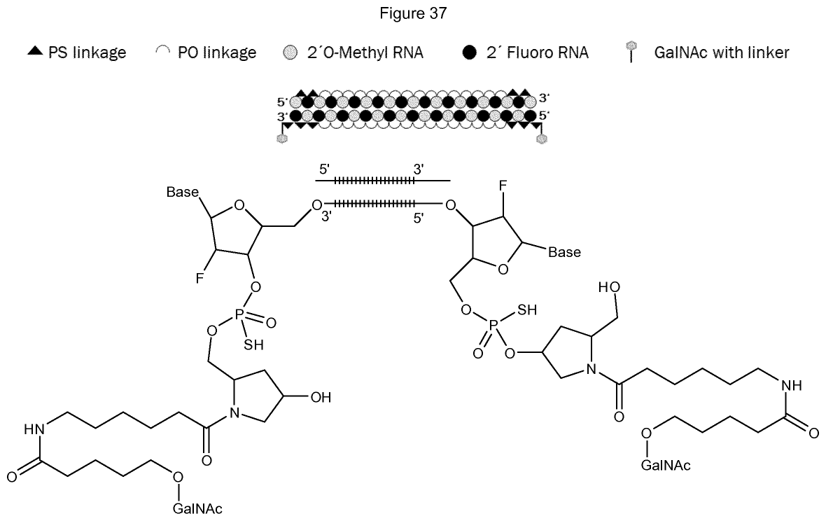

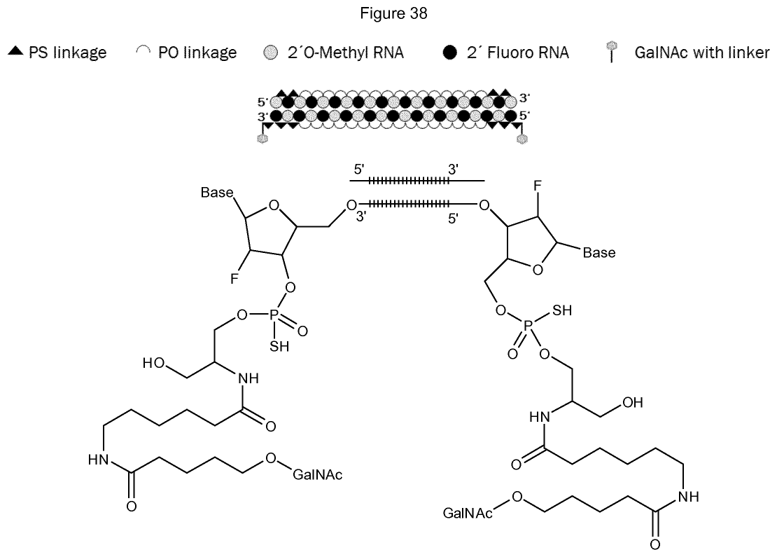

FIG. 31 shows the results from the stability studies in respect of Conjugates 2, 4, 5, 6 and 7. FIG. 32 shows the tritosome stability of Conjugates 2, 8, 9, 10, 11 and 12. FIG. 34 shows the tritosome stability of Conjugates 20, 21, 22, and 2.

All tested conjugates contain each one GalNAc linker unit at the 5′ end and another at the 3′ end of the second strand. The siRNAs are modified with alternating 2′-OMe/2′-F and contain each two phosphorothioate (PS) internucleotide linkages at their 5′ and 3′ terminal two internucleotide linkages, unless stated differently.

In Conjugate 4 the serinol-GalNAc units are attached via a phosphodiester bond. In Conjugate 5 the serinol-GalNAc units are conjugated via PS, whereas all internucleotide linkage in the second strand are phosphodiesters. In Conjugate 6 the second strand contains no PS. In Conjugate 7 two serinol-GalNAc units are attached to each second strand terminus and to each other via a PS-bonds at the respective ends. In Conjugate 8 a C6-amino-modifier at 5′ and a C7-amino-modifier at the 3′ end of the second strand were applied for ligand attachment. In Conjugate 9 Gly-C3-amino-modifiers, in Conjugate 10 piperidyl-amino-modifiers, in Conjugate 11 C3-amino-modifiers and in Conjugate 2 serinol-GalNAc units were used as linkers for conjugation to both ends of the second strand. In Conjugate 2 both terminal internucleotide as well as the nucleotide-serinol bonds are PS. In Conjugate 12 a C6-amino-modifier at the 5′ and a GlyC3-amino-modifier at the 3′ end of second strand were applied for ligand attachment. In Conjugate 20 Aspartol-amino modifiers were used to conjugate the GalNAc at the 5′ and 3′ of the sense strand. In Conjugate 21 serinol-C6-amino modifiers were used to conjugate the GalNAc at the 5′ and 3′ of the sense strand. In Conjugate 22 Hydroxyprolinol-amino modifiers were used to conjugate the GalNac at the 5′ and 3′ of the sense strand. The “ut” indicates an untreated sample which the other samples were normalised to.

The data show that in context of a serinol-derived linker moiety being provided at 5′ and 3′ ends of the sense strand, the number of phosphorothioate (PS) bonds between the terminal nucleotide and the linker, and/or between the terminal three nucleotides in the sense strand, can be varied, but have to be at least one in the 3′ and 5′-region of the sense strand, to maintain stability in tritosome lysates (FIG. 31). Moreover, the data show that it is not the nature of the linker that conveys improved stability, but the positioning at the 3′- and 5′-end of the sense strand (FIG. 32 and FIG. 34).

### Example 5—GalNAc siRNA Conjuqates with Specific Sense Strand Modification Patterns

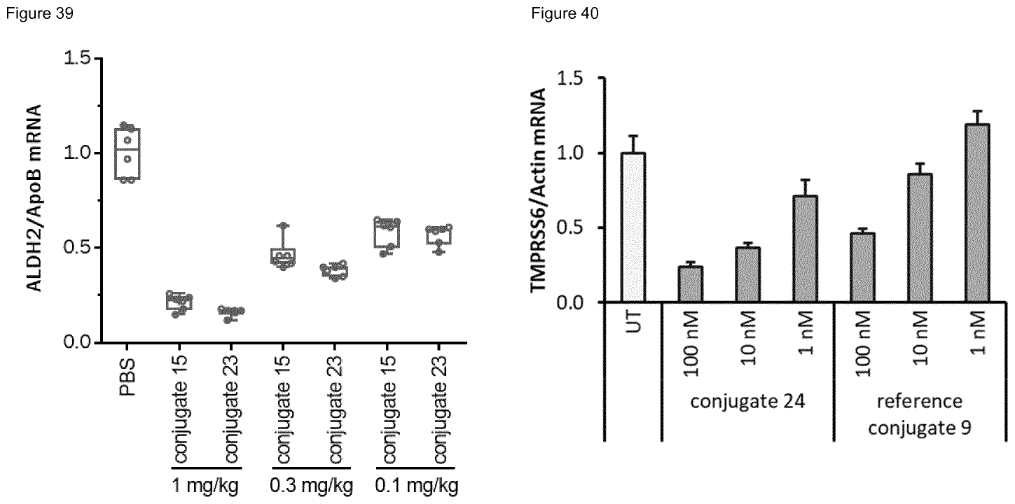

GalNAc conjugates with each one serinol-linked GalNAc moiety at both termini of the second strand and with 2′-F at positions 7-9 of the second strand reduce target mRNA levels in vitro and in vivo (FIG. 39, FIG. 40)

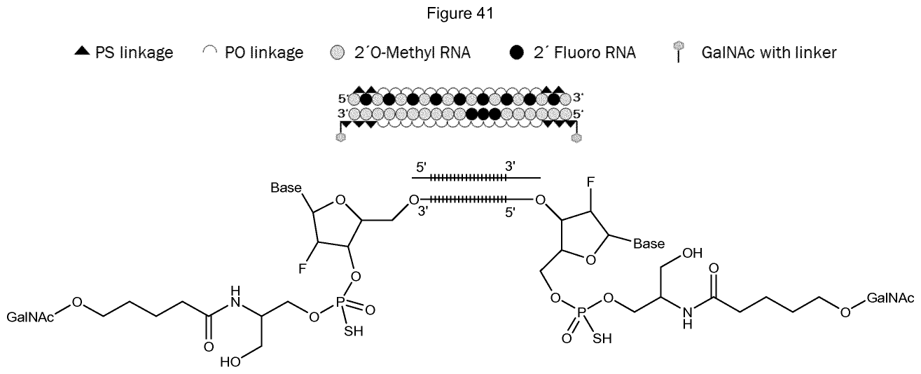

Different siRNA conjugates targeting human ALDH2 were tested for in vivo activity in mouse (FIG. 39). Both Conjugates 15 and 23 contain a similar first strand, which is modified with alternating 2′-OMe/2′-F. In both Conjugates 15 and 23, serinol-linked GalNAc moieties are conjugated to both ends of the second strand. In Conjugate 15, the second strand is modified with alternating 2′-F/2′-OMe. In Conjugate 23, the second strand is modified with 2′-OMe at positions 1-6 and 10-19 and with 2′-F at positions 7-9 (FIG. 41). Conjugate 23 mediates a stronger reduction of ALDH2 target gene levels as compared to Conjugate 15, indicating that a combination of the modification pattern and conjugation of Conjugate 23 result in a more portent siRNA. Here, C57BL/6 male mice were subcutaneously treated with 0.1, 1 and 3 mg/kg GalNAc conjugate. Liver sections were prepared 15 days after treatment, total RNA was extracted from the tissue and ALDH2 and ApoB mRNA levels were analyzed by Taqman qRT-PCR. Each bar represents mean±SD of six animals.

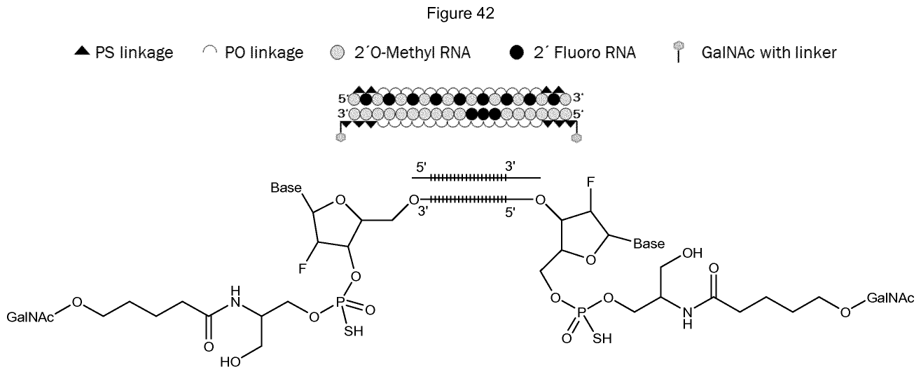

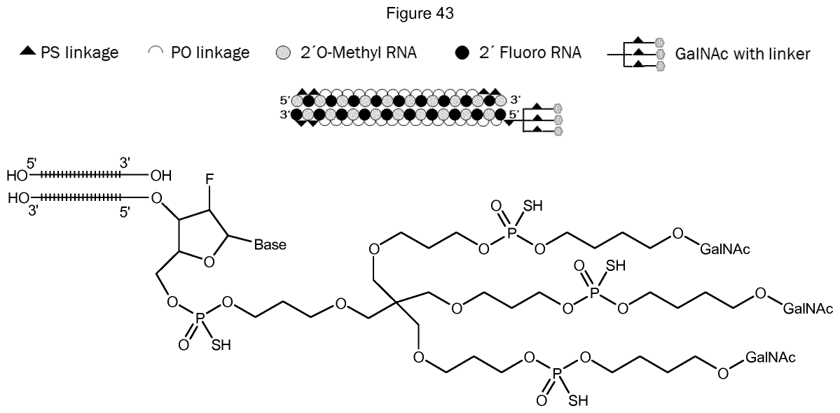

Different siRNA conjugates targeting human TMPRSS6 were tested for in vitro activity in mouse primary hepatocytes (FIG. 40). Reference conjugate 9 and Conjugate 24 contain a similar first strand, which is modified with alternating 2′-OMe/2′-F. In Reference conjugate 9, a triantennary GalNAc moiety is conjugated to the 5′ end of the second strand and the second strand is modified with alternating 2′-F/2′-OMe. In Conjugate 24, serinol-linked GalNAc moieties are conjugated to both ends of the second strand and the second strand is modified with 2′-OMe at positions 1-6 and 10-19 and with 2′-F at positions 7-9 (FIG. 42). All conjugates mediate dose-dependent reduction of TMPRSS6 target gene levels. Conjugate 24 has a better in vitro activity than Reference conjugate 9 (FIG. 40). “UT” indicates an untreated sample all other samples were normalized to. The experiment was conducted in mouse primary hepatocytes. The cells were seeded at a density of 20,000 cells per 96-well and treated with 1 nM, 10 nM, and 100 nM GalNAc conjugates directly after plating. Cells were lysed after 24 h, total RNA was extracted and TMPRSS6 and PTEN mRNA levels were determined by Taqman qRT-PCR. Each bar represents mean±SD from three technical replicates.

### Example 6

GalNac conjugates with each one serinol-linked GalNAc moiety at both termini of the second strand and with 2′-F at positions 7-9 of the second strand reduce TTR mRNA levels in vitro.

Different siRNA conjugates targeting human TTR were tested for in vitro activity in mouse primary hepatocytes. In all conjugates, serinol-linked GalNAc moieties are conjugated to both ends of the second strand. All conjugates X0383, X0385 and X0386 contain a similar first strand, which is modified with alternating 2′-OMe/2′-F. The second strand of X0383 is modified with alternating 2′-F/2′-OMe. The second strand of X0385 is modified with 2′-OMe at positions 1-6 and 10-19 and with 2′-F at positions 7-9. The second strand of X0386 is modified with 2′-OMe at positions 1-6 and 10-18 and with 2′-F at positions 7-9 and contains 3′-3′-linked RNA (“inverted RNA”) at position 19. All conjugates mediate dose-dependent reduction of TTR target gene levels. “UT” indicates an untreated sample all other samples were normalized to. “Luc” indicates a GalNAc-conjugated siRNA (X0028) targeting Luciferase, which was used as non-targeting control and does not reduce target mRNA levels.

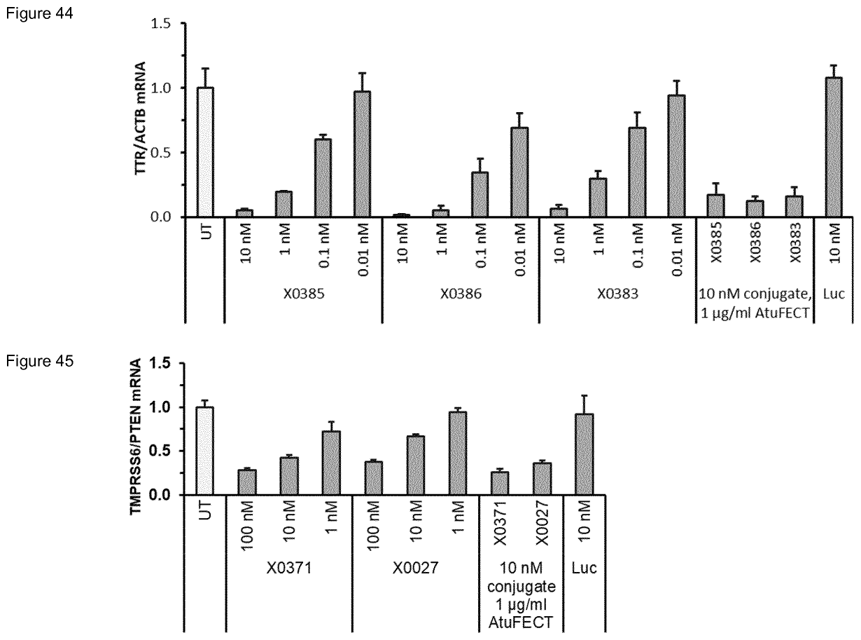

The experiment was conducted mouse primary Hepatocytes. The cells were seeded at a density of 20,000 cells per 96-well and treated with 10 nM, 1 nM, 0.1 nM and 0.01 nM GalNAc conjugated siRNAs directly after seeding. Transfections with 10 nM GalNAc-siRNA and 1 μg/ml Atufect liposomal transfection reagent served as control. Cells were lysed after 24 h, total RNA was extracted and TTR and ACTB mRNA levels were determined by Taqman qRT-PCR. Each bar represents mean±SD from three technical replicates. Data are shown in FIG. 44.

### Example 7

GalNac conjugates with each one serinol-linked GalNAc moiety at both termini of the second strand and with 2′-F at positions 7-9 of the second strand reduce TMPRSS6 mRNA levels in vitro.

Different siRNA conjugates targeting human TMPRSS6 were tested for in vitro activity in mouse primary hepatocytes. Both conjugates X0027 and X0371 contain a similar first strand, which is modified with alternating 2′-OMe/2′-F. In X0027, a triantennary GalNAc moiety is conjugated to the 5′ end of the second strand and the second strand is modified with alternating 2′-F/2′-OMe. In X0371, serinol-linked GalNAc moieties are conjugated to both ends of the second strand and the second strand is modified with 2′-OMe at positions 1-6 and 10-19 and with 2′-F at positions 7-9. All conjugates mediate dose-dependent reduction of TMPRSS6 target gene levels. X0371 has a slightly better in vitro activity than X0027. “UT” indicates an untreated sample all other samples were normalized to. “Luc” indicates a GalNAc-conjugated siRNA (X0028) targeting Luciferase, which was used as non-targeting control and does not reduce target mRNA levels.

The experiment was conducted in mouse primary hepatocytes. The cells were seeded at a density of 20,000 cells per 96-well and treated with 1 nM, 10 nM, and 100 nM GalNAc conjugates directly after plating. Transfections with 10 nM GalNAc-siRNA and 1 μg/ml Atufect liposomal transfection reagent served as control. Cells were lysed after 24 h, total RNA was extracted and TMPRSS6 and PTEN mRNA levels were determined by Taqman qRT-PCR. Each bar represents mean±SD from three technical replicates. Data are shown in FIG. 45.

### Example 8

GalNac conjugates with each one serinol-linked GalNAc moiety at both termini of the second strand and with 2′-F at positions 7-9 of the second strand reduce ALDH2 mRNA levels in vitro.

Different siRNA conjugates targeting human ALDH2 were tested for in vitro activity in mouse primary hepatocytes. Both conjugates X0320 and X0477 contain a similar first strand, which is modified with alternating 2′-OMe/2′-F. In both conjugates X0320 and X0477, serinol-linked GalNAc moieties are conjugated to both ends of the second strand. In X0320, the second strand is modified with alternating 2′-F/2′-OMe. In X0477, the second strand is modified with 2′-OMe at positions 1-6 and 10-19 and with 2′-F at positions 7-9. Both conjugates mediate dose-dependent reduction of ALDH2 target gene levels. “UT” indicates an untreated sample all other samples were normalized to. “Luc” indicates a GalNAc-conjugated siRNA (X0028) targeting Luciferase, which was used as non-targeting control and does not reduce target mRNA levels.

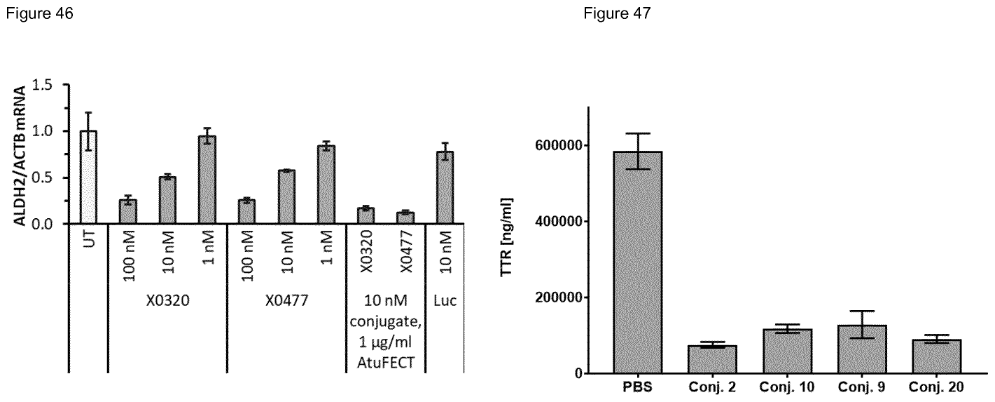

The experiment was conducted in mouse primary hepatocytes. The cells were seeded at a density of 20,000 cells per 96-well and treated with 1 nM, 10 nM, and 100 nM GalNAc conjugates directly after plating. Transfections with 10 nM GalNAc-siRNA and 1 μg/ml Atufect liposomal transfection reagent served as control. Cells were lysed after 24 h, total RNA was extracted and ALDH2 and ACTB mRNA levels were determined by Taqman qRT-PCR. Each bar represents mean±SD from three technical replicates. Data are shown in FIG. 46.

Sequence Table of conjugates of examples 6, 7 and 8

| | Single | |
|---|---|---|
| Duplex | strands | Sequence (A, first strand; B, second strand, both 5′-3′) |
| X0028 | X0028A | mU (ps) fC (ps) mG fA mA fG mU fA mU fU mC fC mG fC mG fU |
| | | mA (ps) fC (ps) mG |
| | X0028B | [ST23(ps)]3 ST41(ps) fC mG fU mA fC mG fC mG fG mA fA mU |
| | | fA mC fU mU fC (ps) mG (ps) fA |
| X0383 | X0383A | mU (ps) fU (ps) mA fU mA fG mA fG mC fA mA fG mA fA mC fA |
| | | mC (ps) fU (ps) mG |
| | X0383B | Ser(GN) (ps) fC (ps) mA (ps) fG mU fG mU fU mC fU mU fG mC |
| | | fU mC fU mA fU (ps) mA (ps) fA (ps) Ser(GN) |
| X0385 | X0385A | mU (ps) fU (ps) mA fU mA fG mA fG mC fA mA fG mA fA mC fA |
| | | mC (ps) fU (ps) mG |
| | X0385B | Ser(GN) (ps) mC ps) mA (ps) mG mU mG mU fU fC fU mU mG |
| | | mC mU mC mU mA mU (ps) mA (ps) mA (ps) Ser(GN) |
| X0386 | X0386A | mU (ps) fU (ps) mA fU mA fG mA fG mC fA mA fG mA fA mC fA |
| | | mC (ps) fU (ps) mG |
| | X0386B | Ser(GN) (ps) mC (ps) mA (ps) mG mU mG mU fU fC fU mU mG |
| | | mC mU mC mU mA mU mA irA (ps) Ser(GN) |
| X0027 | X0027A | mA (ps) fA (ps) mC fC mA fG mA fA mG fA mA fG mC fA mG fG |
| | | mU (ps) fG (ps) mA |
| | X0027B | [ST23 (ps)]3 ST41 (ps) fU (ps) mC (ps) fA mC fC mU fG mC fU |
| | | mU fC mU fU mC fU mG fG (ps) mU (ps) fU |
| X0371 | X0371A | mA (ps) fA (ps) mC fC mA fG mA fA mG fA mA fG mC fA mG fG |
| | | mU (ps) fG (ps) mA |
| | X0371B | Ser(GN) (ps) mU (ps) mC (ps) mA mC mC mU fG fC fU mU mC |
| | | mU mU mC mU mG mG (ps) mU (ps) mU (ps) Ser(GN) |
| X0320 | X0320A | mU (ps) fC (ps) mU fU mC fU mU fA mA fA mC fU mG fA mG fU |
| | | mU (ps) fU (ps) mC |
| | X0320B | Ser(GN) (ps) fG (ps) mA (ps) fA mA fC mU fC mA fG mU fU mU |
| | | fA mA fG mA fA (ps) mG (ps) fA (ps) Ser(GN) |
| X0477 | X0477A | mU (ps) fC (ps) mU fU mC fU mU fA mA fA mC fU mG fA mG fU |
| | | mU (ps) fU (ps) mC |
| | X0477B | Ser(GN) (ps) mG (ps) mA (ps) mA mA mC mU fC fA fG mU mU |
| | | mU mA mA mG mA mA (ps) mG (ps) mA (ps) Ser(GN) |

mA, mU, mC, mG 2′-OMe RNA
fA, fU, fC, fG 2′-deoxy-2′-F RNA
irA, irC, irU, irG inverted RNA (3′-3′ or 5′-5′)
(ps) phosphorothioate

Other abbreviations can be found in example 1

### Example 9

The comparable knock down level of serum TTR seven days post s.c. application of 0.3 mg/kg of conjugates 2, 9, 10 and 20 demonstrated in FIG. 47 indicates that the improved in vivo activity of conjugate 2 is not limited to the linker chemistry applied but essentially to the positioning at the 3′- and 5′-end of the sense strand.

All patents and patent applications referred to herein are incorporated by reference in their entirety. Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

### REFERENCES

1. Fire, A. et al. Nature 1998, 391 (6669), 806-11.
2. Elbashir, S. M. et al. Genes & development 2001, 15 (2), 188-200.
3. Matsuda et al. ACS Chem. Biol. 2015, 10, 1181-1187
4. Schmidt et al. Nucl. Acids Res. 2017, 45(5) 2294-2306
5. Kamiya et al. ChemBioChem 2014, 15, 2549-2555
6. Nair, J. K. et al. Journal of the American Chemical Society 2014, 136 (49), 6958-16961
7. Hövelmann, F. et al. Chemical Science 2016, 7 (1), 128-135.

TABLE
Summary sequence

| SEQ | | | |
|---|---|---|---|
| ID | | | Unmodified sequence 5′-3′ |
| NO: | Strand name | Sequence 5′-3′ | counterpart |
| 1 | X0371A | mA (ps) fA (ps) mC fC mA fG mA fA mG fA mA fG mC | AACCAGAAGAAGCAGGUGA |
| | | fA mG fG mU (ps) fG (ps) mA | |
| 2 | X0371B | Ser(GN) (ps) mU (ps) mC (ps) mA mC mC mU fG fC fU | UCACCUGCUUCUUCUGGUU |
| | | mU mC mU mU mC mU mG mG (ps) mU (ps) mU (ps) Ser(GN) | |
| 3 | TTR fw | TGGACACCAAATCGTACTGGAA | TGGACACCAAATCGTACTGGAA |
| 4 | TTR rev | TGGACACCAAATCGTACTGGAA | TGGACACCAAATCGTACTGGAA |
| 5 | TTR probe | BHQ1-ACTTGGCATTTCCCCGTTCCATGAATT-FAM | ACTTGGCATTTCCCCGTTCCATG |
| | | | AATT |
| 6 | PTEN fw | CACCGCCAAATTTAACTGCAGA | CACCGCCAAATTTAACTGCAGA |
| 7 | PTEN rev | AAGGGTTTGATAAGTTCTAGCTGT | AAGGGTTTGATAAGTTCTAGCTGT |
| 8 | PTEN probe | BHQ1-TGCACAGTATCCTTTTGAAGACCATAACCCA-YY | TGCACAGTATCCTTTTGAAGACC |
| | | | ATAACCCA |
| 9 | ALDH2 fw | GGCAAGCCTTATGTCATCTCGT | GGCAAGCCTTATGTCATCTCGT |
| 10 | ALDH2 rev | GGAATGGTTTTCCCATGGTACTT | GGAATGGTTTTCCCATGGTACTT |
| 11 | ALDH2 probe | BHQ1-TGAAATGTCTCCGCTATTACGCTGGCTG-FAM | TGAAATGTCTCCGCTATTACGCT |
| | | | GGCTG |
| 12 | TMPRSS6 fw | CGGCACCTACCTTCCACTCTT | CGGCACCTACCTTCCACTCTT |
| 13 | TMPRSS6 rev | TCGGTGGTGGGCATCCT | TCGGTGGTGGGCATCCT |
| 14 | TMPRSS 6 | BHQ1-CCGAGATGTTTCCAGCTCCCCTGTTCTA-FAM | CCGAGATGTTTCCAGCTCCCCTG |
| | probe | | TTCTA |
| 15 | LPA fw | GTGTCCTCGCAACGTCCA | GTGTCCTCGCAACGTCCA |
| 16 | LPA rev | GACCCCGGGGCTTTG | GACCCCGGGGCTTTG |
| 17 | LPA probe | BHQ1-TGGCTGTTTCTGAACAAGCACCAATGG-FAM | TGGCTGTTTCTGAACAAGCACCA |
| | | | ATGG |
| 18 | ACTB fw | GCATGGGTCAGAAGGATTCCTAT | GCATGGGTCAGAAGGATTCCTAT |
| 19 | ACTB rev | TGTAGAAGGTGTGGTGCCAGATT | TGTAGAAGGTGTGGTGCCAGATT |
| 20 | ACTB probe | BHQ1-TCGAGCACGGCATCGTCACCAA-YY | TCGAGCACGGCATCGTCACCAA |
| 21 | Conjugate 1 AS | mU (ps) fU (ps) mA fU mA fG mA fG mC fA mA fG mA | UUAUAGAGCAAGAACACUGUU |
| | | fA mC fA mC fU mG (ps) fU (ps) mU (ps) Ser(GN) | |
| 22 | Conjugate 1 S | Ser(GN) (ps) fA mA fC mA fG mU fG mU fU mC fU mU | AACAGUGUUCUUGCUCUAUAA |
| | | fG mC fU mC fU mA fU (ps) mA (Ps) fA | |
| 23 | Conjugate 2 AS | mU (ps) fU (ps) mA fU mA fG mA fG mC fA mA fG mA | UUAUAGAGCAAGAACACUGUU |
| | | fA mC fA mC fU mG (ps) fu (ps) mu | |
| 24 | Conjugate 2 S | Ser(GN) (ps) fA (ps) mA (ps) fC mA fG mU fG mU fU | AACAGUGUUCUUGCUCUAUAA |
| | | mC fU mU fG mC fU mC fU mA fU (ps) mA (ps) fA (ps) | |
| | | Ser(GN) | |
| 25 | Conjugate 3 AS | mU (ps) fU (ps) mA fU mA fG mA fG mC fA mA fG mA | UUAUAGAGCAAGAACACUGUU |
| | | fA mC fA mC fU mG (ps) fU (ps) mU (ps) Ser(GN) | |
| 26 | Conjugate 3 S | Ser(GN) (ps) fA (ps) mA (ps) fC mA fG mU fG mU fU | AACAGUGUUCUUGCUCUAUAA |
| | | mC fU mU fG mC fU mC fU mA fU (ps) mA (ps) fA (ps) | |
| | | Ser(GN) | |
| 27 | Conjugate 4 AS | mU (ps) fU (ps) mA fU mA fG mA fG mC fA mA fG mA | UUAUAGAGCAAGAACACUGUU |
| | | fA mC fA mC fU mG (ps) fu (ps) mu | |
| 28 | Conjugate 4 S | Ser(GN) fA (ps) mA (ps) fC mA fG mU fG mU fU mC | AACAGUGUUCUUGCUCUAUAA |
| | | fU mU fG mC fU mC fU mA fU (ps) mA (ps) fA Ser(GN) | |
| 29 | Conjugate 5 AS | mU (ps) fU (ps) mA fU mA fG mA fG mC fA mA fG mA | UUAUAGAGCAAGAACACUGUU |
| | | fA mC fA mC fU mG (ps) fu (ps) mu | |
| 30 | Conjugate 5 S | Ser(GN) (ps) fA mA fC mA fG mU fG mU fU mC fU mU | AACAGUGUUCUUGCUCUAUAA |
| | | fG mC fU mC fU mA fU mA fA (ps) Ser(GN) | |
| 31 | Conjugate 6 AS | mU (ps) fU (ps) mA fU mA fG mA fG mC fA mA fG mA | UUAUAGAGCAAGAACACUGUU |
| | | fA mC fA mC fU mG (ps) fu (ps) mu | |
| 32 | Conjugate 6 S | Ser(GN) fA mA fC mA fG mU fG mU fU mC fU mU fG mC | AACAGUGUUCUUGCUCUAUAA |
| | | fU mC fU mA fU mA fA Ser(GN) | |
| 33 | Conjugate 7 AS | mU (ps) fU (ps) mA fU mA fG mA fG mC fA mA fG mA | UUAUAGAGCAAGAACACUGUU |
| | | fA mC fA mC fU mG (ps) fu (ps) mu | |
| 34 | Conjugate 7 S | Ser(GN) (ps) Ser(GN) (ps) fA (ps) mA (ps) fC mA | AACAGUGUUCUUGCUCUAUAA |
| | | fG mU fG mU fU mC fU mU fG mC fU mC fU mA fU (ps) | |
| | | mA (ps) fA (ps) Ser(GN) (ps) Ser(GN) | |
| 35 | Conjugate 8 AS | mU (ps) fU (ps) mA fU mA fG mA fG mC fA mA fG mA | UUAUAGAGCAAGAACACUGUU |
| | | fA mC fA mC fU mG (ps) fu (ps) mu | |
| 36 | Conjugate 8 S | C6Am(GN) (ps) fA (ps) mA (ps) fC mA fG mU fG mU | AACAGUGUUCUUGCUCUAUAA |
| | | fU mC fU mU fG mC fU mC fU mA fU (ps) mA (ps) fA | |
| | | (ps) C7Am(GN) | |
| 37 | Conjugate 9 AS | mU (ps) fU (ps) mA fU mA fG mA fG mC fA mA fG mA | UUAUAGAGCAAGAACACUGUU |
| | | fA mC fA mC fU mG (ps) fu (ps) mu | |
| 38 | Conjugate 9 S | GlyC3Am(GN) (ps) fA (ps) mA (ps) fC mA fG mU fG | AACAGUGUUCUUGCUCUAUAA |
| | | mU fU mC fU mU fG mC fU mC fU mA fU (ps) mA (ps) | |
| | | fA (ps) GlyC3Am(GN) | |
| 39 | Conjugate 10 | mU (ps) fU (ps) mA fU mA fG mA fG mC fA mA fG mA | UUAUAGAGCAAGAACACUGUU |
| | AS | fA mC fA mC fU mG (ps) fU (ps) mU | |
| 40 | Conjugate 10 S | PipAm(GN) (ps) fA (ps) mA (ps) fC mA fG mU fG mU | AACAGUGUUCUUGCUCUAUAA |
| | | fU mC fU mU fG mC fU mC fU mA fU (ps) mA (ps) fA | |
| | | (ps) PipAm(GN) | |
| 41 | Conjugate 11 | mU (ps) fU (ps) mA fU mA fG mA fG mC fA mA fG mA | UUAUAGAGCAAGAACACUGUU |
| | AS | fA mC fA mC fU mG (ps) fU (ps) mU | |
| 42 | Conjugate 11 S | C3Am(GN) (ps) fA (ps) mA (ps) fC mA fG mU fG mU | AACAGUGUUCUUGCUCUAUAA |
| | | fU mC fU mU fG mC fU mC fU mA fU (ps) mA (ps) fA | |
| | | (ps) C3Am(GN) | |
| 43 | Conjugate 12 | mU (ps) fU (ps) mA fU mA fG mA fG mC fA mA fG mA | UUAUAGAGCAAGAACACUGUU |
| | AS | fA mC fA mC fU mG (ps) fU (ps) mU | |
| 44 | Conjugate 12 S | C6Am(GN) (ps) fA (ps) mA (ps) fC mA fG mU fG mU | AACAGUGUUCUUGCUCUAUAA |
| | | fU mC fU mU fG mC fU mC fU mA fU (ps) mA (ps) fA | |
| | | (ps) GlyC3Am(GN) | |
| 45 | Conjugate 15 | mU (ps) fC (ps) mU fU mC fU mU fA mA fA mC fU mG | UCUUCUUAAACUGAGUUUC |
| | AS | fA mG fU mU (ps) fU (ps) mC | |
| 46 | Conjugate 15 S | Ser(GN) (ps) fG (ps) mA (ps) fA mA fC mU fC mA | GAAACUCAGUUUAAGAAGA |
| | | fG mU fU mU fA mA fG mA fA (ps) mG (ps) fA (ps) | |
| | | Ser(GN) | |
| 47 | Conjugate 16 | mA (ps) fU (ps) mG fU mA fG mC fC mG fA mG fG mA | AUGUAGCCGAGGAUCUUCU |
| | AS | fU mC fU mU (ps) fC AS (ps) mU | |
| 48 | Conjugate 16 S | Ser(GN) (ps) fA (ps) mG (ps) fA mA fG mA fU mC fC | AGAAGAUCCUCGGCUACAU |
| | | mU fC mG fG mC fU mA fC (ps) mA (ps) fU (ps) Ser | |
| | | (GN) | |
| 49 | Conjugate 18 | mA (ps) fA (ps) mC fC mA fG mA fA mG fA mA fG mC | AACCAGAAGAAGCAGGUGA |
| | AS | fA mG fG mU (ps) fG (ps) AS mA | |
| 50 | Conjugate 18 S | Ser(GN) (ps) fU (ps) mC (ps) fA mC fC mU fG mC fU | UCACCUGCUUCUUCUGGUU |
| | | mU fC mU fU mC fU mG fG (ps) mU (ps) fU (ps) Ser | |
| | | (GN) | |
| 51 | Conjugate 19 | mA (ps) fU (ps) mA fA mC fU mC fU mG fU mC fC mA | AUAACUCUGUCCAUUACCG |
| | AS | fU mU fA mC (ps) fC (ps) mG | |
| 52 | Conjugate 19 S | Ser(GN) (ps) fC (ps) mG (ps) fG mU fA mA fU mG fG | CGGUAAUGGACAGAGUUAU |
| | | mA fC mA fG mA fG mU fU (ps) mA (ps) fU (ps) Ser | |
| | | (GN) | |
| 53 | Conjugate 20 | mU (ps) fU (ps) mA fU mA fG mA fG mC fA mA fG mA | UUAUAGAGCAAGAACACUGUU |
| | AS | fA mC fA mC fU mG (ps) fU (ps) mU | |
| 54 | Conjugate 20 S | Asp(GN) (ps) fA (ps) mA (ps) fC mA fG mU fG mU fU | AACAGUGUUCUUGCUCUAUAA |
| | | mC fU mU fG mC fU mC fU mA fU (ps) mA (ps) fA (ps) | |
| | | Asp(GN) | |
| 55 | Conjugate 21 | mU (ps) fU (ps) mA fU mA fG mA fG mC fA mA fG mA | UUAUAGAGCAAGAACACUGUU |
| | AS | fA mC fA mC fU mG (ps) fU (ps) mU | |
| 56 | Conjugate 21 S | Hpro(GN) (ps) fA (ps) mA (ps) fC mA fG mU fG mU | AACAGUGUUCUUGCUCUAUAA |
| | | fU mC fU mU fG mC fU mC fU mA fU (ps) mA (ps) fA | |
| | | (ps) Hpro(GN) | |
| 57 | Conjugate 22 | mU (ps) fU (ps) mA fU mA fG mA fG mC fA mA fG mA | UUAUAGAGCAAGAACACUGUU |
| | AS | fA mC fA mC fU mG (ps) AS fU (ps) mU | |
| 58 | Conjugate 22 S | SerC6(GN) (ps) fA (ps) mA (ps) fC mA fG mU fG mU | AACAGUGUUCUUGCUCUAUAA |
| | | fU mC fU mU fG mC fU mC fU mA fU (ps) mA (ps) fA | |
| | | (ps) SerC6(GN) | |
| 59 | Conjugate 23 | mU (ps) fC (ps) mU fU mC fU mU fA mA fA mC fU mG | UCUUCUUAAACUGAGUUUC |
| | AS | fA mG fU mU (ps) fU (ps) mC | |
| 60 | Conjugate 23 S | Ser(GN) (ps) mG (ps) mA (ps) mA mA mC mU fC fA fG | GAAACUCAGUUUAAGAAGA |
| | | mU mU mU mA mA mG mA mA (ps) mG (ps) mA (ps) Ser | |
| | | (GN) | |
| 61 | Conjugate 24 | mA (ps) fA (ps) mC fC mA fG mA fA mG fA mA fG mC | AACCAGAAGAAGCAGGUGA |
| | AS | fA mG fG mU (ps) fG (ps) mA | |
| 62 | Conjugate 24 S | Ser(GN) (ps) mU (ps) mC (ps) mA mC mC mU fG fC fU | UCACCUGCUUCUUCUGGUU |
| | | mU mC mU mU mC mU mG mG (ps) mU (ps) mU (ps) Ser | |
| | | (GN) | |
| 63 | Reference | mU (ps) fU (ps) mA fU mA fG mA fG mC fA mA fG mA | UUAUAGAGCAAGAACACUGUU |
| | Conjugate 1 AS | fA mC fA mC fU mG (ps) fU (ps) mU | |
| 64 | Reference | Ser(GN) (ps) fA mA fC mA fG mU fG mU fU mC fU mU | AACAGUGUUCUUGCUCUAUAA |
| | Conjugate 1 S | fG mC fU mC fU mA fU (ps) mA (ps) fA | |
| 65 | Reference | mU (ps) fU (ps) mA fU mA fG mA fG mC fA mA fG mA | UUAUAGAGCAAGAACACUGUU |
| | Conjugate 2 AS | fA mC fA mC fU mG (ps) fU (ps) mU (ps) Ser(GN) | |
| 66 | Reference | fA (ps) mA (ps) fC mA fG mU fG mU fU mC fU mU fG | AACAGUGUUCUUGCUCUAUAA |
| | Conjugate 2 S | mC fU mC fU mA fU (ps) mA (ps) fA | |
| 67 | Reference | mU (ps) fC (ps) mG fA mA fG mU fA mU fU mC fC mG | UCGAAGUAUUCCGCGUACG |
| | Conjugate 3 AS | fC mG fU mA (ps) fC (ps) mG | |
| 68 | Reference | [(ST23) (ps)]₃ C4XLT (ps) fC mG fU mA fC mG fC mG | CGUACGCGGAAUACUUCGA |
| | Conjugate 3 S | fG mA fA mU fA mC fU mU fC (ps) mG (ps) fA | |
| 69 | Reference | mU (ps) fU (ps) mA fU mA fG mA fG mC fA mA fG mA | UUAUAGAGCAAGAACACUGUU |
| | Conjugate 4 AS | fA mC fA mC fU mG (ps) fU (ps) mU | |
| 70 | Reference | [(ST23) (ps)]₃ C4XLT(ps)fA (ps) mA (ps) fC mA fG | AACAGUGUUCUUGCUCUAUAA |
| | Conjugate 4 S | mU fG mU fU mC fU mU fG mC fU mC fU mA fU (ps) mA | |
| | | (ps) fA | |
| 71 | Reference | mC (ps) fU (ps) mU fA mC fU mC fU mC fG mC fC mC | CUUACUCUCGCCCAAGCGA |
| | Conjugate 5 AS | fA mA fG mC (ps) fG (ps) mA | |
| 72 | Reference | [(ST23) (ps)]3 (C6XLT) (ps) fU mC fG mC fU mU fG | UCGCUUGGGCGAGAGUAAG |
| | Conjugate 5 S | mG fG mC fG mA fG mA fG mU fA (ps) mA (ps) fG | |
| 73 | Reference | mU (ps) fC (ps) mU fU mC fU mU fA mA fA mC fU mG | UCUUCUUAAACUGAGUUUC |
| | Conjugate 6 AS | fA mG fU mU (ps) fU (ps) mC | |
| 74 | Reference | [ST23 (ps)]3 ltrb (ps) fG mA fA mA fC mU fC mA | GAAACUCAGUUUAAGAAGA |
| | Conjugate 6 S | fG mU fU mU fA mA fG mA fA (ps) mG (ps) fA | |
| 75 | Reference | mA (ps) fU (ps) mG fU mA fG mC fC mG fA mG fG mA | AUGUAGCCGAGGAUCUUCU |
| | Conjugate 7 AS | fU mC fU mU (ps) fC (ps) mU | |
| 76 | Reference | [ST23 (ps)]3 ltrb (ps) fA mG fA mA fG mA fU mC | AGAAGAUCCUCGGCUACAU |
| | Conjugate 7 S | fC mU fC mG fG mC fU mA fC (ps) mA (ps) fU | |
| 77 | Reference | mU (ps) fA (ps) mC fC mA fG mA fA mG fA mA fG mC | UACCAGAAGAAGCAGGUGA |
| | Conjugate 8 AS | fA mG fG mU (ps) fG (ps) mA | |
| 78 | Reference | [ST23 (ps)]3 ST41 (ps)fU mC fA mC fC mU fG mC fU | UCACCUGCUUCUUCUGGUA |
| | Conjugate 8 S | mU fC mU fU mC fU mG fG (ps) mU (ps) fA | |
| 79 | Reference | mA (ps) fA (ps) mC fC mA fG mA fA mG fA mA fG mC | AACCAGAAGAAGCAGGUGA |
| | Conjugate 9 AS | fA mG fG mU (ps) fG (ps) mA | |
| 80 | Reference | [ST23 (ps)]3 ST41 (ps) fU mC fA mC fC mU fG mC | UCACCUGCUUCUUCUGGUU |
| | Conjugate 9 S | fU mU fC mU fU mC fU mG fG (ps) mU (ps) fU | |
| 81 | X0028A | mU (ps) fC (ps) mG fA mA fG mU fA mU fU mC fC mG | UCGAAGUAUUCCGCGUACG |
| | | fC mG fU mA (ps) fC (ps) mG | |
| 82 | X0028B | [ST23(ps)]3 ST41(ps) fC mG fU mA fC mG fC mG fG | CGUACGCGGAAUACUUCGA |
| | | mA fA mU fA mC fU mU fC (ps) mG (ps) fA | |
| 83 | X0383A | mU (ps) fU (ps) mA fU mA fG mA fG mC fA mA fG mA | UUAUAGAGCAAGAACACUG |
| | | fA mC fA mC (ps) fU (ps) mG | |
| 84 | X0383B | Ser(GN) (ps) fC (ps) mA (ps) fG mU fG mU fU mC | CAGUGUUCUUGCUCUAUAA |
| | | fU mU fG mC fU mC fU mA fU (ps) mA (ps) fA (ps) | |
| | | Ser(GN) | |
| 85 | X0385A | mU (ps) fU (ps) mA fU mA fG mA fG mC fA mA fG mA | UUAUAGAGCAAGAACACUG |
| | | fA mC fA mC (ps) fU (ps) mG | |
| 86 | X0385B | Ser(GN) (ps) mC ps) mA (ps) mG mU mG mU fU fC fU | CAGUGUUCUUGCUCUAUAA |
| | | mU mG mC mU mC mU mA mU (ps) mA (ps) mA (ps) Ser | |
| | | (GN) | |
| 87 | X0386A | mU (ps) fU (ps) mA fU mA fG mA fG mC fA mA fG mA | UUAUAGAGCAAGAACACUG |
| | | fA mC fA mC (ps) fU (ps) mG | |
| 88 | X0386B | Ser(GN) (ps) mC (ps) mA (ps) mG mU mG mU fU fC | CAGUGUUCUUGCUCUAUAA |
| | | fU mU mG mC mU mC mU mA mU mA irA (ps) Ser(GN) | |
| 89 | X0027A | mA (ps) fA (ps) mC fC mA fG mA fA mG fA mA fG mC | AACCAGAAGAAGCAGGUGA |
| | | fA mG fG mU (ps) fG (ps) mA | |
| 90 | X0027B | [ST23 (ps)]3 ST41 (ps) fU (ps) mC (ps) fA mC fC | UCACCUGCUUCUUCUGGUU |
| | | mU fG mC fU mU fC mU fU mC fU mG fG (ps) mU (ps) | |
| | | fU | |
| 91 | X0371A | mA (ps) fA (ps) mC fC mA fG mA fA mG fA mA fG mC | AACCAGAAGAAGCAGGUGA |
| | | fA mG fG mU (ps) fG (ps) mA | |
| 92 | X0371B | Ser(GN) (ps) mU (ps) mC (ps) mA mC mC mU fG fC | UCACCUGCUUCUUCUGGUU |
| | | fU mU mC mU mU mC mU mG mG (ps) mU (ps) mU (ps) | |
| | | Ser(GN) | |
| 93 | X0320A | mU (ps) fC (ps) mU fU mC fU mU fA mA fA mC fU mG | UCUUCUUAAACUGAGUUUC |
| | | fA mG fU mU (ps) fU (ps) mC | |
| 94 | X0320B | Ser(GN) (ps) fG (ps) mA (ps) fA mA fC mU fC mA | GAAACUCAGUUUAAGAAGA |
| | | fG mU fU mU fA mA fG mA fA (ps) mG (ps) fA (ps) | |
| | | Ser(GN) | |
| 95 | X0477A | mU (ps) fC (ps) mU fU mC fU mU fA mA fA mC fU mG | UCUUCUUAAACUGAGUUUC |
| | | fA mG fU mU (ps) fU (ps) mC | |
| 96 | X0477B | Ser(GN) (ps) mG (ps) mA (ps) mA mA mC mU fC fA | GAAACUCAGUUUAAGAAGA |
| | | fG mU mU mU mA mA mG mA mA (ps) mG (ps) mA (ps) | |
| | | Ser(GN) | |

1. A conjugate for inhibiting expression of a target gene in a cell, particularly in a hepatocyte, said conjugate comprising a nucleic acid portion and ligand portions, said nucleic acid portion comprising at least one duplex region that comprises at least a portion of a first RNA strand and at least a portion of a second RNA strand that is at least partially complementary to the first strand, wherein said first strand is at least partially complementary to at least a portion of RNA transcribed from said target gene, said ligand portions comprising a linker moiety and a targeting ligand for in vivo targeting of cells and being conjugated exclusively to the 3′ and/or 5′ ends of one or both RNA strands, wherein the 5′ end of the first RNA strand is not conjugated, wherein:
(i) the second RNA strand is conjugated at the 5′ end to the targeting ligand, and wherein (a) the second RNA strand is also conjugated at the 3′ end to the targeting ligand and the 3′ end of the first RNA strand is not conjugated; or (b) the first RNA strand is conjugated at the 3′ end to the targeting ligand and the 3′ end of the second RNA strand is not conjugated; or (c) both the second RNA strand and the first RNA strand are also conjugated at the 3′ ends to the targeting ligand; or
(ii) both the second RNA strand and the first RNA strand are conjugated at the 3′ ends to the targeting ligand and the 5′ end of the second RNA strand is not conjugated.

2. The conjugate of claim 1, wherein the second RNA strand is conjugated at the 5′ end to the targeting ligand, the second RNA strand is also conjugated at the 3′ end to the targeting ligand and the 3′ end of the first RNA strand is not conjugated.

3. The conjugate of any preceding claim, wherein the targeting ligands are GalNAc moieties.

4. The conjugate of any preceding claim, wherein the first RNA strand is a compound of formula (IX): 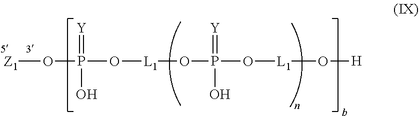
wherein b is 0 or 1; and
the second RNA strand is a compound of formula (X): 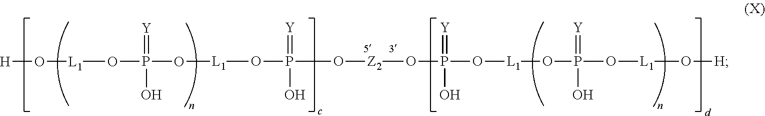
wherein:
c and d are independently 0 or 1;
Z₁ and Z₂ are the RNA portions of the first and second RNA strands respectively;
Y is O or S;
n is 0, 1, 2 or 3;
L1 is a linker to which a ligand is attached; and
wherein b+c+d is 2 or 3, preferably 2.

5. The conjugate of claims 1, 3 and 4, wherein the first strand is a compound of formula (XII) 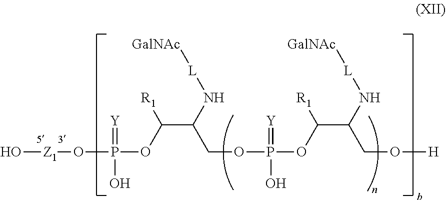
wherein b is preferably 0 or 1; and
the second strand is a compound of formula (XIII): 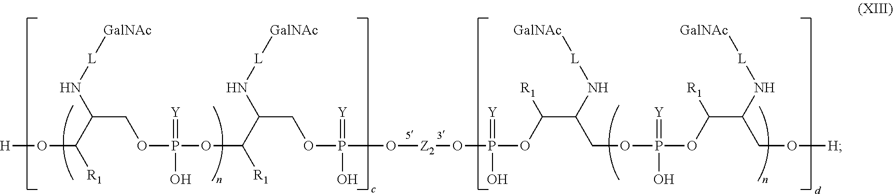
wherein c and d are independently preferably 0 or 1;
wherein:
Z₁ and Z₂ are respectively the first and second strand of the nucleic acid;
Y is independently O or S;
R₁ is H or methyl, preferably H;
n is independently preferably 0, 1, 2 or 3; and
L is the same or different in formulae (XII) and (XIII), and is the same or different within formulae (XII) and (XIII) when L is present more than once within the same formula, and is preferably selected from the group comprising, or preferably consisting of:
—(CH₂)ᵣC(O)—, wherein r=2-12;
(CH₂—CH₂—O)ₛ—CH₂—C(O)—, wherein s=1-5;
(CH₂)ₜ—CO—NH—(CH₂)ₜ—NH—C(O)—, wherein t is independently 1-5;
(CH₂)ᵤ—CO—NH—(CH₂)ᵤ—C(O)—, wherein u is independently 1-5; and
(CH₂)ᵥ—NH—C(O)—, wherein v is 2-12; and
wherein the terminal C(O), if present, is attached to the NH group of the linker;
and wherein b+c+d is preferably 2 or 3.

6. The conjugate of claims 4 and 5, wherein b is 0, c is 1, d is 1 and n is 0.

7. The conjugate of claims 4-6, wherein Y is S.

8. The conjugate of any preceding claim, wherein the targeted cells are hepatocytes.

9. The conjugate of any preceding claim, wherein the nucleic acid portion comprises between one to ten phosphorothioate internucleotide linkages.

10. The conjugate of any preceding claim, wherein the first strand of the nucleic acid includes modified nucleotides at a plurality of positions, and wherein the nucleotides at positions 2 and 14 from the 5′ end of the first strand are not modified with a 2′-OMe modification.

11. The conjugate of any preceding claim, wherein the nucleotides at positions 2 and 14 from the 5′ end of the first strand are not modified with a 2′-OMe modification, and the nucleotide on the second strand which corresponds to position 11, or 13, or 11 and 13, or 11-13 of the first strand is/are not modified with a 2′-OMe modification.

12. The conjugate of any preceding claim, wherein the nucleotides at positions 2 and 14 from the 5′ end of the first strand are modified with a 2′-F modification, and the nucleotides on the second strand which correspond to position 11, or 13, or 11 and 13, or 11-13 of the first strand are modified with a 2′-F modification.

13. The conjugate of any preceding claim, wherein the conjugate is an siRNA.

14. A pharmaceutical composition comprising a conjugate of any preceding claim together with a pharmaceutically acceptable diluent or carrier.

15. The conjugate of claims 1-13 or the pharmaceutical composition of claim 14 for use in medicine.
